(19) United States
(12) Patent Application Publication
(10) Pub. No.: US 2012/0135025 A1
(43) Pub. Date: May 31, 2012

(54) CHLAMYDIA ANTIGENS AND USES THEREOF

(75) Inventors: Jessica Baker Flechtner, Maynard, MA (US); Kenya Prince Cohane, Worcester, MA (US); Todd Gierahn, Brookline, MA (US); Alexander Yao-Hsien Lee, Cambridge, MA (US); George Rainer Siber, New York, NY (US)

(73) Assignee: GENOCEA BIOSCIENCES, INC., Cambridge, MA (US)

(21) Appl. No.: 13/277,996

(22) Filed: Oct. 20, 2011

Related U.S. Application Data
(60) Provisional application No. 61/405,162, filed on Oct. 20, 2010.

(51) Int. Cl. A61K 39/118; A61P 31/04; A61P 37/04; C07K 14/295; C12N 15/31

(52) U.S. Cl. 424/190.1; 530/350; 536/23.7

(57) ABSTRACT

The present invention provides novel chlamydia antigens, nucleic acids encoding the antigens, and immunogenic compositions including the antigens. The present invention further provides methods of using the antigens to elicit immune responses (e.g., T cell-mediated and/or B cell-mediated immune responses). The present invention provides methods of prophylaxis and/or treatment of chlamydia-mediated diseases comprising administering an immunogenic composition including one or more of the novel antigens described herein.

### CROSS REFERENCE TO RELATED APPLICATIONS

This application claims priority to U.S. Provisional Patent Application Ser. No. 61/405,162, filed Oct. 20, 2010, the entirety of which is hereby incorporated by reference.

### BACKGROUND

Chlamydia trachomatis is an obligate intracellular bacterium which exists as multiple serovariants with distinct tropism for the eye or urogenital tract. Infection with urogenital variants can cause various disease conditions such as urethritis, cervicitis, pharyngitis, proctitis, epididymitis, and prostatitis. Untreated chlamydial infection can cause pelvic inflammatory disease, which in turn can lead to ectopic pregnancy, infertility, and chronic pelvic pain. Infection during pregnancy has been linked to severe complications such as spontaneous abortion, premature delivery, premature rupture of fetal membranes, low birth weight, and neonatal infections (Navarro et al., Can. J. Inf. Dis. 13(3):195-207, 2002). Infection with ocular variants of C. trachomatis can cause trachoma, or conjunctivitis of eyelid and corneal surfaces, and is a leading cause of preventable blindness. Pathological effects of C. trachomatis in humans are a significant societal economic burden as well as an ongoing public health concern in both industrialized and developing nations. An estimated four to five million new cases of chlamydial infection occur each year in the United States alone. The annual costs of treating pelvic inflammatory disease may be as high as US $10 billion. The prevalence of C. trachomatis infection in the developing world is over 90%, with an estimated 500 million people at high risk for infection (World Health Organization, Sexually Transmitted Diseases, 2008). There is an urgent need for immunogenic, effective vaccines for controlling chlamydial infections worldwide.

### SUMMARY

The present invention encompasses the discovery of novel antigens from Chlamydia trachomatis that elicit antigen specific immune responses in mammals. Such novel antigens, and/or nucleic acids encoding the antigens, can be incorporated into immunogenic compositions and administered to elicit immune responses, e.g., to provide protection against chlamydia infections and disease caused by chlamydia organisms. Such novel antigens, and/or responses to novel antigens, can be detected to identify and/or characterize immune responses to chlamydia organisms.

Accordingly, in one aspect, the invention provides immunogenic compositions (e.g., vaccines) comprising an isolated chlamydia antigen selected from a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, a CT476 polypeptide antigen, and combinations thereof. In some embodiments, a chlamydia antigen comprises a full-length chlamydia polypeptide. In some embodiments, a chlamydia antigen comprises a portion or portions of a full-length chlamydia polypeptide. In some embodiments, a chlamydia antigen comprises a chlamydia polypeptide that lacks a signal sequence and/or trans-membrane domain. In some embodiments, a chlamydia antigen comprises a mixture of full-length chlamydia polypeptide and fragments resulting from processing, or partial processing, of a signal sequence by an expression host, e.g., E. coli, an insect cell line (e.g. the baculovirus expression system), or a mammalian (e.g., human or Chinese Hamster Ovary) cell line. As used herein, the terms “portion” and “fragment”, or grammatical equivalents, are used interchangeably.

In some embodiments, an immunogenic composition comprises a CT062 polypeptide antigen. In some embodiments, a CT062 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of a CT062 polypeptide sequence. In some embodiments, a CT062 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1. In some embodiments, a CT062 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1.

In some embodiments, an immunogenic composition comprises a CT572 polypeptide antigen. In some embodiments, a CT572 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of a CT572 polypeptide sequence. In some embodiments, a CT572 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of the sequence shown in SEQ ID NO:3. In some embodiments, a CT572 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of the sequence shown in SEQ ID NO:3.

In some embodiments, an immunogenic composition comprises a CT043 polypeptide antigen. In some embodiments, a CT043 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of a CT043 polypeptide sequence. In some embodiments, a CT043 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of the sequence shown in SEQ ID NO:5. In some embodiments, a CT043 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of the sequence shown in SEQ ID NO:5.

In some embodiments, an immunogenic composition comprises a CT570 polypeptide antigen. In some embodiments, a CT570 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of a CT570 polypeptide sequence. In some embodiments, a CT570 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of the sequence shown in SEQ ID NO:7. In some embodiments, a CT570 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of the sequence shown in SEQ ID NO:7.

In some embodiments, an immunogenic composition comprises a CT177 polypeptide antigen. In some embodiments, a CT177 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of a CT177 polypeptide sequence. In some embodiments, a CT177 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of the sequence shown in SEQ ID NO:9. In some embodiments, a CT177 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of the sequence shown in SEQ ID NO:9.

In some embodiments, an immunogenic composition comprises a CT725 polypeptide antigen. In some embodiments, a CT725 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of a CT725 polypeptide sequence. In some embodiments, a CT725 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of the sequence shown in SEQ ID NO:11. In some embodiments, a CT725 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of the sequence shown in SEQ ID NO:11.

In some embodiments, an immunogenic composition comprises a CT067 polypeptide antigen. In some embodiments, a CT067 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of a CT067 polypeptide sequence. In some embodiments, a CT067 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of the sequence shown in SEQ ID NO:23. In some embodiments, a CT067 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of the sequence shown in SEQ ID NO:23.

In some embodiments, an immunogenic composition comprises a CT476 polypeptide antigen. In some embodiments, a CT476 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of a CT476 polypeptide sequence. In some embodiments, a CT476 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of the sequence shown in SEQ ID NO:63. In some embodiments, a CT476 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of the sequence shown in SEQ ID NO:63.

In some embodiments, an immunogenic composition comprises a p6 polypeptide antigen from the cryptic plasmid of chlamydia. In some embodiments, a p6 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of a p6 polypeptide sequence. In some embodiments, a p6 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of the sequence shown in SEQ ID NO:65. In some embodiments, a p6 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of the sequence shown in SEQ ID NO:65.

In some embodiments, an immunogenic composition comprises a CT310 polypeptide antigen. In some embodiments, a CT310 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, 180, 190, or 200 consecutive amino acids of a CT310 polypeptide sequence. In some embodiments, a CT310 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, 180, 190, or 200 consecutive amino acids of the sequence shown in SEQ ID NO:67. In some embodiments, a CT310 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, 180, 190, or 200 consecutive amino acids of the sequence shown in SEQ ID NO:67.

In some embodiments, an immunogenic composition comprises a CT638 polypeptide antigen. In some embodiments, a CT638 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, 200, or 250 consecutive amino acids of a CT638 polypeptide sequence. In some embodiments, a CT638 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, 200, or 250 consecutive amino acids of the sequence shown in SEQ ID NO:69. In some embodiments, a CT638 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 100, 150, 200, or 250 consecutive amino acids of the sequence shown in SEQ ID NO:69.

TABLE 1

| Chlamydia | Protein | DNA | | GenBank |
|---|---|---|---|---|
| Antigen | SEQ ID | SEQ ID | Gene ID | Accession No. |
| Name | NO: | NO: | No. | NC_000117 |
| CT062 | 1 | 2 | 884058 | NP_219565.1 |
| CT572 | 3 | 4 | 884363 | NP_220087.1 |
| CT043 | 5 | 6 | 884043 | NP_219546.1 |
| CT570 | 7 | 8 | 884346 | NP_220085.1 |
| CT177 | 9 | 10 | 884953 | NP_219681.1 |
| CT725 | 11 | 12 | 884517 | NP_220244.1 |
| CT067 | 23 | 24 | 884065 | NP_219570.1 |
| CT476 | 63 | 64 | 884252 | NP_219989.1 |

TABLE 2

| Chlamydia | Protein | DNA | | GenBank |
|---|---|---|---|---|
| Antigen | SEQ ID | SEQ ID | Gene ID | Accession No. |
| Name | NO: | NO: | No. | NC_000117 |
| CT856 | 13 | 14 | 884657 | NP_220378.1 |
| CT757 | 15 | 16 | 884554 | NP_220276.1 |
| CT564 | 17 | 18 | 884347 | NP_220079.1 |
| CT703 | 19 | 20 | 884507 | NP_220222.1 |
| p1-ORF7 | 21 | 22 | 144463 | AAA91567.1 |
| CT037 | 25 | 26 | 884081 | NP_219539.1 |
| CT252 | 27 | 28 | 884872 | NP_219757.1 |
| CT064 | 29 | 30 | 884077 | NP_219567.1 |
| CT137 | 31 | 32 | 884086 | NP_219640.1 |
| CT204 | 33 | 34 | 884923 | NP_219708.1 |
| CT634 | 35 | 36 | 884415 | NP_220151.1 |
| CT635 | 37 | 38 | 884441 | NP_220152.1 |
| CT366 | 39 | 40 | 884747 | NP_219875.1 |
| CT140 | 41 | 42 | 884136 | NP_219643.1 |
| CT142 | 43 | 44 | 884051 | NP_219645.1 |
| CT242 | 45 | 46 | 884883 | NP_219747.1 |
| CT843 | 47 | 48 | 884645 | NP_220364.1 |
| CT328 | 49 | 50 | 884786 | NP_219835.1 |
| CT188 | 51 | 52 | 884942 | NP_219692.1 |
| CT578 | 53 | 54 | 884355 | NP_220093.1 |
| CT724 | 55 | 56 | 884515 | NP_220243.1 |
| CT722 | 57 | 58 | 884513 | NP_220241.1 |
| CT732 | 59 | 60 | 884527 | NP_220251.1 |
| CT788 | 61 | 62 | 884590 | NP_220307.1 |

TABLE 3

| Chlamydia | Protein | DNA | | |
|---|---|---|---|---|
| Antigen | SEQ ID | SEQ ID | Gene ID | GenBank |
| Name | NO: | NO: | No. | Accession No. |
| p6 | 65 | 66 | 144468 | AAA91572.1 |
| CT310 | 67 | 68 | 884815 | NP_219815.1 |
| CT638 | 69 | 70 | 884420 | NP_220155.1 |
| CT172 | 71 | 72 | 884959 | NP_219675.1 |
| CT443 | 73 | 74 | 884223 | NP_219955.1 |
| CT525 | 75 | 76 | 884305 | NP_220040.1 |
| CT606 | 77 | 78 | 884386 | NP_220122.1 |
| CT648 | 79 | 80 | 884431 | NP_220166.1 |
| CT870 | 81 | 82 | 884672 | NP_220392.1 |

In some embodiments, an immunogenic composition comprises two or more isolated chlamydia antigens. In some embodiments, the two or more isolated chlamydia antigens comprise two or more of a polypeptide antigen selected from Table 1. In some embodiments, the two or more isolated chlamydia antigens comprise three or more of a polypeptide antigen selected from Table 1. In some embodiments, the two or more isolated chlamydia antigens comprise four or more of a polypeptide antigen selected from Table 1. In some embodiments, the two or more isolated chlamydia antigens comprise five, six, seven or more of a polypeptide antigen selected from Table 1. In some embodiments, the two or more isolated chlamydia antigens comprise eight polypeptide antigens selected from Table 1.

Inventive chlamydia antigens described herein may be used in conjunction with other chlamydia antigens such as those known in the art. In some embodiments, an immunogenic composition comprises two or more isolated chlamydia antigens, wherein the two or more isolated chlamydia antigens comprise (a) one or more chlamydia polypeptide antigens selected from Table 1; and (b) one or more chlamydia polypeptide antigens selected from Table 2. In some embodiments, an immunogenic composition comprises two or more isolated chlamydia antigens, wherein the two or more isolated chlamydia antigens comprise (a) one or more chlamydia polypeptide antigens selected from Table 1; and (b) one or more chlamydia polypeptide antigens selected from Table 3. In some embodiments, an immunogenic composition comprises two or more isolated chlamydia antigens, wherein the two or more isolated chlamydia antigens comprise (a) one or more chlamydia polypeptide antigens selected from Table 2; and (b) one or more chlamydia polypeptide antigens selected from Table 3. In some embodiments, an immunogenic composition comprises three or more isolated chlamydia antigens, wherein the three or more isolated chlamydia antigens comprise (a) one or more chlamydia polypeptide antigens selected from Table 1; (b) one or more chlamydia polypeptide antigens selected from Table 2; and (c) one or more chlamydia polypeptide antigens selected from Table 3.

In some embodiments, an immunogenic composition comprises an isolated chlamydia polypeptide antigen selected from Table 2.

In some embodiments, an immunogenic composition comprises an isolated chlamydia polypeptide antigen selected from Table 3.

In some embodiments, an immunogenic composition comprises two, three, four, five or more isolated chlamydia polypeptide antigens selected from Table 2.

In some embodiments, an immunogenic composition comprises two, three, four, five or more isolated chlamydia polypeptide antigens selected from Table 3.

In some embodiments, a chlamydia antigen is fused to a heterologous polypeptide (e.g., an epitope tag).

In some embodiments, an immunogenic composition comprising a chlamydia antigen includes a pharmaceutically acceptable excipient.

In some embodiments, an immunogenic composition comprising a chlamydia antigen includes an adjuvant. In some embodiments, an immunogenic composition includes a mineral-containing adjuvant. In some embodiments, the mineral-containing adjuvant includes aluminum hydroxide. In some embodiments, an immunogenic composition includes an adjuvant comprising an immunomodulatory oligonucleotide. In some embodiments, an immunogenic composition includes IC31™ adjuvant (Intercell AG). In some embodiments, an immunogenic composition includes an adjuvant comprising a toxin. In some embodiments, an immunogenic composition includes an adjuvant comprising an endotoxin. In some embodiments, an immunogenic composition includes an adjuvant comprising a muramyl dipeptide. In some embodiments, an immunogenic composition includes an adjuvant comprising an oil emulsion. In some embodiments, an immunogenic composition includes an adjuvant comprising a saponin. In some embodiments, an immunogenic composition includes an adjuvant comprising an immune stimulating complex (ISCOM). In some embodiments, an immunogenic composition includes an adjuvant comprising a nonionic block copolymer. In some embodiments, an immunogenic composition includes virus-like particles (VLPs). In some embodiments, an immunogenic composition includes replicons. In some embodiments, an immunogenic composition includes an adjuvant comprising lipososmes. In some embodiments, an immunogenic composition includes an adjuvant comprising microparticles. In some embodiments, an immunogenic composition includes an adjuvant comprising biodegradable microspheres. In some embodiments, an immunogenic composition includes an adjuvant comprising a cytokine. In some embodiments, an immunogenic composition includes an adjuvant comprising a lipopeptide.

In some embodiments, an immunogenic composition elicits an immune response to Chlamydia trachomatis. In some embodiments, an immunogenic composition elicits a T cell-mediated immune response to a chlamydia antigen (e.g., a CD4⁺ T cell-mediated immune response and/or a CD8⁺ T cell-mediated immune response). In some embodiments, an immunogenic composition elicits a Th1 T cell response. In some embodiments, an immunogenic composition elicits a Th17 T cell response. In some embodiments, an immunogenic composition elicits IFN-γ secretion by antigen-specific T cells. In some embodiments, an immunogenic composition elicits a cytotoxic T cell response. In some embodiments, an immunogenic composition elicits an antibody response (e.g., an IgG response, and/or an IgA response). In some embodiments, an immunogenic composition elicits a B cell-mediated immune response. In some embodiments, an immunogenic composition elicits both a T cell- and a B cell-mediated response. In some embodiments, an immunogenic composition elicits an innate immune response.

In another aspect, the invention provides methods for eliciting an immune response against chlamydia in a mammal. The methods include, for example, administering to the mammal an immunogenic composition comprising an isolated chlamydia polypeptide antigen selected from Table 1, Table 2, or Table 3, or combinations thereof, e.g., an immunogenic composition described herein.

In some embodiments, a method elicits an immune response against Chlamydia trachomatis. In some embodiments, a method elicits a T cell response to a chlamydia antigen (e.g., a CD4⁺ T cell mediated immune response and/or a CD8⁺ T cell mediated immune response). In some embodiments, a method elicits a Th1 T cell response. In some embodiments, a method elicits a Th17 T cell response. In some embodiments, a method elicits IFN-γ secretion by antigen-specific T cells. In some embodiments, a method elicits an antibody response (e.g., an IgG response, and/or an IgA response). In some embodiments, a method elicits a cytotoxic T cell response. In some embodiments, a method elicits a B cell-mediated immune response. In some embodiments, a method elicits both a T cell- and a B cell-mediated response. In some embodiments, a method elicits an innate immune response.

In some embodiments, a method reduces the incidence of chlamydia infection in subjects administered the composition. In some embodiments, a method reduces the likelihood of lower tract infection by a chlamydia organism. In some embodiments, a method reduces the likelihood of upper tract infection by a chlamydia organism. In some embodiments, a method reduces the likelihood of chronic infection by a chlamydia organism. In some embodiments, a method reduces the likelihood of suffering from pelvic inflammatory disease due to a chlamydia infection. In some embodiments, a method reduces the likelihood of infertility subsequent to a chlamydia infection.

In some embodiments of a method, an immunogenic composition is administered to the mammal at least two times (e.g., two, three, four, or five times).

In some embodiments, an immunogenic composition administered after a first administration (i.e., as a boost) differs from the composition administered initially, e.g., the composition includes a different chlamydia antigen or a different subset of chlamydia antigens, or a different chlamydia antigen substance (polypeptide or nucleic acid encoding same), or a different dose of antigen, or a different adjuvant, or a different dose of adjuvant. In some embodiments, a boost is administered by a different route than a previous administration.

In some embodiments, the mammal is at risk for infection with Chlamydia trachomatis. In some embodiments, the mammal is infected with Chlamydia trachomatis. In some embodiments, the mammal is a female. In some embodiments, the mammal is a human.

In some embodiments, an immunogenic composition administered in a method comprises an adjuvant. In some embodiments, an adjuvant is a mineral-containing adjuvant. In some embodiments, an immunogenic composition administered in a method comprises a pharmaceutically acceptable excipient.

In some embodiments, an immunogenic composition comprises an adjuvant. In some embodiments, an immunogenic composition includes a mineral-containing adjuvant. In some embodiments, a mineral-containing adjuvant includes aluminum hydroxide. In some embodiments, an immunogenic composition includes an adjuvant comprising an immunomodulatory oligonucleotide. In some embodiments, an immunogenic composition includes IC31™ adjuvant (Intercell AG). In some embodiments, an immunogenic composition includes an adjuvant comprising a toxin. In some embodiments, an immunogenic composition includes an adjuvant comprising an endotoxin. In some embodiments, an immunogenic composition includes an adjuvant comprising a muramyl dipeptide. In some embodiments, an immunogenic composition includes an adjuvant comprising an oil emulsion. In some embodiments, an immunogenic composition includes an adjuvant comprising a saponin. In some embodiments, an immunogenic composition includes an adjuvant comprising an immune stimulating complex (ISCOM). In some embodiments, an immunogenic composition includes an adjuvant comprising a nonionic block copolymer. In some embodiments, an immunogenic composition includes virus-like particles (VLPs). In some embodiments, an immunogenic composition includes replicons. In some embodiments, an immunogenic composition includes an adjuvant comprising lipososmes. In some embodiments, an immunogenic composition includes an adjuvant comprising microparticles. In some embodiments, an immunogenic composition includes an adjuvant comprising biodegradable microspheres. In some embodiments, an immunogenic composition includes an adjuvant comprising a cytokine. In some embodiments, an immunogenic composition includes an adjuvant comprising a lipopeptide.

In some embodiments of provided methods, an immunogenic composition comprises a CT062 polypeptide antigen. In some embodiments, a CT062 polypeptide antigen comprises 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of a CT062 polypeptide sequence. In some embodiments, a CT062 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1. In some embodiments, a CT062 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1.

In some embodiments of provided methods, an immunogenic composition comprises a CT572 polypeptide antigen. In some embodiments, a CT572 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of a CT572 polypeptide sequence. In some embodiments, a CT572 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of the sequence shown in SEQ ID NO:3. In some embodiments, a CT572 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of the sequence shown in SEQ ID NO:3.

In some embodiments of provided methods, an immunogenic composition comprises a CT043 polypeptide antigen. In some embodiments, a CT043 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of a CT043 polypeptide sequence. In some embodiments, a CT043 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of the sequence shown in SEQ ID NO:5. In some embodiments, a CT043 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of the sequence shown in SEQ ID NO:5.

In some embodiments of provided methods, an immunogenic composition comprises a CT570 polypeptide antigen. In some embodiments, a CT570 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of a CT570 polypeptide sequence. In some embodiments, a CT570 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of the sequence shown in SEQ ID NO:7. In some embodiments, a CT570 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of the sequence shown in SEQ ID NO:7.

In some embodiments of provided methods, an immunogenic composition comprises a CT177 polypeptide antigen. In some embodiments, a CT177 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of a CT177 polypeptide sequence. In some embodiments, a CT177 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of the sequence shown in SEQ ID NO:9. In some embodiments, a CT177 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of the sequence shown in SEQ ID NO:9.

In some embodiments of provided methods, an immunogenic composition comprises a CT725 polypeptide antigen. In some embodiments, a CT725 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of a CT725 polypeptide sequence. In some embodiments, a CT725 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of the sequence shown in SEQ ID NO:11. In some embodiments, a CT725 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of the sequence shown in SEQ ID NO:11.

In some embodiments of provided methods, an immunogenic composition comprises a CT067 polypeptide antigen. In some embodiments, a CT067 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of a CT067 polypeptide sequence. In some embodiments, a CT067 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of the sequence shown in SEQ ID NO:23. In some embodiments, a CT067 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of the sequence shown in SEQ ID NO:23.

In some embodiments of provided methods, an immunogenic composition comprises a CT476 polypeptide antigen. In some embodiments, a CT476 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of a CT476 polypeptide sequence. In some embodiments, a CT476 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of the sequence shown in SEQ ID NO:63. In some embodiments, a CT476 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of the sequence shown in SEQ ID NO:63.

In some embodiments of provided methods, an immunogenic composition comprises a p6 polypeptide antigen from the cryptic plasmid of chlamydia. In some embodiments, a p6 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of a p6 polypeptide sequence. In some embodiments, a p6 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of the sequence shown in SEQ ID NO:65. In some embodiments, a p6 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of the sequence shown in SEQ ID NO:65.

In some embodiments of provided methods, an immunogenic composition comprises a CT310 polypeptide antigen. In some embodiments, a CT310 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, 180, 190, or 200 consecutive amino acids of a CT310 polypeptide sequence. In some embodiments, a CT310 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, 180, 190, or 200 consecutive amino acids of the sequence shown in SEQ ID NO:67. In some embodiments, a CT310 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, 180, 190, or 200 consecutive amino acids of the sequence shown in SEQ ID NO:67.

In some embodiments of provided methods, an immunogenic composition comprises a CT638 polypeptide antigen. In some embodiments, a CT638 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, 200, or 250 consecutive amino acids of a CT638 polypeptide sequence. In some embodiments, a CT638 polypeptide antigen comprises at least 7, 8, 9, 10, 11, 12, 13, 14, 15, 16, 17, 18, 19, 20, 25, 30, 35, 45, 50, 60, 65, 70, 75, 80, 85, 90, 100, 150, 200, or 250 consecutive amino acids of the sequence shown in SEQ ID NO:69. In some embodiments, a CT638 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 100, 150, 200, or 250 consecutive amino acids of the sequence shown in SEQ ID NO:69.

In some embodiments of provided methods, an immunogenic composition comprises two or more isolated chlamydia antigens. In some embodiments, the two or more isolated chlamydia antigens comprise two or more of a polypeptide antigen selected from Table 1. In some embodiments, the two or more isolated chlamydia antigens comprise three or more of a polypeptide antigen selected from Table 1. In some embodiments, the two or more isolated chlamydia antigens comprise four or more of a polypeptide antigen selected from Table 1. In some embodiments, the two or more isolated chlamydia antigens comprise five, six, seven or more of a polypeptide antigen selected from Table 1. In some embodiments, the two or more isolated chlamydia antigens comprise eight polypeptide antigens selected from Table 1.

In some embodiments of provided methods, inventive chlamydia antigens described herein are used in conjunction with one or more additional chlamydia antigens including those known in the art. In some embodiments, an immunogenic composition suitable for a method of the invention comprises two or more isolated chlamydia antigens, wherein the two or more isolated chlamydia antigens comprise (a) one or more chlamydia polypeptide antigens selected from Table 1; and (b) one or more chlamydia polypeptide antigens selected from Table 2. In some embodiments of provided methods, an immunogenic composition comprises two or more isolated chlamydia antigens, wherein the two or more isolated chlamydia antigens comprise (a) one or more chlamydia polypeptide antigens selected from Table 1; and (b) one or more chlamydia polypeptide antigens selected from Table 3. In some embodiments, an immunogenic composition comprises two or more isolated chlamydia antigens, wherein the two or more isolated chlamydia antigens comprise (a) one or more chlamydia polypeptide antigens selected from Table 2; and (b) one or more chlamydia polypeptide antigens selected from Table 3. In some embodiments of provided methods, an immunogenic composition comprises three or more isolated chlamydia antigens, wherein the three or more isolated chlamydia antigens comprise (a) one or more chlamydia polypeptide antigens selected from Table 1; (b) one or more chlamydia polypeptide antigens selected from Table 2; and (c) one or more chlamydia polypeptide antigens selected from Table 3.

In some embodiments of provided methods, an immunogenic composition comprises an isolated chlamydia polypeptide antigen selected from Table 2.

In some embodiments of provided methods, an immunogenic composition comprises an isolated chlamydia polypeptide antigen selected from Table 3.

In some embodiments of provided methods, an immunogenic composition comprises two, three, four, five or more isolated chlamydia polypeptide antigens selected from Table 2.

In some embodiments of provided methods, an immunogenic composition comprises two, three, four, five or more isolated chlamydia polypeptide antigens selected from Table 3.

In some embodiments, an immunogenic composition comprises a chlamydia antigen and an antigen from a different infectious agent. In some embodiments, an immunogenic composition comprises a chlamydia polypeptide antigen selected from Table 1, Table 2, Table 3, or combinations thereof; and an antigen from a papillomavirus (e.g., a human papillomavirus). In some embodiments, an immunogenic composition comprises a chlamydia polypeptide antigen selected from Table 1, Table 2, Table 3, or combinations thereof; and an antigen from a herpesvirus (e.g., herpes simplex virus-2). In some embodiments, an immunogenic composition comprises a chlamydia polypeptide antigen selected from Table 1, Table 2, Table 3, or combinations thereof; and an antigen from Neissiria gonorrhoeae.). In some embodiments, an immunogenic composition comprises a chlamydia polypeptide antigen selected from Table 1, Table 2, Table 3, or combinations thereof; and an antigen from Candida albicans. In some embodiments, an immunogenic composition comprises a chlamydia polypeptide antigen selected from Table 1, Table 2, Table 3, or combinations thereof; and an antigen from one or more of a papillomavirus, a herpesvirus (e.g., herpes simplex virus-2), Neissiria gonorrhoeae, and Candida albicans

In another aspect, the invention provides isolated nucleic acids comprising a nucleotide sequence encoding a chlamydia antigen described herein. In some embodiments, the invention provides isolated nucleic acids comprising a nucleotide sequence encoding a chlamydia antigen selected from Table 1, Table 2, Table 3, or combinations thereof. In some embodiments, a nucleic acid further comprises a nucleotide sequence encoding a heterologous peptide fused to the chlamydia antigen.

The invention also provides compositions including nucleic acids encoding a chlamydia antigen as described herein. In some embodiments, a composition includes an isolated nucleic acid comprising a nucleotide sequence encoding a chlamydia antigen selected from Table 1, Table 2, Table 3, or combinations thereof, and further comprises a pharmaceutically acceptable excipient. In some embodiments, a composition further comprises an adjuvant.

In still another aspect, the invention provides methods for eliciting an immune response against chlamydia in a mammal based on nucleic acids described herein. In some embodiments, the invention provides methods for eliciting an immune response against chlamydia in a mammal by administering to the mammal a composition comprising a nucleic acid, wherein the nucleic acid comprises a nucleotide sequence encoding a chlamydia antigen selected from Table 1, Table 2, Table 3, or combinations thereof.

In another aspect, the invention provides methods for characterizing and/or detecting an immune response to a chlamydia antigen in a subject (e.g., a chlamydia polypeptide antigen selected from Table 1, Table 2, Table 3, or combinations thereof). In some embodiments, an immune response in a naïve subject is characterized. In some embodiments, an immune response in a subject infected, or suspected of having been infected, with chlamydia is characterized. In some embodiments, an immune response in a subject administered an immunogenic composition comprising a chlamydia antigen (e.g., an immunogenic composition described herein) is characterized. In some embodiments, an antibody response is characterized. In some embodiments, a B cell response is characterized. In some embodiments, a T cell response is characterized. In some embodiments, IFN-γ secretion by antigen-specific T cells is characterized. In some embodiments, a Th1 T cell response is characterized. In some embodiments, a Th17 T cell response is characterized. In some embodiments, a cytotoxic T cell response is characterized. In some embodiments, both a T cell and a B cell response are characterized. In some embodiments, an innate immune response is characterized.

The invention further provides methods of preparing compositions including chlamydia antigens, and antibodies that specifically bind to chlamydia antigens.

Compositions and methods described herein can be used for the prophylaxis and/or treatment of any chlamydial disease, disorder, and/or condition, e.g., any of urethritis, cervicitis, pharyngitis, proctitis, epididymitis, prostatitis, pelvic inflammatory disease, and trachoma, due to a chlamydia infection. In some embodiments, an immunogenic composition described herein reduces risk of infection by, and/or treats, alleviates, ameliorates, relieves, delays onset of, inhibits progression of, reduces severity of, and/or reduces incidence of one or more symptoms or features of a chlamydial disease, disorder, and/or condition. In some embodiments, the prophylaxis and/or treatment of chlamydia infection comprises administering a therapeutically effective amount of an immunogenic composition comprising a novel chlamydial antigen described herein to a subject in need thereof, in such amounts and for such time as is necessary to achieve the desired result. In certain embodiments of the present invention a “therapeutically effective amount” of an inventive immunogenic composition is that amount effective for treating, alleviating, ameliorating, relieving, delaying onset of, inhibiting progression of, reducing severity of, and/or reducing incidence of one or more symptoms or features of chlamydia infection.

In some embodiments, inventive prophylactic, prognostic and/or therapeutic protocols involve administering a therapeutically effective amount of one or more immunogenic compositions comprising a novel chlamydia antigen to a subject such that an immune response is stimulated in one or both of T cells and B cells.

The present invention provides novel immunogenic compositions comprising a therapeutically effective amount of one or more chlamydia antigens (e.g., one or more of a polypeptide antigen selected from Table 1, Table 2, Table 3, or combinations thereof) and one or more pharmaceutically acceptable excipients. In some embodiments, the present invention provides for pharmaceutical compositions comprising an immunogenic composition as described herein. In accordance with some embodiments, a method of administering a pharmaceutical composition comprising inventive compositions to a subject (e.g. human, e.g., a child, adolescent, or young adult) in need thereof is provided.

In some embodiments, a therapeutically effective amount of an immunogenic composition is delivered to a patient and/or animal prior to, simultaneously with, and/or after diagnosis with a chlamydial disease, disorder, and/or condition. In some embodiments, a therapeutic amount of an inventive immunogenic composition is delivered to a patient and/or animal prior to, simultaneously with, and/or after onset of symptoms of a chlamydial disease, disorder, and/or condition.

In some embodiments, immunogenic compositions of the present invention are administered by any of a variety of routes, including oral, intramuscular, subcutaneous, transdermal, interdermal, rectal, intravaginal, mucosal, nasal, buccal, enteral, sublingual; by intratracheal instillation, bronchial instillation, and/or inhalation; and/or as an oral spray, nasal spray, and/or aerosol. In some embodiments, immunogenic compositions of the present invention are administered by a variety of routes, including intravenous, intra-arterial, intramedullary, intrathecal, intraventricular, transdermal, intraperitoneal, topical (as by powders, ointments, creams, and/or drops), transdermal, or by intratracheal instillation.

In certain embodiments, an immunogenic composition may be administered in combination with one or more additional therapeutic agents which treat the symptoms of chlamydia infection (e.g., with an antibiotic such as an erythromycin or a tetracycline).

The invention provides a variety of kits comprising one or more of the immunogenic compositions of the invention. For example, the invention provides a kit comprising an immunogenic composition comprising a chlamydia antigen, or a nucleic acid encoding the antigen, wherein the antigen is selected from Table 1, Table 2, Table 3, or combinations thereof; and instructions for use. A kit may comprise multiple different chlamydia antigens. A kit may comprise any of a number of additional components or reagents in any combination. According to certain embodiments of the invention, a kit may include, for example, (i) a chlamydia polypeptide antigen selected from Table 1, Table 2, Table 3, or combinations thereof; (ii) an adjuvant; and (iii) instructions for administering a composition including the chlamydia antigen and the adjuvant to a subject in need thereof.

This application refers to various issued patents, published patent applications, journal articles, database entries containing amino acid and nucleic acid sequence information, and other publications, all of which are incorporated herein by reference.

### BRIEF DESCRIPTION OF THE DRAWING

The Figures described below, that together make up the Drawing, are for illustration purposes only, not for limitation.

FIGS. 1, 2, and 3 depict exemplary graphs illustrating the frequency with which identified antigens were recognized by human donor CD4⁺ and CD8⁺ T cells, respectively. Human donors were women with documented Chlamydia trachomatis exposure or a clinical history of genital infection. Donors were classified as “protected” if they were repeatedly exposed to the bacteria but not infected, or if they became infected but cleared their infection without medical intervention. Donors were classified as “unprotected” if they were persistently infected or if their infections progressed to more severe complications such as pelvic inflammatory disease. Based on evaluation of negative controls and normalization for donor and plate variation, a donor was classified as a “responder” if the fold ratio of the response value over negative control was greater than 1.63 (CD4⁺) or 1.66 (CD8⁺). Percent responders >10% indicated a higher number of responders than due to chance alone. Statistical significance was reached when the percent responders was >15% (all donors, including negative controls), or approximately 19% (protected and unprotected donors). FIG. 1 depicts an exemplary result for protected and unprotected donors. FIG. 2 depicts another exemplary result for protected and unprotected donors. Four C. trachomatis proteins induced CD4⁺ or CD8⁺ T cell responses (two clones each, respectively) with statistically greater frequency in protected compared to unprotected donors, with a p-value of 0.05. An additional 16 clones induced CD8⁺ T cell responses and 6 clones induced CD4⁺ T cell responses with greater frequency in protected donors, with a p-value of 0.1. Antigens that are represented with greater frequency in donors who were clinically protected from their infection are correlated with protective immunity and the best candidates for vaccine formulation. FIG. 3 depicts an exemplary result illustrating CD4⁺, CD8⁺, and combined T cell responses for all donors (protected and unprotected). Antigens represented at the highest overall frequency, whether or not represented at statistically higher frequency in protected donors, are also attractive candidates for vaccine, diagnostic and prognostic applications.

FIG. 4 depicts an exemplary result illustrating the frequency with which chlamydia antigens were bound by IgG present in donor sera, i.e. have elicited a donor B cell response. The left side of the panel displays chlamydia antigens detected by IgG with overall highest frequency across all donors (protected and unprotected). The right side of the panel displays chlamydia antigens detected by IgG with statistically greater frequency in protected donors as compared to unprotected donors.

FIG. 5 depicts an exemplary result illustrating IFN-γ levels induced ex vivo in CD4⁺ and CD8⁺ T cells from mice immunized with an identified chlamydia protein antigen, following challenge with the same antigen. FIG. 5A depicts an exemplary result illustrating antigens that were originally identified through T cell responses. FIG. 5B depicts an exemplary result illustrating antigens that were originally identified through B cell responses, demonstrating that these antigens can in some cases also elicit robust T cell responses.

FIG. 6 depicts an exemplary result illustrating IgG antibody titers against each chlamydia antigen, following immunization with the same antigen. Exemplary results shown in the left side of the panel illustrate that antigens originally identified through T cell responses (e.g. FIGS. 1, 2 and 3) can in some cases also elicit robust B cell responses.

FIG. 7 depicts an exemplary result illustrating reduction of ectocervical chlamydia burden in mice immunized with identified chlamydia protein antigens and subsequently intravaginally infected with Chlamydia trachomatis. FIG. 7A depicts an exemplary result for representative chlamydia protein antigens CT062, CT043, and for the combination CT062+CT043. FIG. 7B depicts an exemplary result for representative chlamydia protein antigen combination CT638+CT476.

FIG. 8 depicts an exemplary result illustrating reduction of upper reproductive tract chlamydia burden in mice immunized with the identified chlamydia protein antigens and subsequently intravaginally infected with Chlamydia trachomatis. FIG. 8A depicts an exemplary result for representative chlamydia protein antigens CT062, CT043, and for the combination CT062+CT043. UVEB indicates responses from mice immunized with the positive control, UV-inactivated whole Chlamydia trachomatis elementary bodies. FIG. 8B depicts an exemplary result for representative chlamydia protein antigens CT067, CT0788tm, and CT328.

FIG. 9 depicts an exemplary result illustrating induction of IFN-γ in CD4⁺ and CD8⁺ T cells harvested from the spleens of infected mice and stimulated with identified chlamydia protein antigens. Exemplary results illustrate that infection with Chlamydia trachomatis can prime T cells that are specific for the identified antigens, and that can be the target of protective T cells upon re-challenge.

### DEFINITIONS

In order for the present invention to be more readily understood, certain terms are first defined below. Additional definitions for the following terms and other terms are set forth throughout the specification.

Adjuvant: As used herein, the term “adjuvant” refers to an agent that alters (e.g., enhances) an immune response to an antigen. In some embodiments, an adjuvant is used to enhance an immune response to a peptide antigen administered to a subject. In some embodiments, an adjuvant is used to enhance an immune response to an antigen encoded by a nucleic acid administered to a subject.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

Antigen: The term “antigen”, as used herein, refers to a molecule (e.g., a polypeptide) that elicits a specific immune response. Antigen specific immunological responses, also known as adaptive immune responses, are mediated by lymphocytes (e.g., T cells, B cells) that express antigen receptors (e.g., T cell receptors, B cell receptors). In certain embodiments, an antigen is a T cell antigen, and elicits a cellular immune response. In certain embodiments, an antigen is a B cell antigen, and elicits a humoral (i.e., antibody) response. In certain embodiments, an antigen is both a T cell antigen and a B cell antigen. As used herein, the term “antigen” encompasses both a full-length polypeptide as well as a portion of the polypeptide, that represent immunogenic fragments (i.e., fragments that elicit an antigen specific T cell response, B cell response, or both) of such complete polypeptides. In some embodiments, antigen is a peptide epitope found within a polypeptide sequence (e.g., a peptide epitope bound by a Major Histocompatibility Complex (MHC) molecule (e.g., MHC class I, or MHC class II). Accordingly, peptides 5-15 amino acids in length, and longer polypeptides, e.g., having 60, 70, 75, 80, 85, 90, 100, 150, 200 250, or more amino acids, can be “antigens”. In one example, the present invention provides a CT062 polypeptide antigen. In some embodiments, a CT062 polypeptide antigen includes a full-length CT062 polypeptide amino acid sequence (e.g., a full-length CT062 polypeptide of SEQ ID NO:1). In some embodiments, a CT062 polypeptide antigen includes a portion of a CT062 polypeptide (e.g., a portion of the CT062 polypeptide of SEQ ID NO:1, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, or 400 contiguous amino acids of SEQ ID NO:1). In some embodiments, a CT062 polypeptide antigen contains one or more amino acid alterations (e.g., deletion, substitution, and/or insertion) from a naturally-occurring wild-type CT062 polypeptide sequence. For example, a CT062 polypeptide antigen may contain an amino acid sequence that is at least 60% (e.g., at least 65%, 70%, 75%, 80%, 85%, 90%, 95%, or 98%) identical to SEQ ID NO:1 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1). Alternatively, a CT062 polypeptide antigen may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids) of a sequence that is at least 60% (e.g., at least 65%, 70%, 75%, 80%, 85%, 90%, 95%, or 98%) identical to SEQ ID NO:1. CT062 polypeptide antigen is used as an example. This concept is applicable to other polypeptide antigen described herein including, but not limited to, CT572, CT043, CT570, CT177, CT725, CT067, CT476, p6, CT310, and CT638 polypeptide antigens.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Chlamydia antigen: As used herein, the term “chlamydia antigen” refers to an antigen that elicits an antigen specific immune response against any organism of the Chlamydia genus, such as a Chlamydia trachomatis organism, a Chlamydia psittaci organism, or a Chlamydia pneumoniae organism, a Chlamydia suis organism, a Chlamydia muridarum organism, etc. In some embodiments, a chlamydia antigen elicits an antigen specific immune response against chlamydia organisms of multiple species (e.g., two or three of Chlamydia trachomatis, Chlamydia psittaci, and Chlamydia pneumoniae). In some embodiments, a chlamydia antigen elicits an antigen specific immune response against chlamydia organisms of multiple serovars (e.g., one or more of serovars A, B, Ba, C, D, E, F, G, H, I, J, K, L1, L2, L3 of C. trachomatis). Chlamydia antigens include full-length polypeptides encoded by chlamydia genes, as well as immunogenic portions of the polypeptides.

Immunogenic composition: As used herein, the term “immunogenic composition” refers to a composition that includes a molecule that induces an immune response in a subject. In some embodiments, an immunogenic composition includes a polypeptide or peptide antigen. In some embodiments, an immunogenic composition includes a nucleic acid encoding a polypeptide or peptide antigen. An immunogenic composition can include molecules that induce an immune response against multiple antigens.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

Isolated: The term “isolated”, as used herein, means that the isolated entity has been separated from at least one component with which it was previously associated. When most other components have been removed, the isolated entity is “purified.” Isolation and/or purification and/or concentration may be performed using any techniques known in the art including, for example, chromatography, fractionation, precipitation, or other separation.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. The term “nucleotide sequence encoding an amino acid sequence” includes all nucleotide sequences that are degenerate versions of each other and/or encode the same amino acid sequence. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated.

Polypeptide: The term “polypeptide”, as used herein, generally has its art-recognized meaning of a polymer of at least three amino acids. However, the term is also used to refer to specific classes of antigen polypeptides, such as, for example, CT062 polypeptides, CT572 polypeptides, CT043 polypeptides, CT570 polypeptides, CT177 polypeptides, and CT725 polypeptides. For each such class, the present specification provides several examples of known sequences of such polypeptides. Those of ordinary skill in the art will appreciate, however, that the term “polypeptide”, as used herein to refer to “polypeptide antigen”, is intended to be sufficiently general as to encompass not only polypeptides having a sequence recited herein, but also to encompass polypeptides having a variation of the sequence that elicits an antigen-specific response to the polypeptide. For example, a “CT062 polypeptide” includes the CT062 polypeptide shown in SEQ ID NO:1, as well as polypeptides that have variations of a SEQ ID NO:1 sequence and that maintain the ability to elicit an antigen-specific response to a polypeptide of SEQ ID NO:1. Those of ordinary skill in the art understand that protein sequences generally tolerate some substitution without destroying immunogenicity and antigen specificity. Thus, any polypeptide that retains immunogenicity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented herein. See the definition of Antigen.

One example of an algorithm that is suitable for determining percent sequence identity and sequence similarity is the BLAST algorithm, which is described in Altschul et al., Nuc. Acids Res. 25:3389-3402, 1977. BLAST is used, with the parameters described herein, to determine percent sequence identity for the nucleic acids and proteins of the present disclosure. Software for performing BLAST analysis is publicly available through the National Center for Biotechnology Information (available at the following internet address: ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Subject: As used herein, the term “subject” or “patient” refers to any organism to which a composition of this invention may be administered, e.g., for experimental, diagnostic, and/or therapeutic purposes. Typical subjects include mammals such as mice, rats, rabbits, non-human primates, and humans.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, a disease, disorder, and/or condition is associated with a chlamydia infection (e.g., a C. trachomatis infection, a C. pneumoniae infection, or a C. psittaci infection). In some embodiments, an individual who is susceptible to a chlamydia infection may be exposed to a chlamydia microbe (e.g., by ingestion, inhalation, physical contact, etc.). In some embodiments, an individual who is susceptible to a chlamydia infection may be exposed to an individual who is infected with the microbe. In some embodiments, an individual who is susceptible to a chlamydia infection is one who is in a location where the microbe is prevalent (e.g., one who is traveling to a location where the microbe is prevalent). In some embodiments, an individual who is susceptible to a chlamydia infection is susceptible due to young age (e.g., a child, adolescent, or young adult). In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” means an amount of a therapeutic, prophylactic, and/or diagnostic agent (e.g., inventive immunogenic composition) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

Therapeutic agent: As used herein, the phrase “therapeutic agent” refers to any agent that, when administered to a subject, has a therapeutic, prophylactic, and/or diagnostic effect and/or elicits a desired biological and/or pharmacological effect.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an immunogenic composition (e.g., a vaccine) to a subject.

Vaccine: As used herein, the term “vaccine” refers to an entity comprising at least one immunogenic component (e.g., an immunogenic component which includes a peptide or protein, and/or an immunogenic component which includes a nucleic acid). In certain embodiments, a vaccine includes at least two immunogenic components. In some embodiments, a vaccine is capable of stimulating an immune response of both T cells and B cells. In some embodiments, any assay available in the art may be used to determine whether T cells and/or B cells have been stimulated. In some embodiments, T cell stimulation may be assayed by monitoring antigen-induced production of cytokines, antigen-induced proliferation of T cells, and/or antigen-induced changes in protein expression. In some embodiments, B cell stimulation may be assayed by monitoring antibody titers, antibody affinities, antibody performance in neutralization assays, class-switch recombination, affinity maturation of antigen-specific antibodies, development of memory B cells, development of long-lived plasma cells that can produce large amounts of high-affinity antibodies for extended periods of time, germinal center reactions, and/or antibody performance in neutralization assays. In some embodiments, a vaccine further includes at least one adjuvant that can help stimulate an immune response in T cells and/or B cells.

Wild-type: As used herein, the term “wild-type” refers to the typical or the most common form existing in nature.

### DETAILED DESCRIPTION OF CERTAIN EMBODIMENTS

Infection by Chlamydia trachomatis causes inflammation and damage to mucosal tissues, leading to pathologies such as urethritis, cervicitis, pharyngitis, proctitis, epididymitis, prostatitis, and trachoma, and infertility secondary to these pathologies. Chlamydia bacteria, which primarily infect epithelial cells, alternate between two developmental forms, the elementary body (EB) and reticulate body (RB). EB forms of chlamydia are infectious and invade host cells. After forming an inclusion within host cells, EB forms differentiate into RB forms which replicate for a period of time and differentiate back to EB forms. C. trachomatis species are categorized into serovars based on reactivity of patient sera to the major outer membrane protein (MOMP). Serovars A, B, Ba, and C are associated with infection of conjunctival epithelium. Serovars D-K are associated with urogenital tract infections. Serovars L1-L3 are associated with urogenital tract infection and a systemic condition, lymphogranuloma venereum.

Various arms of the adaptive immune system appear to play a role in responding to chlamydial infections. CD4⁺ T cell responses of the Th1 subtype have been shown to be important for clearance of chlamydia infections in an animal model (Morrison et al., Infect. Immun. 70:2741-2751, 2002). B cell responses are thought to contribute to protective immunity in humans and non-human primates (Brunham et al., Infect. Immun 39:1491-1494, 1983; Taylor et al., Invest. Ophthalmol. Vis. Sci 29:1847-1853, 1988). CD8⁺ T cells have lytic functions that are important for the control of intracellular pathogens. Chlamydia-specific CD8⁺ T cells have been isolated from infected humans, indicating a role for these cells in responding to chlamydia infections (Gervassi et al., J. Immunol. 171: 4278-4286, 2003).

The present invention provides chlamydia antigens, including, but not limited to, CT062 polypeptide antigens, CT572 polypeptide antigens, CT043 polypeptide antigens, CT570 polypeptide antigens, CT177 polypeptide antigens, CT725 polypeptide antigens, CT067 polypeptide antigens, CT476 polypeptide antigens, p6 polypeptide antigens, CT310 polypeptide antigens, and CT638 polypeptide antigens that are recognized by immune cells (e.g., T cells and/or B cells) of infected mammals. As described in the Examples herein, these antigens were discovered as targets of T cell- or B cell-mediated immunity in vivo. Accordingly, these antigens provide novel compositions for eliciting immune responses with the aim of eliciting beneficial immune responses, e.g., to protect against chlamydia infections and associated pathologies. These antigens also provide novel targets for characterizing chlamydia infections and immune responses to chlamydia infections.

CT062 polypeptides are cytoplasmic tyrosyl-tRNA synthetases in chlamydia organisms. Exemplary amino acid and nucleotide sequences from a full-length CT062 polypeptide of C. trachomatis are shown below as SEQ IDs NO:1 and 2. In some embodiments, a CT062 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of a CT062 polypeptide sequence, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1 or of a sequence at least 60% (e.g., at least 65%, 70%, 75%, 80%, 85%, 90%, 95%, or 98%) identical to SEQ ID NO:1. In some embodiments, a CT062 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO:1. In some embodiments, a CT062 polypeptide antigen is a full-length CT062 polypeptide (e.g., the antigen comprises the amino acid sequence of SEQ ID NO:1). In some embodiments, a CT062 polypeptide antigen lacks one or more trans-membrane domains (e.g., a CT062 polypeptide antigen lacks amino acids 55-74 of SEQ ID NO:1).

CT572 polypeptides are known as general secretion pathway proteins D. Exemplary amino acid and nucleotide sequences from a full-length CT572 polypeptide of C. trachomatis are shown below as SEQ IDs NO:3 and 4. In some embodiments, a CT572 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of a CT572 polypeptide sequence, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of the sequence shown in SEQ ID NO:3 or of a sequence at least 60% (e.g., at least 65%, 70%, 75%, 80%, 85%, 90%, 95%, or 98%) identical to SEQ ID NO:3. In some embodiments, a CT572 polypeptide antigen comprises an amino acid sequence that is at least 60% (e.g., at least 65%, 70%, 75%, 80%, 85%, 90%, 95%, or 98%) identical to at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, or 750 consecutive amino acids of the sequence shown in SEQ ID NO:3. In some embodiments, a CT572 polypeptide antigen is a full-length CT572 polypeptide (e.g., the antigen comprises the amino acid sequence of SEQ ID NO:3). In some embodiments, a CT572 polypeptide antigen lacks one or more trans-membrane domains and/or a signal sequence (e.g., a CT572 polypeptide antigen lacks amino acids 1-24 of SEQ ID NO:3).

Exemplary amino acid and nucleotide sequences from a full-length CT043 polypeptide of C. trachomatis are shown below as SEQ IDs NO:5 and 6. In some embodiments, a CT043 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of a CT043 polypeptide sequence, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of the sequence shown in SEQ ID NO:5 or of a sequence at least 60% (e.g., at least 65%, 70%, 75%, 80%, 85%, 90%, 95%, or 98%) identical to SEQ ID NO:5. In some embodiments, a CT043 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, or 160 consecutive amino acids of the sequence shown in SEQ ID NO:5. In some embodiments, a CT043 polypeptide antigen is a full-length CT043 polypeptide (e.g., the antigen comprises the amino acid sequence of SEQ ID NO:5). In some embodiments, a CT043 polypeptide antigen lacks one or more trans-membrane domains (e.g., a CT043 polypeptide antigen lacks amino acids 75-93 of SEQ ID NO:5).

CT570 polypeptides are known as general secretion pathway proteins F. Exemplary amino acid and nucleotide sequences from a full-length CT570 polypeptide of C. trachomatis are shown below as SEQ IDs NO:7 and 8. In some embodiments, a CT570 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of a CT570 polypeptide sequence, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of the sequence shown in SEQ ID NO:7 or of a sequence at least 60% (e.g., at least 65%, 70%, 75%, 80%, 85%, 90%, 95%, or 98%) identical to SEQ ID NO:7. In some embodiments, a CT570 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 350 consecutive amino acids of the sequence shown in SEQ ID NO:7. In some embodiments, a CT570 polypeptide antigen is a full-length CT570 polypeptide (e.g., the antigen comprises the amino acid sequence of SEQ ID NO:7). In some embodiments, a CT570 polypeptide antigen lacks one or more trans-membrane domains (e.g., a CT570 polypeptide antigen lacks amino acids 164-182 and/or 211-230 and/or 363-382 of SEQ ID NO:7).

CT177 polypeptides are disulfide bond chaperone proteins. Exemplary amino acid and nucleotide sequences from a full-length CT177 polypeptide of C. trachomatis are shown below as SEQ IDs NO:9 and 10. In some embodiments, a CT177 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of a CT177 polypeptide sequence, e.g., at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of the sequence shown in SEQ ID NO:9 or of a sequence at least 60% (e.g., at least 65%, 70%, 75%, 80%, 85%, 90%, 95%, or 98%) identical to SEQ ID NO:9. In some embodiments, a CT177 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 100, 150, or 200 consecutive amino acids of the sequence shown in SEQ ID NO:9. In some embodiments, a CT177 polypeptide antigen is a full-length CT177 polypeptide (e.g., the antigen comprises the amino acid sequence of SEQ ID NO:9). In some embodiments, a CT177 polypeptide antigen lacks one or more trans-membrane domains and/or a signal sequence (e.g., a CT177 polypeptide antigen lacks amino acids 1-30 of SEQ ID NO:9).

CT725 polypeptides are biotin synthetases. Exemplary amino acid and nucleotide sequences from a full-length CT725 polypeptide of C. trachomatis are shown below as SEQ IDs NO:11 and 12. In some embodiments, a CT725 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of a CT725 polypeptide sequence, e.g. at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of the sequence shown in SEQ ID NO:11 or of a sequence at least 60% (e.g., at least 65%, 70%, 75%, 80%, 85%, 90%, 95%, or 98%) identical to SEQ ID NO:11. In some embodiments, a CT725 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 105, 110, 120, 130, 140, 150, 160, 170, or 180 consecutive amino acids of the sequence shown in SEQ ID NO:11. In some embodiments, a CT725 polypeptide antigen is a full-length CT725 polypeptide (e.g., the antigen comprises the amino acid sequence of SEQ ID NO:11). In some embodiments, a CT726 polypeptide antigen lacks one or more trans-membrane domains (e.g., a CT726 polypeptide antigen lacks amino acids 51-75 and/or 116-136 of SEQ ID NO:11).

CT067 polypeptides are ABC transporter proteins. Exemplary amino acid and nucleotide sequences from a full-length CT067 polypeptide of C. trachomatis are shown below as SEQ IDs NO:23 and 24. In some embodiments, a CT067 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of a CT067 polypeptide sequence, e.g. at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of the sequence shown in SEQ ID NO:23 or of a sequence at least 60% (e.g., at least 65%, 70%, 75%, 80%, 85%, 90%, 95%, or 98%) identical to SEQ ID NO:23. In some embodiments, a CT067 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 325 consecutive amino acids of the sequence shown in SEQ ID NO:23. In some embodiments, a CT067 polypeptide antigen is a full-length CT067 polypeptide (e.g., the antigen comprises the amino acid sequence of SEQ ID NO:23). In some embodiments, a CT067 polypeptide antigen lacks one or more trans-membrane domains and/or a signal sequence (e.g., a CT067 polypeptide antigen lacks amino acids 1-33 and/or amino acids 11-31 of SEQ ID NO:23).

CT476 polypeptides are of unknown function. Exemplary amino acid and nucleotide sequences from a full-length CT476 polypeptide of C. trachomatis are shown below as SEQ IDs NO:63 and 64. In some embodiments, a CT476 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of a CT476 polypeptide sequence, e.g. at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of the sequence shown in SEQ ID NO:63 or of a sequence at least 60% (e.g., at least 65%, 70%, 75%, 80%, 85%, 90%, 95%, or 98%) identical to SEQ ID NO:63. In some embodiments, a CT476 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, or 320 consecutive amino acids of the sequence shown in SEQ ID NO:63. In some embodiments, a CT476 polypeptide antigen is a full-length CT476 polypeptide (e.g., the antigen comprises the amino acid sequence of SEQ ID NO:63). In some embodiments, a CT476 polypeptide antigen lacks one or more trans-membrane domains and/or a signal sequence (e.g., a CT476 polypeptide antigen lacks amino acids 1-18 and/or amino acids 1-20 of SEQ ID NO:63).

Chlamydia p6 polypeptides are plasmid virulence factors PGP4-D. Exemplary amino acid and nucleotide sequences from a full-length p6 polypeptide of C. trachomatis are shown below as SEQ IDs NO:65 and 66. In some embodiments, a p6 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of a p6 polypeptide sequence, e.g. at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of the sequence shown in SEQ ID NO:65 or of a sequence at least 60% (e.g., at least 65%, 70%, 75%, 80%, 85%, 90%, 95%, or 98%) identical to SEQ ID NO:65. In some embodiments, a p6 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, or 100 consecutive amino acids of the sequence shown in SEQ ID NO:65. In some embodiments, a p6 polypeptide antigen is a full-length p6 polypeptide (e.g., the antigen comprises the amino acid sequence of SEQ ID NO:65). In some embodiments, a p6 polypeptide antigen lacks one or more trans-membrane domains (e.g., a p6 polypeptide antigen lacks amino acids 52-68 of SEQ ID NO:65).

CT310 polypeptides are putative ATP synthase subunits. Exemplary amino acid and nucleotide sequences from a full-length CT310 polypeptide of C. trachomatis are shown below as SEQ IDs NO:67 and 68. In some embodiments, a CT310 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 160, 170, 180, 190, or 200 consecutive amino acids of a CT310 polypeptide sequence, e.g. at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 160, 170, 180, 190, or 200 consecutive amino acids of the sequence shown in SEQ ID NO:67 or of a sequence at least 60% (e.g., at least 65%, 70%, 75%, 80%, 85%, 90%, 95%, or 98%) identical to SEQ ID NO:67. In some embodiments, a CT310 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 160, 170, 180, 190, or 200 consecutive amino acids of the sequence shown in SEQ ID NO:67. In some embodiments, a CT310 polypeptide antigen is a full-length CT310 polypeptide (e.g., the antigen comprises the amino acid sequence of SEQ ID NO:67). In some embodiments, a CT310 polypeptide antigen lacks one or more trans-membrane domains (e.g., a CT310 polypeptide antigen lacks amino acids 117-136 of SEQ ID NO:67).

CT638 polypeptides are of unknown function. Exemplary amino acid and nucleotide sequences from a full-length CT638 polypeptide of C. trachomatis are shown below as SEQ IDs NO:69 and 70. In some embodiments, a CT638 polypeptide antigen includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, or 250 consecutive amino acids of a CT638 polypeptide sequence, e.g. at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, or 250 consecutive amino acids of the sequence shown in SEQ ID NO:69 or of a sequence at least 60% (e.g., at least 65%, 70%, 75%, 80%, 85%, 90%, 95%, or 98%) identical to SEQ ID NO:69. In some embodiments, a CT638 polypeptide antigen comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, or 250 consecutive amino acids of the sequence shown in SEQ ID NO:69. In some embodiments, a CT638 polypeptide antigen is a full-length CT310 polypeptide (e.g., the antigen comprises the amino acid sequence of SEQ ID NO:69). In some embodiments, a CT638 polypeptide antigen lacks one or more trans-membrane domains and/or a signal sequence (e.g., a CT638 polypeptide antigen lacks amino acids 1-33 and/or amino acids 13-31 of SEQ ID NO:69).

Exemplary amino acid and nucleotide sequences from full-length CT856, CT757, CT564, CT703, P1-ORF7, CT067, CT037, CT252, CT064, CT137, CT204, CT634, CT635, CT366, CT140, CT142, CT242, CT843, CT328, CT188, CT578, CT724, CT722, CT732, and CT788 polypeptide antigens are shown below as SEQ IDs NO:13-62. Exemplary amino acid and nucleotide sequences from full-length CT172, CT443, CT525, CT606, CT648, and CT870 polypeptide antigen are shown below as SEQ IDs NO:71-82.

Polypeptide antigens of Table 1 can be provided in any combination with each other and/or with other chlamydia antigens. In some embodiments, a combination of chlamydia polypeptide antigens includes two polypeptide antigens selected from Table 1. In some embodiments, a combination includes three polypeptide antigens selected from Table 1. In some embodiments, a combination includes four polypeptide antigens selected from Table 1. In some embodiments, a combination includes five polypeptide antigens selected from Table 1. In some embodiments, a combination includes six polypeptide antigens selected from Table 1. In some embodiments, a combination includes seven polypeptide antigens selected from Table 1. In some embodiments, a combination includes eight polypeptide antigens selected from Table 1.

Other antigens which can be provided in combination with one or more polypeptide antigens selected from Table 1, include one or more polypeptide antigens selected from Table 2, and/or one or more polypeptide antigens selected from Table 3. In some embodiments, a combination of antigens includes one, two, three, four, five, six, seven, or eight polypeptide antigens selected from Table 1, and one, two, three, four, five, or six polypeptide antigens selected from Table 2. In some embodiments, a combination of antigens includes one, two, three, four, five, six, seven, or eight polypeptide antigens selected from Table 1, and one, two, three, four, five, or six polypeptide antigens selected from Table 3. In some embodiments, a combination of antigens includes one, two, three, four, five, six, seven, or eight polypeptide antigens selected from Table 1; one, two, three, four, five, or six polypeptide antigens selected from Table 2; and one, two, three, four, five, or six polypeptide antigens selected from Table 3. In some embodiments, a combination of antigens includes one, two, three, four, five, or six polypeptide antigens selected from Table 2, and one, two, three, four, five, or six polypeptide antigens selected from Table 3. Antigens CT062, CT843, CT242, CT732, CT788, and specific epitopes of these antigens are described in PCT/US2007/004675 (published as WO 2007/098255), PCT/US2008/009282 (published as WO 2009/020553), PCT/US2008/013298 (published as WO 2009/073179), and PCT/US2009/068457 (published as WO 20010/078027), the entire contents of which are hereby incorporated by reference. Additional chlamydia polypeptide antigens that can be provided in combination with a novel antigen described herein include a polymorphic membrane protein D (PmpD or CT812; see GenBank NP_{—}220332.1 GI:15605546), a major outer membrane protein (MOMP or ompA or CT681; see GenBank NP_{—}220200.1 GI:15605414), CT858 or cpaf (GenBank NP_{—}220380 GI:15605594), CT713 or PorB (GenBank NP_{—}220232.1 GI:15605446), OMP85 (GenBank NP_{—}219746.1 GI:15604962), CT315 or RpoB (GenBank NP_{—}219820.1 GI:15605036), pgp3 or pORF 5 (GenBank NP 040384.1 GI:3205528), CT316, CT737, or CT674. Sequences of the above-mentioned polypeptides, and nucleic acids that encode them, are known. See, e.g., a C. trachomatis genome sequence in GenBank under Acc. No. NC_{—}000117, GI:15604717, annotated genes, and linked polypeptide sequences therein.

The present invention also provides compositions that include a chlamydia antigen described herein and an antigen from a different infectious agent. In some embodiments, a composition includes a chlamydia antigen and an antigen from a different infectious agent that causes a sexually transmitted disease. In some embodiments, compositions that include a chlamydia antigen (e.g., a polypeptide antigen selected from Table 1, Table 2, Table 3, or a combination thereof) and a papillomavirus antigen (e.g., a human papillomavirus antigen) are provided. In some embodiments, compositions that include a chlamydia antigen (e.g., a polypeptide antigen selected from Table 1, Table 2, Table 3, or a combination thereof) and a herpesvirus antigen (e.g., a human herpes simplex virus-2 antigen) are provided. In some embodiments, compositions that include a chlamydia antigen (e.g., a polypeptide antigen selected from Table 1, Table 2, Table 3, or a combination thereof) and a Neissiria gonorrhoea antigen are provided. In some embodiments, compositions that include a chlamydia antigen (e.g., a polypeptide antigen selected from Table 1, Table 2, Table 3, or a combination thereof) and a Candida albicans antigen are provided. In some embodiments, compositions that include a chlamydia antigen (e.g., a polypeptide antigen selected from Table 1, Table 2, Table 3, or a combination thereof) and an antigen from one or more of a papillomavirus, a herpesvirus (e.g., HSV-2), Neissiria gonorrhoeae, and Candida albicans are provided.

### Adjuvants

A large variety of formulations of immunogenic compositions can be employed to induce immune responses. A common route of administration in humans is by intramuscular (i.m.) injection, but immunogenic compositions may also be applied orally, intranasally, subcutaneously, by inhalation, intravenously, or by other routes of administration. In most cases, chlamydia antigens are initially presented to naive lymphocytes in regional lymph nodes.

In some embodiments, a chlamydia antigen composition includes purified components (e.g., purified antigens). In some embodiments, chlamydia antigens are fused to other molecules, such as proteins that can confer adjuvant activity, or moieties that facilitate isolation and purification (e.g., an epitope tag).

In some embodiments, a chlamydia antigen composition includes an adjuvant. In some embodiments, the adjuvant includes mineral-containing adjuvant. Mineral-containing ajduvants can be formulated as gels, in crystalline form, in amorphous form, as particles, etc. Mineral-containing adjuvants include, for example, aluminum salts and/or calcium salts (e.g., aluminum hydroxide, aluminum phosphate, aluminum sulfate, calcium phosphate, etc.). In some embodiments, a chlamydia antigen composition includes aluminum hydroxide. Alhydrogel™ is an example of an aluminum hydroxide gel adjuvant.

In some embodiments, an adjuvant includes an immunomodulatory oligonucleotide. In some embodiments, an immunomodulatory oligonucleotide sequence includes CpG (unmethylated cytosine-guanosine) motifs. Oligonucleotides having CpG motifs can include nucleotide analogs and/or non-naturally occurring internucleoside linkages (e.g., phosphorothioate linkages). For examples of various oligonucleotides include CpG motifs, see Kandimalla, et al., Nuc. Acids Res. 31(9): 2393-2400, 2003; WO02/26757; WO99/62923; Krieg, Nat. Med. 9(7): 831-835, 2003; McCluskie, et al., FEMS Immunol. Med. Microbiol. 32:179-185, 2002; WO98/40100; U.S. Pat. No. 6,207,646; U.S. Pat. No. 6,239,116 and U.S. Pat. No. 6,429,199. Other immunomodulatory nucleotide sequences double stranded RNA sequences, palindromic sequences, and poly(dG) sequences.

In some embodiments, an adjuvant comprises IC₃₁™ (Intercell AG). IC31™ is a synthetic adjuvant that includes an antimicrobial peptide, KLK, and an immunostimulatory oligonucleotide, ODN1a, and acts as a Toll-like Receptor 9 (TLR9) agonist.

In some embodiments, an adjuvant includes a toxin. In some embodiments, a toxin is a bacterial ADP-ribosylating toxin, e.g., cholera toxin, E. coli heat labile toxin, or pertussis toxin. In some embodiments, the bacterial toxin is a detoxified form of an ADP-ribosylating toxin (see, e.g., Beignon, et al., Inf. Immun. 70(6):3012-3019, 2002; Pizza, et al., Vaccine 19:2534-2541, 2001; Pizza, et al., Int. J. Med. Microbiol. 290(4-5):455-461, 2000; Scharton-Kersten et al., Inf. Immun.68(9):5306-5313, 2000; Ryan et al., Inf. Immun 67(12):6270-6280, 1999; Partidos et al., Immunol. Lett. 67(3):209-216, 1999; Peppoloni et al., Vaccines 2(2):285-293, 2003; and Pine et al., J. Control Release 85(1-3):263-270, 2002).

In some embodiments, an adjuvant includes an endotoxin such as monophosphoryl lipid A or 3-De-O-acylated monophosphoryl lipid A (see U.S. Pat. No. 4,987,237 and GB 2122204B).

In some embodiments, an adjuvant includes a muramyl dipeptide (e.g., N-acetyl-muramyl-L-threonyl-D-isoglutamine(thr-MDP), N-acetyl-normuramyl-1-alanyl-d-isoglutamine(nor-MDP), and N-acetylmuramyl-1-alanyl-d-isoglutaminyl-1-alanine-2-(1′-2′-dipalmitoyl-s-n-glycero-3-hydroxyphosphoryloxy)-ethylamine MTP-PE).

In some, an adjuvant includes an oil emulsion and/or emulsifier-based adjuvant. In some embodiments, an oil emulsion adjuvant includes a Freund's Adjuvant (e.g., Complete Freund's adjuvant (CFA), or incomplete Freund's adjuvant (IFA)). In some embodiments, an oil-emulsion adjuvant includes a squalene water emulsion, such as MF59 (Novartis; see, e.g., WO9014837), or a Synex adjuvant formulation (SAF)). In some embodiments, an oil emulsion includes a dispersing agent, e.g., a mono- or di-C₁₂-C₂₄-fatty acid ester of sorbitan or mannide, e.g., sorbitan mono-stearate, sorbitan mon-oleate, or mannide mono-oleate. Examples of oil emulsions that include squalene and dispersing agents includes Arlacel™, Montanide™ ISA-720, and Montanide™ ISA-703. Other oil emulsions are described, e.g., in WO 95/17210 and EP 0399842.

In some embodiments, an adjuvant includes a saponin. Saponins are steroid and/or triterpenoid glycosides derived from plants such as Quillaja saponaria, Saponaria officianalis, Smilax ornata, and Gypsophilla paniculata. Fractions of saponin-containing extracts that have been described and that can be used as adjuvants for chlamydia antigens include Quil™A, QS21, QS7, QS17, QS18, QH-A, QH-B, QH-C, and QuilA (see, e.g., U.S. Pat. No. 5,057,540). In some embodiments, QS21 is used as an adjuvant.

In some embodiments, an adjuvant includes an immune stimulating complex (ISCOM). ISCOMs are particles that typically include a glycoside (e.g., a saponin) and a lipid. In some embodiments, an ISCOM includes a saponin and a cholesterol. In some embodiments, an ISCOM includes a saponin, a cholesterol, and a phospholipid (e.g., phosphatidylcholine and/or phosphatidylethanolamine). In some embodiments, an ISCOM includes a nonionic block copolymer. ISCOMs can include additional adjuvants, e.g., additional adjuvant substances described herein (see, e.g., WO 05/002620). In some embodiments, an ISCOM includes a substance that targets it to a mucosal membrane (see, e.g., WO97/030728). Other ISCOM compositions and preparation of the compositions suitable for combination with chlamydia antigens provided herein are described, e.g., in U.S. Pat. Pub. No. 20060121065, WO 00/07621, WO 04/004762, WO 02/26255, and WO 06/078213. In some embodiments, an adjuvant comprises an AbISCO® adjuvant (e.g., Matrix-M™, Isconova). In some embodiments, an adjuvant comprises AbISCO®-100. In some embodiments, an adjuvant comprises AbISCO®-300.

In some embodiments, an adjuvant includes a nonionic block copolymer. Nonionic block copolymers typicaly include two chains of hydrophobic polyoxyethylenes of various lengths combined with a block of hydrophobic polyoxypropylene. In some embodiments, a nonionic block copolymer is formulated in an oil-in-water emulsion (e.g., with oil and squalene).

In some embodiments, an adjuvant includes virus like particles (VLPs). VLPs are non replicating, non infectious particles that typically include one or more viral proteins, optionally formulated with an additional component such as a phospholipid. In some embodiments, a VLP includes proteins from one or more of the following: an influenza virus (e.g., a hemaglutinin (HA) or neuraminidase (NA) polyptide), Hepatitis B virus (e.g., a core or capsid polypeptide), Hepatitis E virus, measles virus, Sindbis virus, Rotavirus, Foot-and-Mouth Disease virus, Retrovirus, Norwalk virus, human papilloma virus, HIV, RNA-phages, Q13-phage (e.g., a coat protein), GA-phage, fr-phage, AP205 phage, a Ty (e.g., retrotransposon Ty protein p1). See, e.g., WO03/024480, WO03/024481, WO08/061,243, and WO07/098,186.

In some embodiments, an adjuvant includes replicons. Replicons resemble VLPs in that they are noninfectious particles including viral proteins, and further include a nucleic acid encoding a polypeptide (e.g., an antigen). In some embodiments, a replicon includes proteins from an alphavirus. Alphaviruses include, e.g., Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J Virus, Fort Morgan Virus, Ndumu Virus, and Buggy Creek Virus. In some embodiments, an adjuvant includes a replicon that includes a nucleic acid encoding one or more chlamydia antigens described herein. In some embodiments, an adjuvant includes a replicon that encodes a cytokine (e.g., interleukin-12 (IL-12), IL-23, or granulocyte-macrophage colony-stimulating factor (GM-CSF)). Production and uses of replicons are described, e.g., in WO08/058,035, WO08/085,557, and WO08/033,966). In some embodiments, a VLP or replicon adjuvant includes one or more chlamydia antigens (i.e., VLP or replicon particles include a chlamydia antigen as part of the particles). In some embodiments, a VLP or replicon adjuvant is co-adminstered with a chlamydia antigen polypeptide.

In some embodiments, an adjuvant includes liposomes, which are are artificially-constructed spherical lipid vesicles (see, e.g., U.S. Pat. Nos. 4,053,585; 6,090,406; and 5,916,588). In certain embodiments, a lipid to be used in liposomes can be, but is not limited to, one or a plurality of the following: phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and lyso-phosphatides. In some embodiments, an adjuvant includes a liposome and a ligand for a Toll-like Receptor (TLR; see, e.g., WO/2005/013891, WO/2005/079511, WO/2005/079506, and WO/2005/013891). In some embodiments, an adjuvant includes JVRS-100. JVRS-100 comprises cationic liposomes combined with non-coding oligonucleotides or plasmids.

In some embodiments, an adjuvant includes microparticles comprised of a polymer, e.g., a polymer of acrylic or methacrylic acid, polyphosphazenes, polycarbonates, polylactic acid, polyglycolic acid, copolymers of lactic acid or glycolic acid, polyhydroxybutyric acid, polyorthoesters, polyanhydrides, polysiloxanes, polycaprolactone, or a copolymer prepared from the monomers of these polymers. In some embodiments, an adjuvant includes microparticles comprised of a polymer selected from the group consisting of polyvinylpyrrolidone, polyvinylalcohol, polyhydroxyethylmethacrylate, polyacrylamide, polymethacrylamide, and polyethyleneglycol (see, e.g., U.S. Pat. No. 5,500,161).

In some embodiments, an adjuvant includes biodegradable microspheres (e.g., microspheres comprised of poly(D,L-lactic acid), poly(D,L-glycolic acid), poly(ε-caprolactone), polye (α-hydroxy actid), polyhydroxybutyric acid, a polyorthoester, a polyanhydride, etc.).

In some embodiments, an adjuvant includes a cytokine. In some embodiments, an adjuvant includes IL-12. In some embodiments, an adjuvant includes IL-23. In some embodiments, an adjuvant includes GM-CSF.

In some embodiments, an adjuvant includes a lipopeptide. In some embodiments, an adjuvant includes a Pam-3-Cys lipopeptide. In some embodiments, an adjuvant including a lipopeptide activates Toll-like receptors (TLRs).

### Modifications

The chlamydia antigens described herein may be used with or without modification. In some embodiments, a chlamydia antigen may be modified to elicit the desired immune response. In some embodiments, a chlamydia antigen is conjugated to an appropriate immunogenic carrier such as tetatus toxin, pneumolysin, keyhole limpet hemocyanin, or the like. In some embodiments, a chlamydia polypeptide antigen is post-translationally modified, e.g. by phosphorylation, myristoylation, acylation, glycosylation, glycation, and the like. In some embodiments, a chlamydia polypeptide antigen is lipidated. Conjugation to the lipid moiety may be direct or indirect (e.g., via a linker). The lipid moiety may be synthetic or naturally produced. In some embodiments, a chlamydia polypeptide antigen is chemically conjugated to a lipid moiety. In some embodiments, a DNA construct encoding a chlamydia polypeptide antigen comprises a lipidation sequence. A lipidation sequence may be N-terminal or C-terminal to the polypeptide, and may be embedded in a signal or other sequence. An exemplary lipidation sequence is the signal sequence of the E. coli gene RlpB, shown as SEQ ID NO:83.

In some embodiments, a chlamydia polypeptide antigen is covalently bound to another molecule. This may, for example, increase the half-life, solubility, bioavailability, or immunogenicity of the antigen. Molecules that may be covalently bound to the antigen include a carbohydrate, biotin, poly(ethylene glycol) (PEG), polysialic acid, N-propionylated polysialic acid, nucleic acids, polysaccharides, and PLGA. In some embodiments, the naturally produced form of a polypeptide is covalently bound to a moiety that stimulates the immune system. An example of such a moiety is a lipid moiety. In some instances, lipid moieties are recognized by a Toll-like receptor (TLR) such as TLR2 or TLR4 and activate the innate immune system.

### Nucleic Acid Compositions and Antigen Expression

Various types of vectors are suitable for expression of chlamydia antigens in an expression system (e.g., in a host cell). In some embodiments, a composition includes a vector suitable for expression in vitro (whether in a cell or in a cell-free system), e.g., for producing a polypeptide composition. The term “vector” refers to a nucleic acid molecule capable of transporting another nucleic acid to which it has been linked and can include, for example, a plasmid, cosmid or viral vector. The vector can be capable of autonomous replication or it can integrate into a host DNA. Viral vectors include, e.g., replication defective retroviruses, adenoviruses and adeno-associated viruses. Other types of viral vectors are known in the art.

A vector can include a nucleic acid encoding a chlamydia antigen in a form suitable for expression of the nucleic acid in a host cell. A recombinant expression vector typically includes one or more regulatory sequences operatively linked to the nucleic acid sequence to be expressed. Regulatory sequences include promoters, enhancers and other expression control elements (e.g., polyadenylation signals). Regulatory sequences include those which direct constitutive expression of a nucleotide sequence, as well as tissue-specific regulatory and/or inducible sequences. A sequence encoding a chlamydia antigen can include a sequence encoding a signal peptide (e.g., a heterologous signal peptide) such that the antigen is secreted from a host cell. The design of the expression vector can depend on such factors as the choice of the host cell to be transformed, the level of expression of protein desired, and the like.

Recombinant expression vectors can be designed for expression and production of chlamydia antigens in prokaryotic or eukaryotic cells. For example, antigens can be expressed in E. coli, insect cells (e.g., using baculovirus expression vectors), yeast cells or mammalian cells. Suitable host cells are discussed further in Goeddel, Gene Expression Technology Methods in Enzymology 185, Academic Press, San Diego, Calif., 1990. Alternatively, a recombinant expression vector can be transcribed and translated in vitro, for example using T7 promoter regulatory sequences and T7 polymerase.

Expression of polypeptides in prokaryotes is often carried out in E. coli with vectors containing constitutive or inducible promoters directing the expression of either fusion or non-fusion proteins. Fusion vectors add a number of amino acids to a protein encoded therein, e.g., to the amino terminus or carboxy terminus of the recombinant protein, e.g., to increase expression of recombinant protein; to increase the solubility of the recombinant protein; and/or to aid in the purification of the recombinant antigen by acting as a ligand in affinity purification. Often, a proteolytic cleavage site is introduced at the junction of the fusion moiety and the recombinant antigen to enable separation of the recombinant antigen from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. Gene 67:31-40, 1988), pMAL (New England Biolabs, Beverly, Mass.) and pRITS (Pharmacia, Piscataway, N.J.) which fuse glutathione S-transferase (GST), maltose E binding protein, or protein A, respectively, to the target recombinant protein. Chlamydia antigen expression vectors provided herein include yeast expression vectors, vectors for expression in insect cells (e.g., a baculovirus expression vector) and vectors suitable for expression in mammalian cells.

An expression vector for use in mammalian cells can include viral regulatory elements. For example, commonly used promoters are derived from polyoma, Adenovirus 2, cytomegalovirus and Simian Virus 40. A vector can include an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard, Proc. Natl. Acad. Sci. USA 89:5547, 1992, and Paillard, Human Gene Therapy 9:983, 1989).

A host cell can be any prokaryotic or eukaryotic cell. For example, a chlamydia antigen can be expressed in bacterial cells (such as E. coli), insect cells, yeast or mammalian cells (such as Chinese hamster ovary cells (CHO) or COS cells (African green monkey kidney cells CV-1 origin SV40 cells; Gluzman, Cell 23:175-182, 1981). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, gene gun, or electroporation.

A host cell can be used to produce (i.e., express) a chlamydia antigen. Accordingly, the invention further provides methods for producing a chlamydia antigen using host cells. In one embodiment, the method includes culturing a host cell (into which a recombinant expression vector encoding a chlamydia antigen has been introduced) in a suitable medium such that a chlamydia antigen is produced. In another embodiment, the method further includes isolating a chlamydia antigen from the medium or the host cell. Purified chlamydia antigens can be used for administration to mammals to induce an immune response, and/or to generate antibodies specific for the antigens.

The present invention also provides nucleic acid compositions that encode chlamydia antigens for administration to a subject in vivo, e.g., to elicit an immune response to the antigen. In some embodiments, a nucleic acid composition for administration in vivo includes a naked DNA plasmid encoding a chlamydia antigen. Bacterial vectors, replicon vectors, live attenuated bacteria, and viral vectors for expression of heterologous genes also can be used. Live attenuated viral vectors (e.g., recombinant vaccinia (e.g., modified vaccinia Ankara (MVA), IDT Germany), recombinant adenovirus, avian poxvirus (e.g., canarypox (e.g., ALVAC™, Aventis Pasteur) or fowlpox), poliovirus, and alphavirus virion vectors) have been successful in inducing cell-mediated immune response to antigens. Avian poxviruses are defective in mammalian hosts, but can express inserted heterologous genes under early promoters. Recombinant adenovirus and poliovirus vectors can thrive in the gut and so can stimulate efficient mucosal immune responses. Finally, attenuated bacteria can also be used as a vehicle for DNA vaccine delivery. Examples of suitable bacteria include S. enterica, S. tymphimurium, Listeria, and BCG. The use of mutant bacteria with weak cell walls can aid the exit of DNA plasmids from the bacterium.

Nucleic acid compositions used for immunization can include an adjuvant (e.g., an adjuvant such as a polymer, a saponin, muramyl dipeptide, liposomes, immunomodulatory oligonucleotide, or another adjuvant described herein) to promote nucleic acid uptake. Regardless of route, adjuvants can be administered before, during, or after administration of the nucleic acid. In some embodiments, an adjuvant increases the uptake of nucleic acid into host cells and/or increases expression of the antigen from the nucleic acid within the cell, induce antigen presenting cells to infiltrate the region of tissue where the antigen is being expressed, or increase the antigen-specific response provided by lymphocytes.

### Antibodies

This invention provides, inter alia, antibodies, or antigen-binding fragments thereof, to a novel chlamydia antigen described herein, e.g., a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, a CT476 polypeptide antigen, a p6 polypeptide antigen, a CT310 polypeptide antigen, or a CT638 polypeptide antigen. The antibodies can be of the various isotypes, including: IgG (e.g., IgG1, IgG2, IgG3, IgG4), IgM, IgA1, IgA2, IgD, or IgE. In some embodiments, an antibody is an IgG isotype, e.g., IgG1. An antibody against a chlamydia antigen can be full-length (e.g., an IgG1 or IgG4 antibody) or can include only an antigen-binding fragment (e.g., a Fab, F(ab)₂, Fv or a single chain Fv fragment). These include monoclonal antibodies, recombinant antibodies, chimeric antibodies, human antibodies, and humanized antibodies, as well as antigen-binding fragments of the foregoing.

Monoclonal antibodies can be produced by a variety of techniques, including conventional monoclonal antibody methodology, e.g., the standard somatic cell hybridization technique of Kohler and Milstein, Nature 256: 495, 1975. Polyclonal antibodies can be produced by immunization of animal or human subjects. See generally, Harlow, E. and Lane, D. Antibodies: A Laboratory Manual, Cold Spring Harbor Laboratory Press, Cold Spring Harbor, N.Y., 1988. Antibodies against chlamydia antigens described herein can be used, e.g., for diagnostic assays, or for therapeutic applications.

In some embodiments of the present invention, a subject's response to an immunogenic composition described herein is evaluated, e.g., to determine efficacy of the composition, and/or to compare responses elicited by the composition to responses elicited by a different composition.

### Assays for T Cell Activation

In some embodiments, various assays can be utilized in order to characterize an antigen or composition and/or to determine whether an immune response has been stimulated in a T cell or group of T cells. In some embodiments, assays are used to characterize a T cell response in a subject that has been administered an immunogenic composition to elicit an anti-chlamydia response (e.g., to evaluate whether a detectable T cell response has been elicited and/or to evaluate the potency of the response). The novel chlamydia antigens described herein also provide diagnostic agents to evaluate exposure to chlamydia infections (e.g., in non-vaccinated subjects). In some embodiments, assays are used to characterize a T cell response in a subject to determine whether the subject has been infected with a chlamydia organism. The subject can be a subject suspected of exposure to a chlamydia organism recently (i.e., an assay to detect a response can be performed with a sample taken from the subject about 3, 4, 5, 6, 7, 8, 9, 10, 14, 30, or more days after suspected exposure to a chlamydia organism). The subject can be a subject suspected of exposure to a chlamydia organism weeks, months, or years prior to the assay. The novel chlamydia antigens described herein also provide prognostic agents to evaluate outcomes of exposure to a chlamydia organism (e.g., in subjects known to be, or to have been, infected with a chlamydia organism). In some embodiments, assays are used to characterize a T cell response in a subject to assess the likelihood of sequelae (e.g., pelvic inflammatory disease and infertility) to infection with a chlamydia organism.

In some embodiments, stimulation of an immune response in T cells is determined by measuring antigen-induced production of cytokines by T cells. In some embodiments, stimulation of an immune response in T cells can be determined by measuring antigen-induced production of IFN-γ, IL-4, IL-2, IL-6, IL-10, IL-17 and/or TNF-α by T cells. In some embodiments, antigen-induced production of cytokines by T cells can be measured by intracellular cytokine staining followed by flow cytometry. Other suitable methods include surface capture staining followed by flow cytometry, or methods that determine cytokine concentration in supernatants of activated T cell cultures, such as ELISA or ELISPOT assays.

In some embodiments, antigen-produced production of cytokines by T cells is measured by ELISPOT assay. ELISPOT assays typically employ a technique very similar to the sandwich enzyme-linked immunosorbent assay (ELISA) technique. An antibody (e.g. monoclonal antibody, polyclonal antibody, etc.) is coated aseptically onto a PVDF (polyvinylidene fluoride)-backed microplate. Antibodies are chosen for their specificity for the cytokine of interest. The plate is blocked (e.g., with a serum protein that is non-reactive with any of the antibodies in the assay). Cells to be tested for cytokine production are plated out at varying densities, along with antigen or mitogen, and then placed in a humidified 37° C. CO₂ incubator for a specified period of time. Cytokine secreted by activated cells is captured locally by the coated antibody on the high surface area PVDF membrane. After washing the wells to remove cells, debris, and media components, a secondary antibody (e.g. a biotinylated polyclonal antibody) specific for the cytokine is added to the wells. This antibody is reactive with a distinct epitope of the target cytokine and thus is employed to detect the captured cytokine. Following a wash to remove any unbound biotinylated antibody, the detected cytokine is then visualized using an avidin-HRP, and a precipitating substrate (e.g., AEC, BCIP/NBT). The colored end product (a spot, usually red or blue) typically represents an individual cytokine-producing cell. Spots can be counted manually (e.g., with a dissecting microscope) or using an automated reader to capture the microwell images and to analyze spot number and size. In some embodiments, each spot correlates to a single cytokine-producing cell.

In some embodiments, an immune response in T cells is said to be stimulated if between about 1% and about 100% of antigen-specific T cells produce cytokines. In some embodiments, an immune response in T cells is said to be stimulated if at least about 1%, at least about 5%, at least about 10%, at least about 25%, at least about 50%, at least about 75%, at least about 90%, at least about 95%, at least about 99%, or about 100% of antigen-specific T cells produce cytokines.

In some embodiments, an immune response in T cells is said to be stimulated if immunized subjects comprise at least about 10-fold, at least about 50-fold, at least about 100-fold, at least about 500-fold, at least about 1000-fold, at least about 5000-fold, at least about 10.000-fold, at least about 50.000-fold, at least about 100.000-fold, or greater than at least about 100.000-fold more cytokine-producing cells than do naïve controls.

In some embodiments, stimulation of an immune response in T cells can be determined by measuring antigen-induced proliferation of T cells. In some embodiments, antigen-induced proliferation may be measured as uptake of H³-thymidine in dividing T cells (sometimes referred to as “lymphocyte transformation test, or “LTT”). In some embodiments, antigen-induced proliferation is said to have occurred if ³H-thymidine uptake (given as number of counts from a γ counter) is at least about 5-fold, at least about 10-fold, at least about 20-fold, at least about 50-fold, at least about 100-fold, at least about 500-fold, at least about 1000-fold, at least about 5000-fold, at least about 10.000-fold, or greater than at least about 10.000-fold higher than a naïve control.

In some embodiments, antigen-induced proliferation may be measured by flow cytometry. In some embodiments, antigen-induced proliferation may be measured by a carboxyfluorescein succinimidyl ester (CFSE) dilution assay. CFSE is a non-toxic, fluorescent, membrane-permeating dye that binds the amino groups of cytoplasmic proteins with its succinimidyl-reactive group (e.g., T cell proteins). When cells divide, CFSE-labeled proteins are equally distributed between the daughter cells, thus halving cell fluorescence with each division. Consequently, antigen-specific T cells lose their fluorescence after culture in the presence of the respective antigen (CFSE^{low}) and are distinguishable from other cells in culture (CFSE^{high}). In some embodiments, antigen-induced proliferation is said to have occurred if CFSE dilution (given as the percentage of CFSE^{low} cells out of all CFSE⁺ cells) is at least about 5%, at least about 10%, at least about 25%, at least about 50%, at least about 75%, at least about 90%, at least about 95%, or at least about 100%.

In some embodiments, an immune response in T-cells is said to be stimulated if cellular markers of T cell activation are expressed at different levels (e.g., higher or lower levels) relative to unstimulated cells. In some embodiments, CD11a, CD27, CD25, CD40L, CD44, CD45RO, and/or CD69 are more highly expressed in activated T cells than in unstimulated T cells. In some embodiments, L-selectin (CD62L), CD45RA, and/or CCR7 are less highly expressed in activated T cells than in unstimulated T cells.

In some embodiments, an immune response in T cells is measured by assaying cytotoxicity by effector CD8⁺ T cells against antigen-pulsed target cells. For example, a ⁵¹chromium (⁵¹Cr) release assay can be performed. In this assay, effector CD8⁺ T cells bind infected cells presenting virus peptide on class I MHC and signal the infected cells to undergo apoptosis. If the cells are labeled with ⁵¹Cr before the effector CD8⁺ T cells are added, the amount of ⁵¹Cr released into the supernatant is proportional to the number of targets killed. In some embodiments, an immune response in T cells is measured by an in vivo cytotoxicity assay in which target cells are antigen pulsed and labeled with a fluorescent dye, then transferred into immunized animals. Specific cytolytic T cells cause the disappearance of fluorescently labeled cells that are pulsed with a relevant antigen, but no decrease in cells pulsed with a control antigen. See, e.g., Coligan et al., Current Protocols in Immunology, 3.11.14-16, John Wiley & Sons, Inc., 2007. In some embodiments, an immune response in T cells is measured by detecting expression of one or more of Perforin, Granzyme B, or CD107a (e.g., by ELISPOT or flow cytometry). See, e.g., Betts et al., J. Immunol. Meth. 281(1-2):65-78, 2003.

### Assays for B Cell Activation

In some embodiments, various assays can be utilized in order to determine whether an immune response has been stimulated in a B cell or group of B cells, e.g., to characterize an antibody response in a subject that has been administered an immunogenic composition against chlamydia, or to determine whether a subject has been exposed to a chlamydia organism. In some embodiments, stimulation of an immune response in B cells can be determined by measuring antibody titers. In general, “antibody titer” refers to the ability of antibodies to bind antigens at particular dilutions. For example, a high antibody titer refers to the ability of antibodies to bind antigens even at high dilutions. In some embodiments, an immune response in B cells is said to be stimulated if antibody titers are measured to be positive at dilutions at least about 5-fold greater, at least about 10-fold greater, at least about 20-fold greater, at least about 50-fold greater, at least about 100-fold greater, at least about 500-fold greater, at least about 1000 fold greater, or more than about 1000-fold greater than in non-immunized individuals or pre-immune serum.

In some embodiments, stimulation of an immune response in B cells can be determined by measuring antibody affinity. In particular, an immune response in B cells is said to be stimulated if an antibody that has an equilibrium dissociation constant (Kₐ) less than 10⁻⁷ M, less than 10⁻⁸ M, less than 10⁻⁹ M, less than 10⁻¹⁰ M, less than 10⁻¹¹ M, less than 10⁻¹² M, or less, has been elicited.

In some embodiments, a T cell-dependent immune response in B cells is said to be stimulated if class-switch recombination has occurred. In particular, a switch from IgM to another isotype (e.g., to an IgG isotype or to IgA or to a mixture of these isotypes) is indicative of a T-cell dependent immune response in B cells.

In some embodiments, an immune response in B cells is determined by measuring affinity maturation of antigen-specific antibodies. Affinity maturation occurs during the germinal center reaction whereby activated B cells repeatedly mutate a region of the immunoglobulin gene that encodes the antigen-binding region. B cells producing mutated antibodies which have a higher affinity for antigen are preferentially allowed to survive and proliferate. Thus, over time, the antibodies made by B cells in GCs acquire incrementally higher affinities. In some embodiments, the readout of this process is the presence of high antibody titer (e.g. high affinity IgG antibodies that bind and neutralize antigens even at high dilutions).

In some embodiments, an immune response in B cells is said to be stimulated if memory B cells and/or long-lived plasma cells that can produce large amounts of high-affinity antibodies for extended periods of time have formed. In some embodiments, antibody titers are measured after different time intervals (e.g. 2 weeks, 1 month, 2 months, 6 months, 1 year, 2 years, 5 years, 10 years, 15 years, 20 years, 25 years, or longer) after vaccination in order to test for the presence of memory B cells and/or long-lived plasma cells that can produce large amounts of high-affinity antibodies for extended periods of time. In some embodiments, memory B cells and/or long-lived plasma cells that can produce large amounts of high-affinity antibodies for extended periods of time are said to be present by measuring humoral responses (e.g. if humoral responses are markedly more rapid and result in higher titers after a later booster vaccination than during the initial sensitization).

In some embodiments, an immune response in B cells is said to be stimulated if a vigorous germinal center reaction occurs. In some embodiments, a vigorous germinal center reaction can be assessed visually by performing histology experiments. In some embodiments, vigorous germinal center reaction can be assayed by performing immunohistochemistry of antigen-containing lymphoid tissues (e.g., vaccine-draining lymph nodes, spleen, etc.). In some embodiments, immunohistochemistry is followed by flow cytometry.

In some embodiments, stimulation of an immune response in B cells can be determined by identifying antibody isotypes (e.g., IgG, IgA, IgE, IgM). In certain embodiments, production of IgG isotype antibodies by B cells is a desirable immune response by B cells. In certain embodiments, production of IgA isotype antibodies by B cells is a desirable immune response by B cells.

In some embodiments, an immune response in B cells is determined by analyzing antibody function in neutralization assays. In one example, the ability of a chlamydia organism to infect a susceptible cell in vitro in the absence of serum is compared to conditions when different dilutions of immune and non-immune serum are added to the culture medium in which the cells are grown. In certain embodiments, an immune response in a B cell is said to be stimulated if infection by a chlamydia organism is neutralized at a dilution of about 1:5, about 1:10, about 1:50, about 1:100, about 1:500, about 1:1000, about 1:5000, about 1:10,000, or less. Assays for neutralization of chlamydia are described, e.g., in Peeling et al., Infect. Immun. 46:484-488, 1984; and Peterson et al., Infect. Immun. 59:4147-4153, 1991.

### In Vivo Assays

In some embodiments, an immunogenic composition may be characterized (e.g., to assess efficacy in inducing a beneficial response in animal models) by infecting groups of immunized and non-immunized mice (e.g., 3 or more weeks after vaccination) with a dose of a chlamydia organism that typically produces a particular pathology (e.g., upper urogenital tract infection) or bacterial burden. The magnitude and duration of pathology or bacterial burden due to infection of both groups is monitored and compared. In one example, B cell responses are characterized by transferring serum from immune mice as a “passive vaccine” to assess protection of non-immune mice from pathological effects or burden of infection. In some embodiments, infiltrating leukocyte populations are characterized (e.g., to assess the number and type cells in a region of infection, e.g., whether CD4⁺ T cells, CD8⁺ T cells, or other cell types are present). Animal models for chlamydial urogenital infection have been described. In some embodiments, a chlamydia organism is applied as an intravaginal inoculum, and infection and pathology of one or more of lower and upper genital tracts of the infected animal is characterized. See, e.g., Barron et al. (J. Infect. Dis. 143(1):63-6, 1981), which describes an intravaginal infection model in mice. In some embodiments, clearance of primary infection is a measure of protective immunity in this model. In some embodiments, detection of CD4⁺ T cell responses of a Th1 subtype correlate with protection (Morrison et al., Infect. Immun 70:2741-2751, 2002).

In some embodiments, an immunogenic composition is assessed in an animal model of chlamydia infection. In some embodiments, lower urogenital tract infection by chlamydia is assessed in the model (e.g., lower tract bacterial burden and/or inflammation due to infection is assessed). In some embodiments, upper tract infection by chlamydia is assessed in the model (e.g., one or more of upper tract bacterial burden, inflammation, infertility, collagen deposition, scarring due to infection, are assessed). In some embodiments, an ability to prevent ascension of a chlamydia infection from the lower tract to the upper genital tract is assessed. In some embodiments, rate of bacterial clearance from the lower tract is assessed. In some embodiments, rate of bacterial clearance from the upper tract is assessed. In some embodiments, an immunogenic composition is assessed in an animal model in multiple strains of the animal of interest (e.g., multiple mouse strains). In some embodiments, presence and size of hydrosalpinx (fluid blockage of fallopian tubes) is assessed.

In some embodiments, desirable immunogenic compositions are characterized as having one or more of the above effects in vivo (e.g., in an animal model). For example, in some embodiments, an immunogenic composition reduces lower urogenital tract infection by chlamydia bacteria. In some embodiments, an immunogenic composition reduces lower tract bacterial burden. In some embodiments, an immunogenic composition reduces lower tract inflammation due to infection. In some embodiments, an immunogenic composition reduces upper tract infection by chlamydia. In some embodiments, an immunogenic composition reduces one or more of upper tract bacterial burden, inflammation, infertility, collagen deposition, scarring due to a chlamydia infection. In some embodiments, an immunogenic composition reduces ascension of a chlamydia infection from the lower tract to the upper genital tract. In some embodiments, an immunogenic composition increases the rate of bacterial clearance from the lower tract and/or the upper tract. In some embodiments, an immunogenic composition reduces presence and/or size of hydrosalpinx or salpyngitis due to infection. In some embodiments, an immunogenic composition has one or more of the above effects in multiple animal strains (e.g., multiple mouse strains).

One of ordinary skill in the art will recognize that the assays described above are only exemplary methods which could be utilized in order to determine whether T cell activation and/or B cell activation has occurred. Any assay known to one of skill in the art which can be used to determine whether T and/or B cell activation has occurred falls within the scope of this invention. The assays described herein as well as additional assays that could be used to determine whether T and/or B cell activation has occurred are described in Current Protocols in Immunology (John Wiley & Sons, Hoboken, N.Y., 2007; incorporated herein by reference).

### Applications

The compositions and methods described herein can be used for the prophylaxis and/or treatment of any chlamydia infection, chlamydial disease, disorder, and/or condition. As used herein, “prophylaxis” refers to uses before onset of symptoms due to a chlamydia infection, chlamydial disease, disorder, and/or condition and/or before known exposure to a chlamydia organism. Subjects include, but are not limited to, humans and/or other primates; and other animals susceptible to infection by chlamydia organisms, including commercially relevant mammals such as cattle, pigs, horses, sheep, cats, and/or dogs; and/or birds, including commercially relevant birds such as chickens, ducks, geese, and/or turkeys.

In some embodiments, immunogenic compositions in accordance with the present invention may be used to treat, alleviate, ameliorate, relieve, delay onset of, inhibit progression of, reduce risk of infection by, and reduce severity of, and/or reduce incidence of one or more symptoms or features of a chlamydial disease, disorder, and/or condition. In some embodiments, inventive an immunogenic composition may be used to treat, alleviate, ameliorate, relieve, delay onset of, inhibit progression of, reduce severity of, and/or reduce incidence of one or more symptoms or features of chlamydial infection (e.g., C. trachomatis infection, C. pneumoniae infection, C. psittaci infection).

In one aspect of the invention, a method for the prophylaxis and/or treatment of chlamydia infection is provided. In some embodiments, the prophylaxis and/or treatment of chlamydia infection comprises administering a therapeutically effective amount of an immunogenic composition described herein to a subject in need thereof, in such amounts and for such time as is necessary to achieve the desired result. In certain embodiments of the present invention a “therapeutically effective amount” of an inventive immunogenic composition is that amount effective for reducing risk of infection by, or treating, alleviating, ameliorating, relieving, delaying onset of, inhibiting progression of, reducing severity of, and/or reducing incidence of one or more symptoms or features of chlamydia infection. A therapeutically effective amount may be determined on a population basis, and is not required to be an amount that naturally induces a protective response in a particular subject.

In some embodiments, inventive prophylactic and/or therapeutic protocols involve administering a therapeutically effective amount of one or more inventive immunogenic compositions to a healthy subject (i.e., a subject who does not display any symptoms of chlamydia infection and/or who has not been diagnosed with chlamydia infection). For example, healthy individuals may be vaccinated using inventive immunogenic compositions prior to development of chlamydia infection and/or onset of symptoms of chlamydia infection; at risk individuals (e.g., patients exposed to individuals suffering from chlamydia infection, patients at high risk for sexually transmitted diseases, individuals at risk due to young age (e.g., children, adolescents, or young adults)) can be treated substantially contemporaneously with (e.g., within 48 hours, within 24 hours, or within 12 hours of) the onset of symptoms of and/or exposure to chlamydia infection. Of course individuals known to have chlamydia infection may receive treatment at any time.

In some embodiments, inventive prophylactic and/or therapeutic protocols involve administering a therapeutically effective amount of one or more inventive immunogenic compositions to a subject such that an immune response is stimulated in both T cells and B cells.

In some embodiments, by combining one or more chlamydia antigens and adjuvants, immune responses (e.g. T cell and/or B cell responses) can be tailored to preferentially elicit the most desirable type of immune response for a given indication, e.g., humoral response, Th1 T cell response, Th17 T cell response, IFN-γ secretion by antigen-specific T cells, cytotoxic T cell response, antibody response, B cell response, innate immune response, or a combination of these responses.

### Immunogenic Compositions

The present invention provides immunogenic compositions (e.g., vaccines) comprising a novel chlamydia antigen, e.g., one or more of a polypeptide antigen selected from Table 1, Table 2, Table 3, or combinations thereof, and one or more pharmaceutically acceptable excipients. In accordance with some embodiments, a method of administering an inventive immunogenic composition to a subject in need thereof is provided. In some embodiments, inventive compositions are administered to humans. For the purposes of the present invention, the phrase “active ingredient” generally refers to an inventive immunogenic composition comprising at least one chlamydia antigen and optionally comprising one or more additional agents, such as an adjuvant.

Although the descriptions of immunogenic compositions provided herein are principally directed to compositions which are suitable for administration to humans, it will be understood by the skilled artisan that such compositions are generally suitable for administration to animals of all sorts. Modification of immunogenic compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the immunogenic compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

The formulations of the immunogenic compositions described herein may be prepared by any method known or hereafter developed in the art of vaccines. In some embodiments, such preparatory methods include the step of bringing the antigen(s) (or nucleic acids encoding the antigens, for nucleic acid based applications) into association with one or more excipients and/or one or more other accessory ingredients, and then, if necessary and/or desirable, shaping and/or packaging the product into a desired single- or multi-dose unit.

An immunogenic composition of the invention may be prepared, packaged, and/or sold in bulk, as a single unit dose, and/or as a plurality of single unit doses. As used herein, a “unit dose” is discrete amount of the immunogenic composition comprising a predetermined amount of the antigen(s).

The relative amounts of the antigen(s), the pharmaceutically acceptable excipient(s), and/or any additional ingredients (e.g., adjuvant) in a composition of the invention will vary, depending upon the identity, size, and/or condition of the subject treated and further depending upon the route by which the composition is to be administered.

Immunogenic formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006; incorporated herein by reference) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the immunogenic composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of immunogenic compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations.

Injectable formulations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong release of an immunogenic composition and stimulate maximal uptake by antigen presenting cells in the vicinity of an injection site, it is often desirable to slow the absorption from subcutaneous or intramuscular injection. This may be accomplished by the use of a liquid suspension of crystalline or amorphous material with poor water solubility. Alternatively, delayed absorption of a parenterally administered drug form may be accomplished by dissolving or suspending the drug in an oil vehicle.

In some embodiments, an immunogenic composition is administered to a mucosal surface. Compositions for rectal or vaginal administration can include suppositories which can be prepared by mixing immunogenic compositions of this invention with suitable excipients such as cocoa butter, polyethylene glycol or a suppository wax, which are solid at ambient temperature but liquid at body temperature and therefore melt in the rectum or vaginal cavity and release antigen.

In some embodiments, an immunogenic composition is administered orally. Solid dosage forms for oral administration include capsules, tablets, pills, powders, and granules. In such solid dosage forms, the antigen can be mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Suitable devices for use in delivering immunogenic compositions by an intradermal route described herein include short needle devices such as those described in U.S. Pat. Nos. 4,886,499; 5,190,521; 5,328,483; 5,527,288; 4,270,537; 5,015,235; 5,141,496; and 5,417,662. Jet injection devices which deliver liquid immunogenic compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate an immunogenic composition in powder form through the outer layers of the skin to the dermis are suitable. Alternatively or additionally, conventional syringes may be used in the classical mantoux method of intradermal administration.

General considerations in the formulation and/or manufacture of pharmaceutical agents may be found, for example, in Remington: The Science and Practice of Pharmacy 21^{st} ed., Lippincott Williams & Wilkins, 2005.

### Administration

In some embodiments, a therapeutically effective amount of an inventive immunogenic composition is delivered to a patient and/or animal prior to, simultaneously with, and/or after exposure to a chlamydia organism or diagnosis with a chlamydial disease, disorder, and/or condition. In some embodiments, a therapeutic amount of an inventive composition is delivered to a patient and/or animal prior to, simultaneously with, and/or after onset of symptoms of a chlamydial disease, disorder, and/or condition. In some embodiments, the amount of an immunogenic composition is sufficient to reduce risk of infection by, or treat, alleviate, ameliorate, relieve, delay onset of, inhibit progression of, reduce severity of, and/or reduce incidence of one or more symptoms or features of the chlamydial disease, disorder, and/or condition.

Immunogenic compositions, according to the method of the present invention, may be administered using any amount and any route of administration effective for treatment. The exact amount required will vary from subject to subject, depending on the species, age, and general condition of the subject, the severity of the infection, the particular composition, its mode of administration, its mode of activity, and the like. The specific effective dose level for any particular subject or organism will depend upon a variety of factors including the immunogenicity of the antigen composition employed; the specific composition employed; the nature of adjuvant used; the age, body weight, general health, sex and diet of the subject; the time of administration, route of administration, and like factors well known in the medical arts.

Immunogenic compositions of the present invention may be administered by any route that elicits an immune response. In some embodiments, an immunogenic composition is administered subcutaneously. In some embodiments, an immunogenic composition is administered intramuscularly. In some embodiments, the immunogenic compositions of the present invention are administered by a variety of routes, including oral, intravenous, intra-arterial, intramedullary, intrathecal, intraventricular, transdermal, interdermal, rectal, intravaginal, intraperitoneal, topical (as by powders, ointments, creams, and/or drops), transdermal, mucosal, nasal, buccal, enteral, sublingual; by intratracheal instillation, bronchial instillation, and/or inhalation; and/or as an oral spray, nasal spray, and/or aerosol.

In certain embodiments, an immunogenic composition of the invention may be administered in amounts that include a protein antigen in ranges of 1 μg-500 μg. In some embodiments, a dose of about 10 μg, 20 μg, 30 μg, 50 μg, or 100 μg is administered to a human.

In some embodiments, an immunogenic composition is administered more than once (e.g., twice, three times, four times, five times). In some embodiments, a boost is given about one week, two weeks, three weeks, one month, three months, six months, one year, or longer after an initial immunization.

### Kits

The present invention provides a variety of kits comprising one or more of the antigens described herein. For example, the invention provides a kit including a novel chlamydia antigen and instructions for use. A kit may include multiple different chlamydia antigens. A kit may include any of a number of additional components or reagents in any combination. All of the various combinations are not set forth explicitly but each combination is included in the scope of the invention.

According to certain embodiments of the invention, a kit may include, for example, (i) an immunogenic composition including at least one of the following chlamydia antigens: CT062, CT572, CT043, CT570, CT177, CT725, CT067, CT476, p6, CT310, or CT638 polypeptide antigens; and (ii) instructions for administering the composition to a subject in need thereof. In some embodiments, the kit further includes an adjuvant.

Kits that include nucleic acids encoding chlamydia antigens are also provided. In certain embodiments, a kit may include, for example, (i) a composition including a nucleic acid encoding a chlamydia antigen; (ii) instructions for use of the nucleic acid compositing (e.g., instructions for expressing the nucleic acid for producing the antigen, or instructions for administering the composition to a subject in need thereof to elicit a response against chlamydia).

Instructions included with kits may, for example, include protocols and/or describe conditions for production of immunogenic compositions and/or administration of immunogenic compositions, to a subject in need thereof, etc. Kits generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### EXEMPLIFICATION

### Example 1

### Peripheral Blood Mononuclear Cells and Plasma from Women with a Clinical History of Chlamydia trachomatis Infection are Used to Identify Chlamydia Protein Antigens

### Isolation and Screening of Chlamydia-Specific T Cells

Heparinized whole blood was collected from women with documented Chlamydia trachomatis exposure or a clinical history of genital infection. Donors were classified as “protected” if they were repeatedly exposed to the bacteria but not infected, or if they became infected but cleared their infection without medical intervention. Donors were classified as “unprotected” if they were persistently infected or if their infections progressed to more severe complications such as pelvic inflammatory disease. Peripheral blood mononuclear cells (PBMC) were isolated from the blood samples by Ficoll density gradient centrifugation and cyropreserved for use on a later date. When the PBMC were thawed, CD14⁺ monocytes were separated using antibody coated magnetic beads and placed into culture with GM-CSF and IL-4 cytokines to derive them into dendritic cells (MDDC). Additionally, T cells were enriched from PBMC by magnetic bead depletion using the Miltenyi Pan T sorting kit following the manufacturer's instructions. The resulting enriched T cell population was then sorted using antibody-conjugated magnetic beads specific for CD4⁺ T cells (Miltenyi). The CD4 negative population was considered to be CD8⁺. (In some cases, the PBMC depleted of T cells were cyropreserved.) Both T cell subsets were non-specifically expanded in vitro using magnetic beads coated with anti-CD3 and anti-CD28 antibodies (Dynal T Cell Expander). The T cells were maintained at 10⁶ cells/mL in AIM-V-5% (AIM-V, 5% FCS, Non-essential Amino Acids, Sodium Pyruvate, L-Glutamine, and beta-mercaptoethanol) plus recombinant IL-2. After sufficient T cell numbers were achieved, the CD3/CD28 magnetic beads were removed from culture, and the enriched and expanded CD4⁺ and CD8⁺ T cells were separately screened using a chlamydia ORFeome library to determine which antigens naturally induced T cell responses. T cell screening required the co-culture of expanded T cells with autologous antigen presenting cells (APC) that were pulsed with the proteomic library. APC were pulsed with induced bacteria from the proteomic library at a 100:1 ratio of induced bacteria to APC. There were two methods of preparing autologous APC for T cell screens. Method 1 plated 10⁴ MDCC per well in 384-well flat bottom plates. Method 2 plated 10⁵ APC per well comprised of MDCC and thawed T cell-depleted PBMC in 96-well round bottom plates. For both methods, screen plates containing APC and library-expressing bacteria were placed in a 37° C., 5% CO₂ humidified incubator. After a two-hour incubation, the APC were washed with PBS and then fixed with 1% paraformaldehyde (PFA). The fixed APC were washed extensively, then expanded T cells were added to the pulsed, fixed APC and the plates returned to a 37° C., 5% CO₂ humidified incubator. Optimally, 4×10⁴ T cells were added to the 10⁴ pulsed MDCC plated in each well of the 384-well plates described in Method 1. Alternatively, up to 10⁵ T cells were added to the 10⁵ pulsed APC plated in each well of the 96-well plates described in Method 2. After 24 hours of co-culture, T cell responses were monitored by measuring interferon gamma (IFN-γ) in the cell-free supernatants by ELISA (BD OptEIA kit).

Identification of Chlamydia Protein Antigens that Induce T Cell Responses

Over 110 samples from human subjects were screened against the library as described above. Library proteins that induced IFN-γ responses that exceeded twice the mean average deviation of the median of the data after background correction were considered to be positive in this screen. To validate the identity of each identified antigen, plasmid DNA from the library stock was purified and sequenced. The primer used for sequencing was a consensus primer located within the plasmid, upstream of each clone. Alignments were performed using the nucleotide BLAST feature of the NCBI website on the Internet at the following address: blast.ncbi.nlm.nih.gov/Blast.cgi. Listed sequences are those of the annotated genes, as found in GenBank, corresponding to the isolated clones.

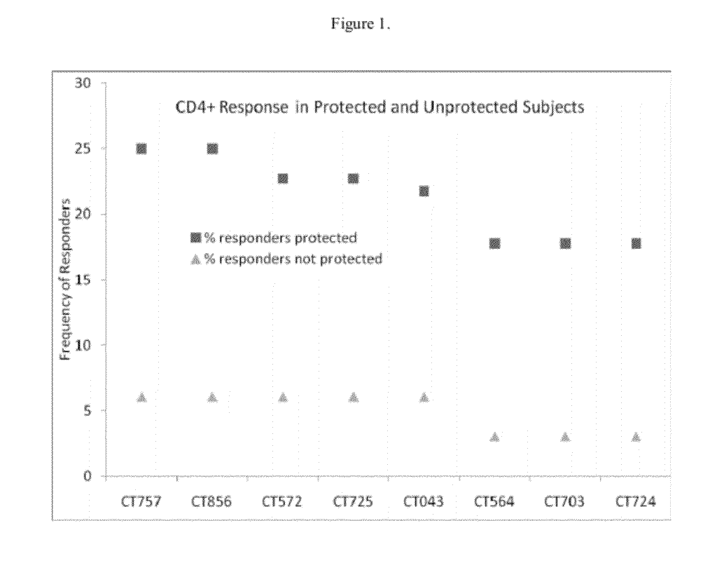

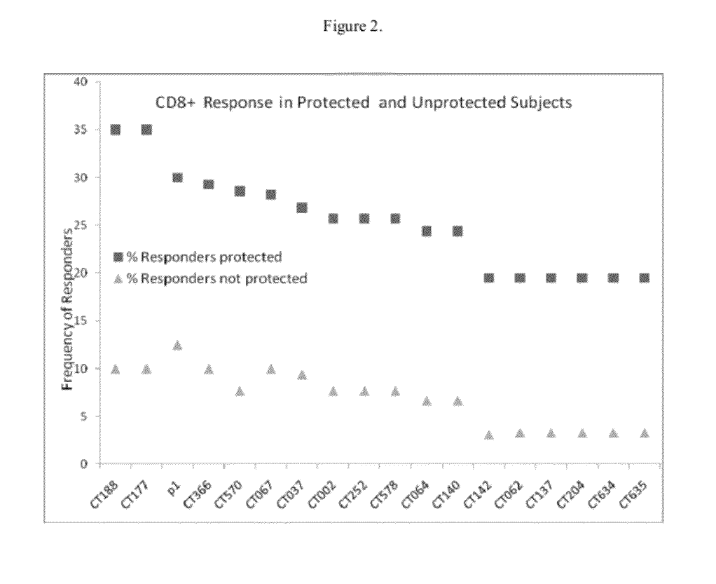

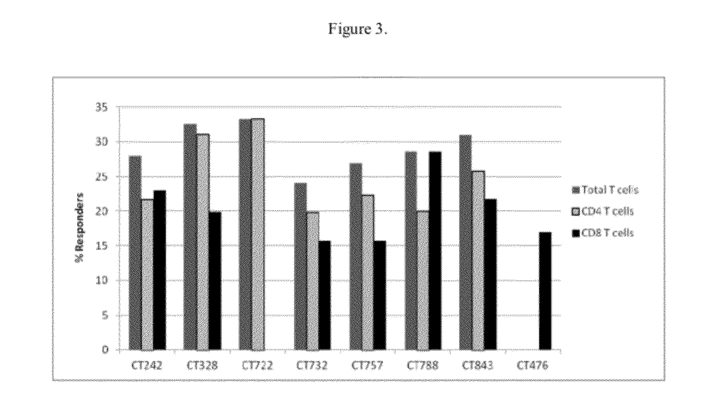

FIGS. 1, 2, and 3 depict exemplary graphs illustrating the frequency with which identified antigens were recognized by, respectively, CD4⁺ and CD8⁺ T cells obtained from protected and unprotected donors. Based on evaluation of negative controls, donor and plate variation, a donor was classified as a “responder” if the fold ratio of the value over negative control was greater than 1.63 (CD4⁺) or 1.66 (CD8⁺). Percent responders >10% indicated a higher number of responders than due to chance alone. Statistical significance was reached when the percent responders was >15% (all donors, including negative controls), or approximately 19% (protected and unprotected donors). FIG. 1 and FIG. 2 depict separate exemplary results for protected and unprotected donors. Four C. trachomatis proteins induced CD4⁺ or CD8⁺ T cell responses (two clones each, respectively) with statistically greater frequency in protected compared to unprotected donors, with a p-value of 0.05. An additional 16 clones induced CD8⁺ T cell responses and 6 clones induced CD4⁺ T cell responses with greater frequency in protected donors, with a p-value of 0.1. Antigens that are represented with greater frequency in donors who were clinically protected from their infection are correlated with protective immunity and the best candidates for vaccine formulation. FIG. 3 depicts an exemplary result illustrating CD4⁺, CD8⁺, and combined T cell responses for all donors (protected and unprotected). Antigens represented at the highest overall frequency, whether or not represented at statistically higher frequency in protected donors, are also attractive candidates for vaccine, diagnostic and prognostic applications.

Identification of Chlamydia Protein Antigens that Induce B Cell Responses

The plasma fraction of heparinized whole blood from women with documented Chlamydia trachomatis exposure or a clinical history of genital infection, as described in the present Example, was collected by centrifugation and stored at −80° C. until used. Each clone of a chlamydia ORFeome library in E. coli was induced for 24 hours to allow for protein expression. Bacteria were pelleted, resuspended in lysis buffer, and arrayed in 96-well plates. Following two rounds of extraction with urea, supernanants containing the proteins were diluted 1:2 with 20 mM Tris buffer and each protein concentration was determined by Coomasie staining. The concentration of each protein was adjusted to 400 μg/mL by the addition of 4 mM urea/Tris buffer. The plates were then sealed and shipped for printing onto microarrays (Gentel Biosciences, Inc.). The protein microarrays were probed with plasma samples of subjects recruited for T cell screens above. An antibody specific for human IgG was used to probe the bound plasma samples for protein specific antibody and detected by chromogenic substrate. Responses were considered positive if the signal was statistically significantly above the background value of negative controls. Two criteria were used for selection: the first was overall frequency of responses across all cohorts and the second was responses with statistically greater frequency in protected subjects as compared to unprotected donors, with a p-value of <0.05.

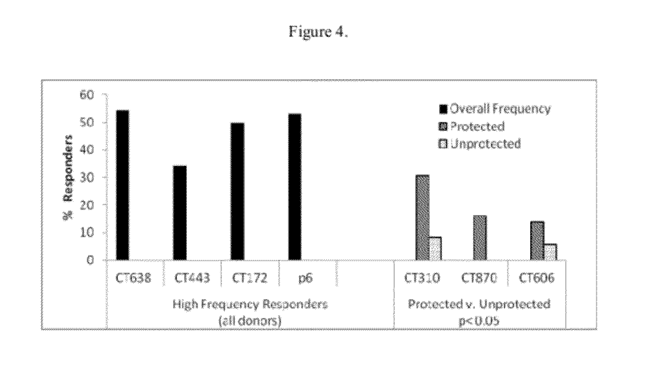

FIG. 4 depicts an exemplary result illustrating the frequency with which chlamydia antigens were bound by IgG present in donor sera, i.e. have elicited a donor B cell response. The left side of the panel displays chlamydia antigens detected by IgG with overall highest frequency across all donors (protected and unprotected). The right side of the panel displays chlamydia antigens detected by IgG with statistically greater frequency in protected donors as compared to unprotected donors.

### Example 2

### Identified Chlamydia Protein Antigens are Immunogenic in Mice

### Immunization Protocol

Mice were immunized subcutaneously in the scruff of the neck with a 100 μl injection of 5 μg antigen plus adjuvant (12 μg dose of an ISCOM matrix with a 91:9 mixture of Quillaja saponin matrix A and matrix C) in saline. The mice received two injections, 21 days apart. Seven days after the final injection, mice were euthanized, and blood and tissues harvested for further analysis.

### Assay for Ex Vivo, T Cell-Mediated IFN-γ Responses

An ex vivo IFN-γ ELISPOT was used to quantify T cell responses. CD4⁺ and CD8⁺ T cells were enriched from mouse splenocytes using magnetic beads, starting from mouse spleens harvested above. Membrane plates were prepared by coating overnight with capture antibody specific for IFN-γ and subsequently blocked with supplemented medium for a minimum of 2 hours at 37° C. APCs were prepared by pulsing naïve T-depleted splenocytes with antigen for 2 hours at 37° C. For CD4⁺ ELISPOTs, APCs were pulsed with whole protein. For CD8⁺ ELISPOTs, ISCOM matrix at a concentration of 20 μg/mL was added to the whole protein to facilitate antigen uptake and processing. The APCs and T cells were added to appropriate wells of the pre-coated plates. A negative control was APCs incubated for 2 hours at 37° C. with no additional antigen, and a positive control was T cells incubated with phorbol myristate acetate (PMA) and ionomycin. The plates were allowed to incubate for 18 hours at 37° C. under 5% CO₂. The spots were visualized using a secondary biotinylated antibody specific for IFN-γ, horseradish peroxidase (HRP) and 3-amino-9-ethylcarbazole (AEC) substrate.

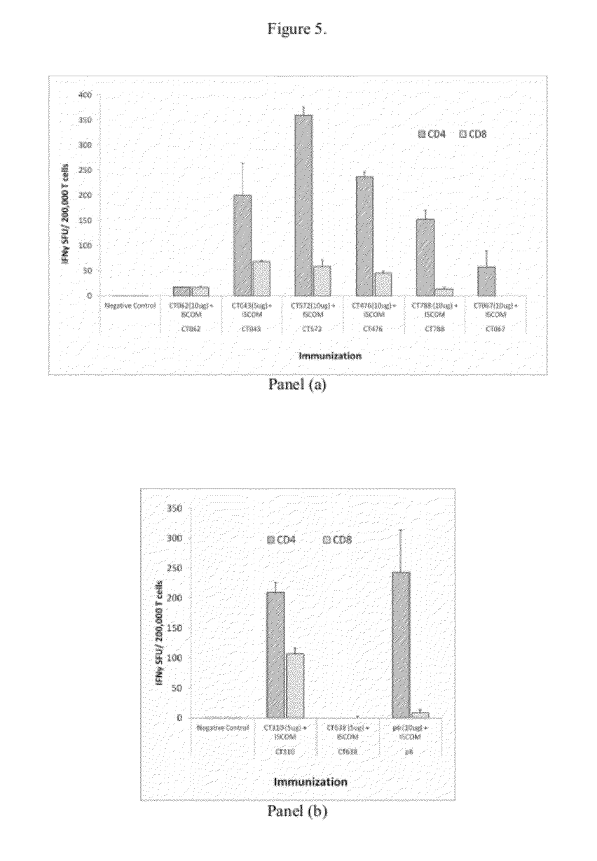

FIG. 5 depicts an exemplary result illustrating IFN-γ levels induced ex vivo in CD4⁺ and CD8⁺ T cells from mice immunized with the indicated chlamydia protein antigen and re-stimulated in vitro with the same antigen. FIG. 5A depicts an exemplary result illustrating antigens that were originally identified through T cell responses. FIG. 5B depicts an exemplary result illustrating antigens that were originally identified through B cell responses, demonstrating that these antigens can in some cases also elicit robust T cell responses.

### Assay for B Cell-Mediated Antibody Responses

Antigen-specific serum antibody titers of immunized mice were determined by direct protein ELISA. Blood was collected 7 days post last injection by terminal cardiac puncture. The sera were processed and stored at −80° C. ELISA plates were coated overnight at 4° C. with 5 μg of whole protein in 0.1 M carbonate buffer, pH 9.5. Plates were washed with TBS+0.05% Tween-20 (TBS-T) and blocked with TBS-T+1% bovine serum albumin for 1 h. Serum samples were serially diluted and incubated in the antigen-coated wells for 2 hours at room temperature. Plates were washed and probed for 1 h with goat anti-mouse alkaline-phosphatase (AP)-conjugated anti-IgG at a 1:10,000 dilution. Detection of AP activity was achieved by the addition of p-Nitrophenyl phosphate (pNPP; Sigmafast, Sigma-Aldrich), and the reaction stopped with 3N NaOH and absorbance read at 405 nm. Endpoint titers were calculated by extrapolation of the linear portion of the serial dilutions and defining the endpoint as the dilution at which the linear portion of the curve intersects with the background cut-off. The cut-off used for data calculation was 2 times the value of the negative control serum from a naïve mouse.

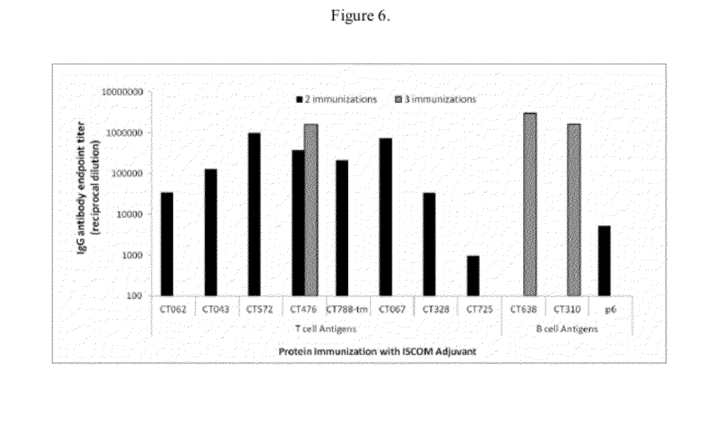

FIG. 6 depicts an exemplary result illustrating IgG antibody titers against the indicated chlamydia antigens, following immunization with the same antigen. Results shown in the left side of the panel demonstrate that antigens originally identified through T cell responses (e.g. FIGS. 1, 2 and 3) can in some cases also elicit robust B cell responses.

### Example 3

### Mice Immunized with Identified Chlamydia Protein Antigens are Protected against Chlamydia trachomatis Challenge

### Immunization Protocol

C57BL/6 mice (8 per group) were immunized subcutaneously in the scruff of the neck with a 100 μl injection of 5 μg antigen plus adjuvant (24 μg dose of an ISCOM matrix with a 91:9 mixture of Quillaja saponin matrix A and matrix C) in saline. The mice received two injections, 21 days apart. Depo-Provera (1.25 mg) was administered subcutaneously at 10 and 3 days prior to intravaginal challenge to synchronize estrus.

Intravaginal Infection with Chlamydia trachomatis

Chlamydia trachomatis serovar D (D/UW-3/CX) bacteria were propagated in McCoy cells, and elementary bodies were purified by RenoCal-76 gradient centrifugation and stored in sucrose phosphate (SPG) buffer. The mice were challenged seven days after the last immunization by intravaginal deposition of 0.5−1×10⁶ IFU Chlamydia trachomatis serovar D elementary bodies directly onto the ectocervix with a positive displacement pipet.

Determination of Chlamydia trachomatis Burden in Ectocervix, Post-Infection

Samples of the ectocervix and vaginal vault of immunized and challenged mice were collected 3, 7, 10, 14, and 21 days post-infection. Chlamydia present in the samples were quantified by direct culture on McCoy cell monolayers. Serial dilutions of swab samples in SPG buffer were added to confluent McCoy cell monolayers and centrifuged at 2400 RPM for 1 h at 37° C. Supernatants were removed and replaced with cRPMI containing 1 μg/mL cyclohexamide and incubated for 44 h at 37° C. The monolayers were fixed with 100% methanol, stained with FITC-labeled anti-chlamydia antibody (Millipore), and inclusions were counted for determination of IFU.

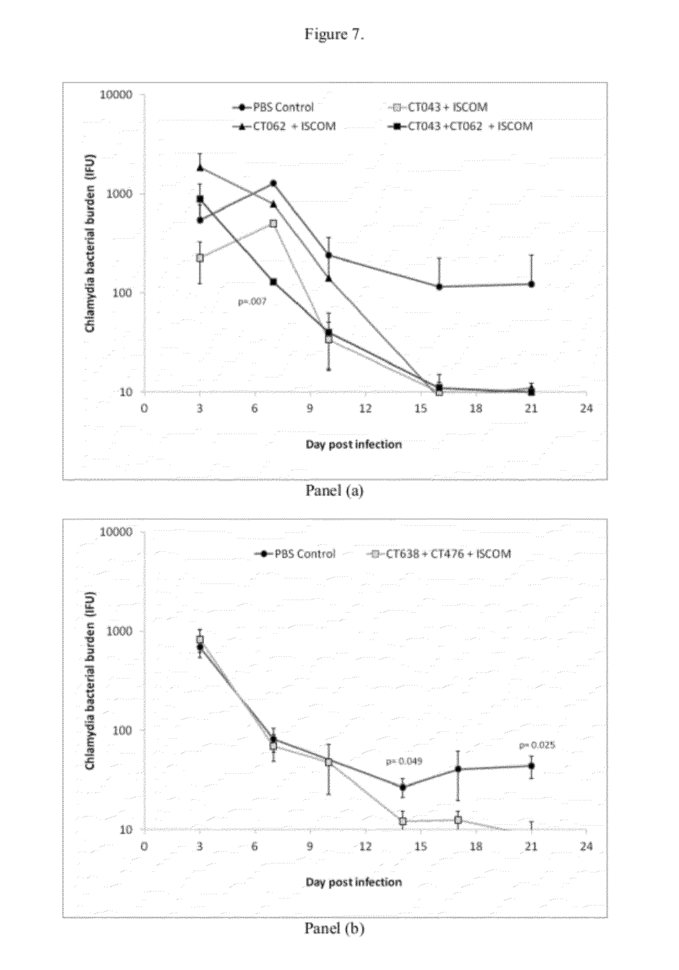

FIG. 7 depicts an exemplary result illustrating reduction of ectocervical chlamydia burden in mice immunized with the indicated chlamydia protein antigens and subsequently intravaginally infected with Chlamydia trachomatis. FIG. 7A depicts an exemplary result for representative chlamydia protein antigens CT062, CT043, and for the combination CT062+CT043. FIG. 7B depicts an exemplary result for representative chlamydia protein antigen combination CT638+CT476.

Determination of Chlamydia trachomatis Burden in Upper Reproductive Tract, Post-Infection

Oviducts and ovaries were collected from immunized and challenged mice at day 21 post-infection. Chlamydia, living and dead, present in whole oviducts and ovaries were detected by real-time quantitative PCR. The oviducts and ovaries were digested overnight at 56° C. in tissue lysis buffer containing 0.6 mg Proteinase K. DNA was extracted using the QIAamp DNA Mini Kit (Qiagen) according to manufacturer's instructions. Extracted DNA was subjected to PCR with primers specific for Chlamydia trachomatis 16SrRNA gene. Briefly, 154, of extracted DNA was processed in a 20 uL reaction volume containing 0.8 uM of each primer and 1 U of Taq polymerase. Amplifications were carried out in a StepOnePlus Real-Time PCR system (Applied Biosystems). The gene copy number was determined by extrapolation using a standard curve of Chlamydia 16s rRNA purified plasmid of known copy number.

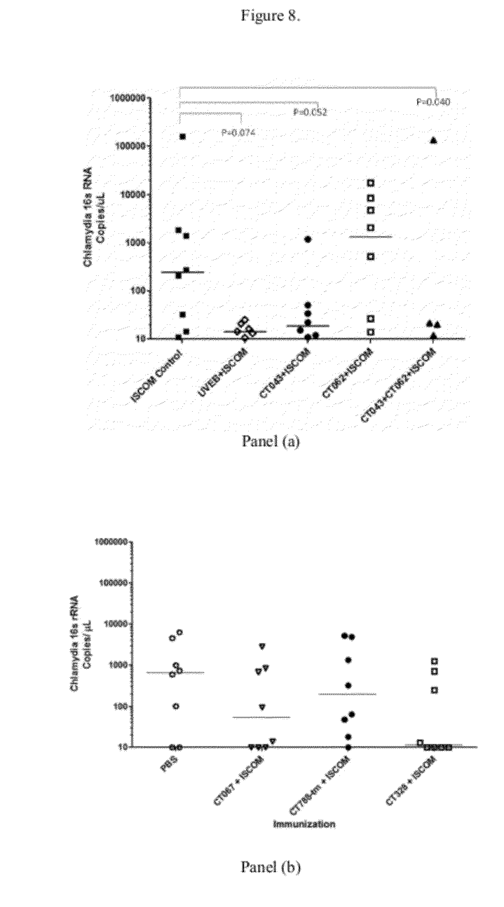

FIG. 8 depicts an exemplary result illustrating reduction of upper reproductive tract chlamydia burden in mice immunized with the indicated chlamydia protein antigens and subsequently intravaginally infected with Chlamydia trachomatis. FIG. 8A depicts an exemplary result for representative chlamydia protein antigens CT062, CT043, and for the combination CT062+CT043. UVEB indicates responses from mice immunized with the positive control, UV-inactivated whole Chlamydia trachomatis elementary bodies. FIG. 8B depicts an exemplary result for representative chlamydia protein antigens CT067, CT0788tm, and CT328.

### Example 4

### Subsequent to Infection with Chlamydia trachomatis, Lymphatic and Splenic T Cells are Primed to Respond to Identified Chlamydia Protein Antigens

### Assay for Lymphatic and Splenic T Cell-Mediated IFN-γ Responses, Post-Infection

Unimmunized mice were intravaginally infected with 1×10⁶ IFU purified Chlamydia trachomatis serovar D elementary bodies as described above. Lateral iliac, aortic lumbar and sacral draining lymph nodes (DLN) and spleens were harvested 7-14 days post-infection. Antigen specific T cell responses following stimulation with identified chlamydia protein antigens were determined by ELISPOT assay on sorted CD4⁺ or CD8⁺ T cells as described under Example 2 above.

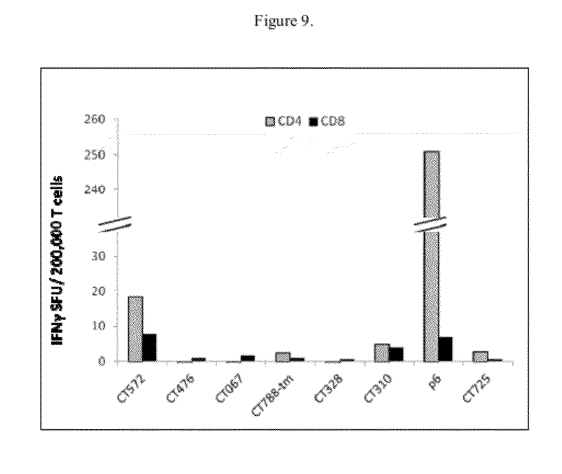

FIG. 9 depicts an exemplary result illustrating induction of IFN-γ in CD4⁺ and CD8⁺ T cells harvested from the spleens of infected mice and stimulated with the indicated chlamydia protein antigens. Results indicate that infection with Chlamydia trachomatis can prime T cells that are specific for the identified antigens, and that can be the target of protective T cells upon re-challenge.

SEQUENCES.

SEQ ID: 1 CT062 polypeptide (412 amino acids; GenBank AAC67653.1)
MQQLIDNLKKRGILDNSSAGLESLTVPVSAYLGFDPTAPSLHIGHWIGICFLRRLAAYGITPVALVGGATGMIGD
PSGKSVERSLLDQAQVLDNSKKIAAALASYLPGIRIVNNADWLGSLSMVDFLRDVGKHFRLGSMLAKDVVKQRVY
SEEGISYTEFSYLLLQSYDFAHLFKEHNVVLQCGGSDQWGNITSGIDYIRRRGLGQAYGLTYPLLTDSKGKKIGK
TESGTIWLDPALTPPYELFQYFLRLPDQEISKVMRTLTLLDNEEIFALDERLTSDPQAVKKYIAEVIVKDVHGSE
GLAQAQAATESFFASKGKSITEAELVALVESGVGVKVARADLIGKRWLDIVVELGFCSSRGQARRLIQQRGLYIN
QEPLADEQSILDGTQLCFDRYVLLSQGKRKKQVIDLN
SEQ ID: 2 CT062 DNA

| | |
|---|---|
| 1 | ATGCAACAGT TAATCGATAA CCTTAAGAAA CGGGGTATTC TAGATAATTC TTCTGCAGGA |
| 61 | TTAGAAAGTT TAACAGTTCC TGTTTCTGCC TATTTAGGGT TCGATCCAAC TGCGCCTTCT |
| 121 | TTACACATAG GACATTGGAT TGGAATTTGT TTTTTGCGTC GATTAGCAGC ATATGGAATC |
| 181 | ACTCCTGTTG CTCTTGTTGG CGGAGCTACC GGAATGATCG GAGATCCTTC TGGTAAAAGT |
| 241 | GTGGAGCGTT CATTACTAGA TCAGGCACAG GTGCTTGATA ATAGTAAGAA AATAGCGGCT |
| 301 | GCTCTTGCTA GCTATCTTCC TGGTATCCGT ATTGTGAATA ATGCGGATTG GCTAGGATCT |
| 361 | TTAAGTATGG TGGATTTTTT AAGAGATGTT GGGAAGCATT TTCGTTTAGG TTCTATGTTA |
| 421 | GCTAAAGACG TAGTGAAGCA GCGAGTCTAT TCTGAAGAGG GAATTAGCTA CACTGAGTTC |
| 481 | AGTTATTTAT TGCTGCAGTC TTATGATTTT GCACATCTCT TTAAAGAGCA TAATGTTGTA |
| 541 | TTACAGTGTG GAGGGAGTGA TCAGTGGGGG AATATTACTT CGGGGATTGA TTATATCCGT |
| 601 | CGAAGAGGAC TAGGGCAGGC TTATGGTCTA ACCTATCCTT TGCTCACTGA TAGCAAAGGG |
| 661 | AAGAAAATAG GGAAGACGGA GTCTGGAACT ATCTGGCTGG ATCCAGCGTT AACTCCTCCT |
| 721 | TATGAACTAT TCCAATATTT CTTACGCTTG CCAGATCAAG AAATCTCCAA AGTAATGAGA |
| 781 | ACTCTTACTC TTTTGGATAA CGAAGAAATT TTTGCTCTTG ATGAGCGTTT GACTAGTGAT |
| 841 | CCACAAGCTG TGAAGAAATA CATTGCGGAA GTGATCGTTA AAGATGTTCA TGGTTCTGAG |
| 901 | GGATTAGCTC AGGCTCAAGC CGCAACCGAA AGCTTTTTTG CTAGTAAGGG AAAGAGTATT |
| 961 | ACAGAAGCAG AACTAGTAGC GTTAGTAGAG TCAGGTGTTG GCGTTAAAGT AGCTCGAGCA |
| 1021 | GATTTAATAG GGAAACGCTG GTTAGATATC GTTGTGGAAC TAGGCTTTTG TTCCTCAAGA |
| 1081 | GGACAAGCTA GAAGACTCAT TCAACAGCGA GGTCTGTACA TCAATCAGGA GCCTTTGGCC |
| 1141 | GATGAACAGA GTATATTAGA CGGGACTCAG TTGTGTTTCG ATCGTTATGT TTTGTTGTCC |
| 1201 | CAAGGGAAAA GAAAAAAACA AGTGATAGAT CTTAATTAG |

SEQ ID: 3 CT572 polypeptide (760 amino acids; GenBank AAC68174.1)
MKNILGYGFLGTFCLGSLTVPSFSITITEKLASLEGKTESLAPFSHISSFNAELKEANDVLKSLYEEALSLRSRG
ETSQAVWDELRSRLIGAKQRIRSLEDLWSVEVAERGGDPEDYALWNHPETTIYNLVSDYGDEQSIYVIPQNVGAM
RITAMSKLVVPKEGFEECLSLLLMRLGIGIRQVSPWIKELYLTNREESGVLGIFGSRQELDSLPMTAHIAFVLSS
KNLDARADVQALRKFANSDTMLIDFIGGKVWLFGAVSEITELLKIYEFLQSDNIRQEHRIVSLSKIEPLEMLAIL
KAAFREDLAKEGEDSSGVGLKVVPLQNHGRSLFLSGALPIVQKAIDLIRELEEGIESPTDKTVFWYHVKHSDPQE
LAALLSQVHDIFSNGAFGASSSCDTGVVSSKAGSSSNGLAVHIDTSLGSSVKEGSAKYGSFIADSKTGTLIMVIE
KEALPKIKMLLKKLDVPKKMVRIEVLLFERKLSNQRKSGLNLLRLGEEVCKQGTQAVSWASGGILEFLFKGGAKG
IVPSYDFAYQFLMAQEDVRINASPSVVTMNQTPARIAIVEEMSIVVSSDKDKAQYNRAQYGIMIKILPVINIGEE
DGKSFITLETDITFDSTGRNHADRPDVTRRNITNKVRIQDGETVIIGGLRCNQTMDSRDGIPFLGELPGIGKLFG
MDSASDSQTEMFMFITPKILDNPSETEEKLECAFLAARPGENDDFLRALVAGQQAAKQAIERKESTVWGEESSGS
RGRVEYDGRE
SEQ ID: 4 CT572 DNA

| | |
|---|---|
| 1 | TTATTCCCGT CCATCATACT CCACCCTTCC TCGAGAGCCG GAGGATTCTT CTCCCCATAC |
| 61 | GGTAGACTCT TTTCTTTCTA TAGCCTGTTT AGCAGCCTGC TGTCCTGCTA CTAAAGCTCT |
| 121 | GAGGAAATCA TCGTTCTCCC CGGGGCGAGC AGCCAGGAAA GCACATTCTA ATTTTTCTTC |
| 181 | TGTCTCACTA GGATTATCCA AAATCTTCGG AGTGATAAAC ATAAACATCT CTGTTTGTGA |
| 241 | GTCCGAAGCA GAATCCATAC CAAATAATTT TCCTATTCCT GGCAACTCTC CTAAAAATGG |
| 301 | AATCCCGTCA CGAGAATCCA TAGTTTGATT ACAACGAAGC CCCCCAATAA TGACCGTTTC |
| 361 | GCCATCTTGA ATCCGAACCT TGTTCGTAAT ATTTCTGCGT GTAACATCGG GACGATCCGC |
| 421 | ATGATTTCTC CCAGTCGAAT CAAACGTGAT GTCGGTCTCT AAAGTAATAA AGCTCTTCCC |
| 481 | ATCCTCTTCT CCGATATTAA TAACGGGAAG AATCTTAATC ATAATCCCGT ATTGAGCTCG |
| 541 | ATTGTATTGG GCTTTATCCT TATCAGAAGA AACTACAATT GACATTTCTT CCACAATCGC |
| 601 | AATTCTCGCC GGGGTTTGGT TCATAGTCAC GACGGAAGGA CTTGCATTAA TACGGACATC |
| 661 | CTCTTGCGCC ATGAGAAACT GATAAGCAAA GTCATAACTA GGAACAATCC CTTTTGCTCC |
| 721 | ACCTTTGAAC AGGAACTCCA GAATGCCCCC ACTTGCCCAC GAAACGGCTT GCGTTCCCTG |
| 781 | CTTACAAACC TCTTCTCCTA AACGCAATAG GTTCAATCCA GATTTACGTT GATTGGATAG |
| 841 | TTTTCTTTCA AAAAGCAGAA CCTCTATACG TACCATTTTT TTGGGCACAT CCAGTTTCTT |
| 901 | CAACAACATC TTGATCTTGG GTAAAGCTTC TTTCTCAATA ACCATAATCA AGGTTCCGGT |
| 961 | CTTGGAATCT GCAATAAAAC TCCCATATTT CGCAGAACCT TCTTTTACGG AGCTCCCCAG |
| 1021 | CGACGTATCT ATATGTACCG CTAATCCATT CGAAGAGGAT CCCGCTTTAC TTGAGACTAC |
| 1081 | GCCAGTATCA CAACTACTAG ATGCCCCAAA AGCACCATTT GAGAAAATAT CATGTACTTG |
| 1141 | AGAAAGAAGC GCTGCAAGCT CCTGAGGATC TGAGTGTTTG ACATGATACC AAAATACCGT |
| 1201 | TTTGTCGGTA GGGCTCTCTA TCCCCTCTTC TAGTTCCCGA ATAAGATCTA TTGCCTTCTG |
| 1261 | AACGATGGGA AGAGCTCCAC TTAAGAAAAG CGAGCGTCCA TGGTTTTGTA AAGGGACCAC |
| 1321 | TTTTAATCCC ACTCCAGAAG AATCTTCTCC CTCTTTAGCT AAATCTTCTC GGAAAGCTGC |
| 1381 | TTTCAAAATA GCCAGCATTT CTAAGGGTTC TATTTTTGAT AAAGAAACAA TGCGATGCTC |
| 1441 | TTGTCGAATG TTGTCTGATT GTAAGAATTC ATAGATTTTA AGGAGCTCGG TAATCTCGCT |
| 1501 | GACAGCTCCA AATAACCAAA CTTTCCCCCC TATAAAATCA ATTAACATGG TATCGCTATT |
| 1561 | TGCGAACTTG CGCAAAGCTT GTACATCCGC TCGTGCATCT AAATTTTTAG AAGAAAGTAC |
| 1621 | AAAAGCAATA TGTGCCGTCA TAGGCAAGCT ATCTAGCTCT TGTCTAGATC CAAAGATACC |
| 1681 | TAAAACACCA GACTCTTCCC TATTAGTTAA ATACAGCTCC TTAATCCAAG GACTAACCTG |
| 1741 | TCTGATCCCA ATACCCAGCC GCATTAAAAG CAAAGACAAA CATTCCTCAA ATCCTTCTTT |
| 1801 | AGGGACCACT AGCTTAGACA TGGCTGTGAT ACGCATCGCC CCAACATTTT GAGGAATCAC |
| 1861 | ATAGATACTC TGTTCATCTC CGTAATCACT GACCAGATTA TAAATCGTAG TTTCTGGATG |
| 1921 | ATTCCAAAGG GCATAGTCTT CGGGATCCCC CCCCCTTTCT GCAACCTCTA CTGACCATAA |
| 1981 | ATCTTCCAAT GAACGTATCC GTTGTTTAGC GCCGATCAAT CGGCTTCGCA ACTCGTCCCA |
| 2041 | TACCGCCTGC GAAGTCTCTC CTCGAGAACG GAGAGACAAA GCTTCTTCGT ATAAAGATTT |
| 2101 | GAGAACATCA TTTGCCTCTT TCAATTCAGC ATTAAAAGAT GAAATATGCG AAAAAGGGGC |
| 2161 | TAGCGATTCC GTTTTTCCTT CTAGAGAAGC CAATTTTTCT GTAATCGTGA TGGAAAAACT |
| 2221 | AGGAACCGTC AAACTTCCCA AACAAAAAGT CCCTAGAAAC CCATAGCCCA AAATATTTTT |
| 2281 | CAC |

SEQ ID: 5 CT043 polypeptide (167 amino acids; GenBank AAC67634.1)
MSRQNAEENLKNFAKELKLPDVAFDQNNTCILFVDGEFSLHLTYEEHSDRLYVYAPLLDGLPDNPQRRLALYEKL
LEGSMLGGQMAGGGVGVATKEQLILMHCVLDMKYAETNLLKAFAQLFIETVVKWRTVCSDISAGREPTVDTMPQM
PQGGGGGIQPPPAGIRA
SEQ ID: 6 CT043 DNA

| | |
|---|---|
| 1 | TTATGCACGG ATTCCTGCTG GAGGAGGTTG AATTCCTCCG CCACCCCCTT GAGGCATTTG |
| 61 | TGGCATGGTA TCAACAGTGG GTTCTCGTCC AGCGCTGATA TCAGAACAAA CAGTTCGCCA |
| 121 | TTTCACAACG GTTTCAATAA AAAGCTGTGC AAAAGCTTTG AGTAGGTTGG TCTCTGCATA |
| 181 | CTTCATGTCT AACACGCAGT GCATTAAGAT CAACTGTTCC TTAGTAGCGA CTCCTACCCC |
| 241 | TCCACCAGCC ATTTGGCCTC CGAGCATAGA GCCTTCTAAC AACTTCTCAT ATAGAGCTAA |
| 301 | CCTTCTTTGC GGATTGTCTG GCAGTCCGTC AAGAAGAGGT GCGTAAACAT AAAGGCGATC |
| 361 | AGAGTGTTCT TCGTAGGTCA GGTGAAGAGA AAACTCTCCA TCAACAAACA AAATGCACGT |
| 421 | ATTATTCTGA TCGAAGGCCA CGTCGGGGAG TTTAAGCTCT TTAGCAAAAT TTTTTAGATT |
| 481 | TTCCTCAGCA TTCTGCCTGG ACAT |

SEQ ID: 7 CT570 polypeptide (391 amino acids; GenBank AAC68172.1)
MARFLCTYLDQSEKKRRSFVEAFHQREARELLAAQGAHILDIRKVRERNYRVTTTELVIFTKQLVLLLRSGISLY
DALTSLRDQYQGRALAGVLTSLMEALRSGGVFSEALARFPHIFDSFYQNSVRSGESIGNLEGALMNIIKVLEEKE
KLSKSLAAALSYPVILLVFSCAVVVFFLIGVIPTLKETFEDMEMTRLTKAVFSCSTWFCRYKFLVLLGGIGGAIS
LRIVWKKRIGKRTLEAIIKKIPILRSLVIKIGFCRFCSVTSAVLQGGGNLIEALTLGCEAVSQDFLREELQEVIQ
AVVRGGSLSRELSHRTWTPKLVIGMVALGEESGDLAVVFAHVAQIYNEDIQRVLTWVTAWCQPIVLVLLGGFIGL
IMLSILLPLTSGIQTF
SEQ ID: 8 CT570 DNA

| | |
|---|---|
| 1 | TTAAAACGTT TGAATACCGC TTGTTAACGG AAGAAGGATT GATAACATAA TCAATCCAAT |
| 61 | AAAACCGCCT AGCAACACAA GAACTATGGG CTGACACCAG GCAGTTACCC AAGTCAATAC |
| 121 | CCTTTGAATA TCCTCGTTAT AAATTTGCGC GACATGCGCG AATACCACCG CAAGATCCCC |
| 181 | GGATTCTTCT CCTAGAGCAA CCATCCCAAT CACCAGTTTT GGCGTCCATG TACGATGAGA |
| 241 | TAGCTCACGA CTCAAAGATC CTCCACGAAC AACTGCTTGG ATCACTTCTT GTAGCTCTTC |
| 301 | GCGCAAAAAG TCTTGTGATA CGGCCTCGCA TCCTAATGTC AGAGCTTCGA TCAAATTCCC |
| 361 | GCCTCCTTGC AAAACAGCAG ATGTGACGGA ACAAAATCGA CAAAATCCTA TTTTAATCAC |
| 421 | CAGACTACGC AAAATAGGGA TCTTCTTGAT AATTGCCTCT AGAGTCCTTT TCCCTATCCG |
| 481 | TTTTTTCCAG ACTATGCGTA GGGATATCGC TCCACCTATT CCTCCCAGCA AAACAAGAAA |
| 541 | CTTGTACCTA CAAAACCATG TACTGCACGA GAAAACAGCT TTTGTGAGCC TTGTCATCTC |
| 601 | CATATCTTCA AAAGTTTCTT TCAATGTAGG AATGACCCCT ATTAGAAAGA ACACCACAAC |
| 661 | AGCACAAGAA AATACCAATA AGATCACTGG ATAACTCAAT GCTGCAGCAA GACTTTTGGA |
| 721 | TAGTTTTTCC TTCTCTTCCA ACACTTTAAT AATATTCATT AAAGCGCCTT CTAGATTCCC |
| 781 | AATACTCTCT CCAGAACGCA CACTATTCTG ATAAAAAGAA TCAAAAATAT GCGGGAACCT |
| 841 | CGCTAGAGCT TCTGAAAAGA CCCCACCGGA ACGTAGAGCT TCCATCAAAG AAGTGAGAAC |
| 901 | CCCAGCCAGC GCACGTCCCT GATACTGATC TCGCAATGAA GTCAAAGCAT CGTATAAGGA |
| 961 | GATCCCCGAT CGTAATAATA ACACTAATTG CTTAGTAAAA ATAACCAGCT CTGTAGTTGT |
| 1021 | GACACGGTAG TTTCTCTCTC GCACCTTTCG AATGTCCAGA ATGTGAGCTC CTTGAGCAGC |
| 1081 | AAGAAGCTCT CTTGCCTCTC GCTGATGGAA AGCCTCTACA AAAGAACGTC GTTTTTTCTC |
| 1141 | GGACTGATCA AGATATGTAC AAAGAAACCT AGCCAT |

SEQ ID: 9 CT177 polypeptide (238 amino acids; GenBank AAC67768.2)
MDTRTPLRKKILIISTALGFVLCVGLMIHTKRSIMPPKTHIPTTAKYFPTIGDPYAPINITVFEEPSCSACEEFS
SEVFPLIKKHFVDTGEASLTLVPVCFIRGSMPAAQALLCVYHHDPKRPDPEAYMEYFHRILTYKKTKGSHWATPE
VLAKLAEKIPTHSGREINLKGLIQCINSQRFTEQLKKNNIYGSQIMGGQLATPTAVVGDYLIEDPTFDEIERVIT
QLRHLQAIEEEVR
SEQ ID: 10 CT177 DNA

| | |
|---|---|
| 1 | TCACCGGACC TCCTCTTCTA TCGCTTGTAG ATGACGCAGT TGAGTAATCA CTCTCTCGAT |
| 61 | CTCATCAAAA GTGGGATCTT CAATAAGATA ATCTCCTACG ACTGCAGTAG GTGTTGCAAG |
| 121 | TTGCCCACCC ATGATTTGAG ATCCATAGAT ATTGTTCTTT TTAAGCTGCT CCGTAAATCT |
| 181 | TTGAGAATTT ATGCACTGTA TTAAACCTTT GAGATTAATT TCTCTTCCGG AATGCGTAGG |
| 241 | GATCTTTTCT GCTAATTTTG CAAGCACTTC AGGAGTTGCC CAGTGTGATC CTTTCGTTTT |
| 301 | TTTATATGTG AGAATTCTGT GGAAATATTC CATATATGCT TCTGGATCTG GACGCTTCGG |
| 361 | ATCGTGATGG TAAACGCACA GTAATGCTTG TGCAGCAGGC ATTGAGCCAC GAATAAAACA |
| 421 | TACAGGAACT AAAGTCAGAG AAGCTTCACC AGTGTCAACA AAATGTTTTT TAATCAAAGG |
| 481 | AAATACTTCC GAAGAAAACT CTTCACAGGC AGAACAAGAT GGTTCTTCAA AAACGGTGAT |
| 541 | ATTAATAGGT GCATAAGGAT CCCCTATCGT AGGGAAATAC TTTGCTGTGG TTGGAATATG |
| 601 | CGTCTTTGGT GGCATAATCG AACGCTTAGT GTGTATCATT AATCCTACAC ACAAAACAAA |
| 661 | TCCTAGTGCC GTAGAAATAA TAAGGATCTT CTTTCTCAAG GGAGTTCTCG TATCCAT |

SEQ ID: 11 CT725 polypeptide (184 amino acids; GenBank AAC68320.1)
MKEIYYEIARTESTNTTAKEGLSLWDPYALTVITTREQTAGRGKFGRVWHSTDQDLLASFCFFLSVNNVDSALLF
RIGTEAVMRLGESLGIQEAVMKWPNDVLVQGKKLSGVLCETIPVKTGTCVIIGIGVNGNVGADELLGIDQPATSL
QELIGRPVDMEEQLKRLTKEIKHLIQTLPLWGRE
SEQ ID: 12 CT725 DNA

| | |
|---|---|
| 1 | ATGAAAGAAA TCTATTATGA AATAGCACGT ACGGAATCAA CGAATACGAC AGCAAAAGAG |
| 61 | GGGCTTTCTT TGTGGGATCC CTATGCTCTC ACAGTGATCA CGACCAGAGA ACAAACGGCG |
| 121 | GGAAGAGGGA AATTTGGAAG GGTCTGGCAC TCCACAGATC AAGATCTTTT GGCTTCGTTT |
| 181 | TGTTTCTTTT TAAGTGTGAA TAATGTGGAC AGTGCTTTGT TATTTCGTAT AGGGACAGAA |
| 241 | GCCGTGATGC GTCTCGGGGA ATCGTTAGGC ATTCAAGAAG CTGTCATGAA ATGGCCTAAC |
| 301 | GACGTGTTAG TTCAGGGGAA AAAACTTTCA GGAGTGTTGT GTGAGACCAT CCCTGTTAAG |
| 361 | ACTGGAACGT GTGTCATTAT TGGTATCGGT GTGAATGGTA ATGTGGGTGC TGATGAATTG |
| 421 | CTAGGTATTG ATCAGCCTGC AACGTCTCTC CAGGAATTGA TAGGGAGGCC TGTAGATATG |
| 481 | GAAGAACAGC TTAAGCGGCT CACGAAAGAA ATCAAGCATC TTATCCAGAC GCTACCGTTA |
| 541 | TGGGGGCGAG AATAA |

SEQ ID: 13 CT856 polypeptide (567 amino acids; GenBank AAC68453.1)
MVKVSLSFKHLVPKLVTCLKEGYSFNTLKKDFTAGITAGILAFPLAIAIAIGIGVSPLQGLLASIIGGFLASALG
GSRVLISGPTSSFISILYCIGVKYGEDGLFTITLMAGIFLIIFGLAGLGTFIKYMPYPVVTGLTTGIAVIIFSSQ
IRDFLGLQMGDGVPLDFIGKWAAYWDYLWTWDSKTFAVGLFTLLLMIYFRNYKPRYPGVMISIIIASTLVWILKI
DIPTIGSRYGTLPSSLPGPVFPHISITKMLQLMPDALTISVLSGIETLLAAVVADGMTGWRHQSNCQLIGQGIAN
IGTSLFAGMPVTGSLSRTTASIKCGASTPIAGIIHAICLSFILLLLAPLTIKIPLTCLAAVLILIAWNMSEIHHF
IHLFTAPKKDVVVLLTVFILTVMTTITSAVQVGMMLAAFLFMKQMSDLSDVISTAKYFDESEQPQNDLLFSKNEV
PPFTEIYEINGPFFFGIADRLKNLLNEIEKPPKIFILCMTRVPTIDASAMHALEEFFLECDRQGTLLLLAGVKKT
PLSDLRRYHVDELIGVDHIFPNIKGALLFAKALIKLESKSSQ
SEQ ID: 14 CT856 DNA

| | |
|---|---|
| 1 | CTATTGAGAA GACTTACTCT CTAACTTAAT AAGGGCTTTT GCAAACAATA ACGCACCTTT |
| 61 | AATGTTTGGG AAGATATGGT CTACTCCGAT CAATTCATCT ACATGGTACC TTCTCAAATC |
| 121 | ACTGAGAGGA GTTTTTTTCA CGCCAGCTAA GAGAAGCAAT GTTCCTTGTC GGTCGCATTC |
| 181 | CAAGAAGAAC TCTTCTAGAG CGTGCATGGC AGATGCATCT ATTGTAGGCA CTCGAGTCAT |
| 241 | GCAAAGGATA AATATTTTAG GCGGCTTTTC TATTTCATTT AATAAGTTTT TCAAACGATC |
| 301 | TGCGATGCCA AAGAAAAACG GTCCGTTGAT TTCATAAATT TCCGTAAAAG GTGGTACTTC |
| 361 | ATTTTTGCTA AATAGCAAGT CATTTTGAGG TTGTTCGGAT TCATCAAAAT ATTTTGCTGT |
| 421 | GGAGATAACA TCAGATAGAT CGCTCATTTG TTTCATGAAT AGAAAGGCTG CAAGCATCAT |
| 481 | TCCTACTTGT ACTGCAGAAG TAATCGTAGT CATTACTGTA AGAATGAACA CGGTTAGCAG |
| 541 | GACAACAACG TCTTTTTTAG GAGCTGTGAA TAGATGAATG AAATGGTGAA TTTCACTCAT |
| 601 | ATTCCAAGCA ATTAAAATTA AAACAGCTGC TAGACATGTT AGAGGGATTT TAATAGTTAA |
| 661 | GGGAGCTAGG AGTAGTAGGA TAAAGGAAAG ACAGATGGCA TGGATTATTC CTGCTATAGG |
| 721 | AGTACTAGCG CCGCACTTGA TGCTAGCCGT TGTTCTTGAA AGCGAGCCTG TAACAGGCAT |
| 781 | GCCAGCAAAT AAAGAGGTTC CAATGTTAGC AATTCCTTGG CCAATTAATT GGCAGTTGGA |
| 841 | TTGATGTCTC CACCCAGTCA TTCCATCTGC AACGACAGCT GCTAATAAGG TTTCTATTCC |
| 901 | AGAAAGAACG GAAATAGTTA AAGCATCTGG CATAAGTTGA AGCATTTTAG TAATGCTTAT |
| 961 | GTGTGGGAAA ACTGGACCAG GTAAAGAGCT TGGTAAGGTA CCATAACGGC TACCGATGGT |
| 1021 | AGGGATGTCT ATTTTAAGAA TCCATACTAG AGTCGATGCA ATGATAATAG AAATCATTAC |
| 1081 | GCCGGGATAA CGAGGTTTGT AATTGCGAAA GTAGATCATT AGAAGCAGGG TAAATAAACC |
| 1141 | CACAGCAAAG GTCTTGCTAT CCCAGGTCCA TAGGTAATCC CAATAGGCTG CCCATTTGCC |
| 1201 | GATGAAGTCT AAAGGAACTC CATCTCCCAT TTGAAGCCCA AGAAAATCTC GGATTTGGGA |
| 1261 | AGAAAAAATG ATGACCGCAA TTCCCGTAGT TAGTCCGGTC ACCACAGGAT ACGGCATATA |
| 1321 | TTTAATAAAA GTGCCTAGTC CGGCAAGACC AAAGATAATG AGGAAGATCC CAGCCATCAA |
| 1381 | TGTGATAGTA AACAGTCCGT CTTCGCCATA TTTGACACCG ATACAGTAAA GGATGGAGAT |
| 1441 | AAAGGAACTG GTAGGGCCAG AGATTAATAC ACGACTGCCT CCTAAGGCAG AGGCTAAAAA |
| 1501 | GCCTCCAATA ATTGAGGCCA ATAGTCCTTG TAAAGGAGAC ACTCCAATCC CGATCGCAAT |
| 1561 | AGCAATAGCT AAAGGGAAGG CTAGAATCCC TGCAGTGATC CCTGCGGTAA AGTCTTTTTT |
| 1621 | GAGCGTATTA AAAGAATACC CTTCTTTTAA GCAGGTAACT AATTTAGGGA CAAGATGTTT |
| 1681 | GAAGGATAGG GAAACTTTCA CCAA |

SEQ ID: 15 CT757 polypeptide (336 amino acids; GenBank AAC68352.1)
MLPLTYVVKAFSIGLFFSLFLMKPLISWLKKQGFQDHIHKDHCEKLEELHKDKAYIPTAGGIVFVFASVLAVLLL
FPIQLWSTWFCIGTILLWGALGWCDDQIKNRRRVGHGLSAKHKFLIQNCLAAGVVLPIMFAYKESFLSFHLPFLG
IVSLPHHWWSYLLSFAIATLAIVGTSNSVNLTDGLDGLAAGAMVIACLGMLVVACTNGAPWAFICCVLLATLAGS
CLGFLRYNKSPARVFMGDTGSLFLGAMLGMCAVLLRAEFLLLFMGGIFVLESLSVIVQVGSYKLRKKRVFLCAPL
HHHYEYKGLSEKAVVRNFLIVELICVVVGIIAVFVD
SEQ ID: 16 CT757 DNA

| | |
|---|---|
| 1 | ATGCTGCCCC TAACGTATGT TGTGAAAGCC TTTTCTATTG GCTTGTTTTT TAGCCTTTTT |
| 61 | TTGATGAAAC CTTTGATTTC TTGGTTAAAA AAACAAGGTT TTCAAGATCA TATTCACAAA |
| 121 | GATCACTGCG AAAAATTAGA AGAGTTACAT AAAGACAAAG CATATATCCC TACAGCTGGA |
| 181 | GGGATAGTTT TTGTTTTTGC ATCTGTGTTG GCGGTTCTTT TATTGTTCCC CATACAGCTT |
| 241 | TGGTCTACAT GGTTTTGTAT TGGAACTATT CTATTATGGG GAGCATTAGG ATGGTGCGAT |
| 301 | GATCAGATTA AAAATCGGCG TAGAGTAGGG CATGGGTTGT CTGCTAAACA TAAGTTTCTT |
| 361 | ATACAGAATT GTTTGGCTGC AGGGGTGGTT CTTCCTATTA TGTTCGCATA TAAAGAAAGT |
| 421 | TTTCTTAGTT TTCATCTTCC TTTTCTAGGA ATCGTTTCTT TGCCACATCA TTGGTGGAGC |
| 481 | TATCTACTCA GTTTTGCTAT TGCAACATTG GCTATTGTTG GAACGAGCAA TTCAGTCAAT |
| 541 | CTCACTGATG GATTGGATGG ACTTGCGGCA GGAGCTATGG TGATAGCCTG CTTAGGGATG |
| 601 | CTTGTCGTTG CTTGTACTAA TGGAGCTCCT TGGGCCTTCA TTTGTTGTGT TCTTCTAGCT |
| 661 | ACCTTAGCTG GAAGTTGTCT TGGATTTTTA CGTTACAACA AGTCTCCTGC CCGTGTCTTT |
| 721 | ATGGGAGATA CAGGATCTTT GTTTTTAGGA GCCATGCTCG GTATGTGTGC TGTATTATTA |
| 781 | CGAGCAGAGT TTCTTCTCTT GTTTATGGGA GGGATTTTTG TTCTGGAATC ACTATCTGTG |
| 841 | ATTGTACAAG TCGGAAGTTA TAAATTAAGA AAGAAACGAG TCTTTCTTTG TGCCCCTTTA |
| 901 | CACCATCATT ATGAGTATAA GGGGTTATCA GAAAAGGCTG TAGTGAGGAA TTTCTTAATT |
| 961 | GTCGAGCTTA TTTGTGTAGT AGTTGGGATC ATTGCAGTAT TTGTGGATTA G |

SEQ ID: 17 CT564 polypeptide (289 amino acids; GenBank AAC68166.1)
MATLPEVLSGLGSSYIDYIFQKPADYVWTVFLLLAARILSMLSIIPFLGAKLFFSPIKIGIALSWMGLLLPQVIQ
DSTIVHYQDLDIFYILLIKEILIGVLIGFLFSFPFYAAQSAGSFITNQQGIQGLEGATSLVSIEQTSPHGIFYHY
FVTIVFWLAGGHRIILSVLLQSLEIIPLHAVFPESMMSLRAPMWIAILKMCQLCLIMTIQLSAPAAVAMLMSDLF
LGIINRMAPQVQVIYLLSALKAFMGLLFLTLAWWFIVKQIDYFTLAWFKEIPTMLFGAHPPKVL
SEQ ID: 18 CT564 DNA

| | |
|---|---|
| 1 | ATGGCTACGC TTCCCGAGGT TCTTTCAGGG CTCGGCTCTT CCTATATCGA TTATATATTC |
| 61 | CAAAAGCCAG CCGATTACGT TTGGACTGTC TTTCTTTTGC TAGCGGCACG CATATTATCT |
| 121 | ATGCTGTCGA TCATCCCGTT CTTAGGAGCT AAACTATTCC CGTCACCAAT TAAAATTGGG |
| 181 | ATAGCGCTCT CTTGGATGGG ATTGCTGCTA CCTCAGGTGA TACAAGACTC TACGATCGTC |
| 241 | CACTACCAAG ACCTAGATAT TTTCTATATC CTTCTTATTA AGGAGATTTT GATTGGCGTA |
| 301 | CTCATCGGCT TTCTGTTCTC TTTTCCCTTC TATGCTGCCC AGTCTGCAGG ATCCTTTATT |
| 361 | ACCAACCAGC AAGGGATACA AGGATTAGAA GGTGCTACCT CTCTCGTATC TATAGAACAA |
| 421 | ACTTCTCCTC ACGGGATCTT TTATCATTAT TTTGTGACTA TCGTTTTCTG GCTCGCAGGA |
| 481 | GGACATCGCA TTATCCTTTC TGTTCTTTTA CAATCGCTTG AGATCATCCC TCTTCATGCT |
| 541 | GTTTTCCCTG AGAGCATGAT GTCGCTACGA GCTCCTATGT GGATCGCGAT ATTAAAAATG |
| 601 | TGCCAATTGT GCTTGATTAT GACCATACAG TTGAGCGCTC CAGCAGCGGT GGCTATGCTT |
| 661 | ATGTCAGATT TATTCCTAGG GATCATCAAC CGAATGGCTC CTCAGGTACA AGTCATCTAC |
| 721 | CTACTTTCTG CACTGAAAGC CTTTATGGGA TTGTTATTCC TAACACTGGC TTGGTGGTTC |
| 781 | ATTGTGAAAC AAATTGATTA TTTCACTCTG GCATGGTTCA AAGAAATCCC TACTATGCTC |
| 841 | TTCGGAGCTC ATCCTCCTAA AGTTTTGTGA |

SEQ ID: 19 CT703 polypeptide (490 amino acids; GenBank AAC68298.1)
MRIAILGRPNVGKSSLFNRLCKRSLAIVNSQEGTTRDRLYGEIRAWDSIIHVIDTGGVDQESTDRFQKQIHQQAL
AAAEEASVLLLVVDIRCGITKQDEELAKRLLPLKKPLILVMNKADSQQDLQRIHEFYGLGISDMIATSASHDKHI
DLLLERIRQIAQIPVPSVEEQDAVQEDELPSEEAAISLHAFADETLFENESLSQEEASFLEELVAQTATPAPVDR
PLKVALIGHPNVGKSSIINALLKEERCITDNSPGTTRDNIDVAYTHNNKEYVFIDTAGLRKTKSIKNSVEWMSSS
RTEKAISRTDICLLVIDATQQLSYQDKRILSMIARYKKPHVILVNKWDLMFGVRMEHYVQDLRKMDPYIGQARIL
CISAKQRRNLLQIFSAIDDIYTIATTKLSTSLVNKVLASAMQRHHPQVINGKRLRIYYAIHKTTTPFTFLLFINS
NSLLTKPYELYLKNTLKAAFNLYRVPFDLEYKAKPARKSN
SEQ ID: 20 CT703 DNA

| | |
|---|---|
| 1 | TTAATTTGAT TTTCTTGCAG GTTTTGCTTT GTATTCTAAA TCAAATGGAA CTCTATATAA |
| 61 | ATTAAAAGCT GCTTTTAAAG TGTTTTTTAA ATACAACTCG TAAGGTTTCG TCAGCAGACT |
| 121 | ATTGGAATTG ATAAACAGCA AGAAAGTAAA TGGTGTCGTC GTCTTATGAA TCGCATAGTA |
| 181 | GATGCGTAAA CGTTTGCCAT TAATGACCTG CGGATGGTGT CTTTGCATAG CAGAAGCTAA |
| 241 | TACCTTGTTA ACTAAGGAAG TCGAGAGTTT TGTCGTTGCA ATAGTATAGA TATCATCAAT |
| 301 | AGCAGAAAAG ATTTGTAACA GATTGCGGCG TTGCTTGGCT GAAATACAAA GTATGCGCGC |
| 361 | TTGACCTATA TAGGGATCCA TTTTTCGCAA GTCTTGAACA TAATGTTCCA TGCGAACACC |
| 421 | AAACATTAAG TCCCATTTAT TTACGAGAAT CACATGAGGT TTTTTATATC TCGCAATCAT |
| 481 | AGATAGAATC CGCTTATCTT GATAGGAGAG CTGCTGGGTC GCATCGATCA CTAATAGGCA |
| 541 | AATGTCTGTT CTGGAAATGG CTTTTTCTGT TCGAGAAGAA GACATCCATT CCACAGAGTT |
| 601 | TTTAATGCTC TTAGTTTTTC TTAATCCGGC AGTATCTATA AAGACGTATT CTTTATTGTT |
| 661 | ATGCGTATAG GCAACATCGA TGTTGTCTCG TGTAGTCCCT GGAGAATTAT CCGTTATACA |
| 721 | GCGCTCCTCC TTAAGAAGAG CATTGATAAT GGAGGATTTC CCTACATTGG GATGCCCAAT |
| 781 | CAACGCTACC TTTAACGGGC GGTCTACAGG GGCTGGCGTC GCCGTCTGCG CAACGAGCTC |
| 841 | TTCAAGGAAA GAAGCTTCTT CTTGCGATAG GGATTCATTT TCAAAAAGAG TTTCATCAGC |
| 901 | AAAGGCATGC AAAGATATAG CAGCCTCTTC AGAGGGGAGC TCGTCTTCTT GTACAGCATC |
| 961 | TTGTTCTTCT ACAGAAGGTA CAGGGATCTG CGCGATCTGA CGGATGCGTT CCAAGAGTAA |
| 1021 | ATCAATATGC TTATCATGGC TAGCCGATGT GGCAATCATA TCAGAGATTC CCAATCCATA |
| 1081 | AAATTCATGA ATGCGCTGTA AATCCTGCTG GGAATCCGCT TTATTCATAA CAAGAATCAA |
| 1141 | AGGCTTCTTC AACGGCAGGA GACGCTTAGC CAGCTCTTCA TCTTGTTTGG TGATACCACA |
| 1201 | TCGGATATCT ACTACAAGCA GCAGAACAGA GGCTTCCTCT GCTGCTGCTA AAGCCTGTTG |
| 1261 | ATGAATTTGC TTTTGGAATC GGTCGGTAGA CTCTTGGTCT ACGCCCCCAG TATCGATAAC |
| 1321 | ATGGATAATA GAATCCCAGG CTCGAATTTC TCCATACAAA CGATCTCGCG TAGTTCCTTC |
| 1381 | TTGAGAGTTC ACAATCGCTA AAGAGCGTTT ACATAAGCGG TTGAAGAGAG AAGACTTCCC |
| 1441 | TACATTGGGT CTTCCTAAAA TAGCAATACG CAT |

SEQ ID: 21 P1-ORF7 polypeptide (PGP7-D; 160 amino acids; GenBank
NP_040380.1)
MGSMAFHKSRLFLTFGDASEIWLSTLSYLTRKNYASGINFLVSLEILDLSETLIKAISLDHSESLFKIKS
LDVFNGKVVSEASKQARAACYISFTKFLYRLTKGYIKPAIPLKDFGNTTFFKIRDKIKTESISKQEWTVF
FEALRIVNYRDYLIGKLIVQGIRKLDEILSLRTDDLFFASNQISFRIKKRQNKETKILITFPISLMEELQ
KYTCGRNGRVFVSKIGIPVTTSQVAHNFRLAEFHSAMKIKITPRVLRASALIHLKQIGLKDEEIMRISCL
SSRQSVCSYCSGEEVIPLVQTPTIL
SEQ ID: 22 P1-ORF7 DNA (PGP7-D CALCULATED_MOL_WT = 34705)

| | |
|---|---|
| 7022 | ATGGGCTCG ATGGCTTTCC ATAAAAGTAG ATTGTTTTTA ACTTTTGGGG ACGCGTCGGA |
| 7081 | AATTTGGTTA TCTACTTTAT CTTATCTAAC TAGAAAAAAT TATGCGTCTG GGATTAACTT |
| 7141 | TCTTGTTTCT TTAGAGATTC TGGATTTATC GGAAACCTTG ATAAAGGCTA TTTCTCTTGA |
| 7201 | CCACAGCGAA TCTTTGTTTA AAATCAAGTC TCTAGATGTT TTTAATGGAA AAGTTGTTTC |
| 7261 | AGAGGCATCT AAACAGGCTA GAGCGGCATG CTACATATCT TTCACAAAGT TTTTGTATAG |
| 7321 | ATTGACCAAG GGATATATTA AACCCGCTAT TCCATTGAAA GATTTTGGAA ACACTACATT |
| 7381 | TTTTAAAATC CGAGACAAAA TCAAAACAGA ATCGATTTCT AAGCAGGAAT GGACAGTTTT |
| 7441 | TTTTGAAGCG CTCCGGATAG TGAATTATAG AGACTATTTA ATCGGTAAAT TGATTGTACA |
| 7501 | AG |

SEQ ID: 23 CT067 polypeptide (326 amino acids; GenBank AAC67658.1)
MSFFHTRKYKLILRGLLCLAGCFLMNSCSSSRGNQPADESIYVLSMNRMICDCVSRITGDRVKNIVLIDGAIDPH
SYEMVKGDEDRMAMSQLIFCNGLGLEHSASLRKHLEGNPKVVDLGQRLLNKNCFDLLSEEGFPDPHIWTDMRVWG
AAVKEMAAALIQQFPQYEEDFQKNADQILSEMEELDRWAARSLSTIPEKNRYLVTGHNAFSYFTRRYLSSDAERV
SGEWRSRCISPEGLSPEAQISIRDIMRVVEYISANDVEVVFLEDTLNQDALRKIVSCSKSGQKIRLAKSPLYSDN
VCDNYFSTFQHNVRTITEELGGTVLE
SEQ ID: 24 CT067 DNA

| | |
|---|---|
| 1 | ATGTCTTTTT TTCATACTAG AAAATATAAG CTTATCCTCA GAGGACTCTT GTGTTTAGCA |
| 61 | GGCTGTTTCT TAATGAACAG CTGTTCCTCT AGTCGAGGAA ATCAACCCGC TGATGAAAGC |
| 121 | ATCTATGTCT TGTCTATGAA TCGCATGATT TGTGATTGCG TGTCTCGCAT AACTGGGGAT |
| 181 | CGAGTCAAGA ATATTGTTCT GATTGATGGA GCGATTGATC CTCATTCATA TGAGATGGTG |
| 241 | AAGGGGGATG AAGACCGAAT GGCTATGAGC CAGCTGATTT TTTGCAATGG TTTAGGTTTA |
| 301 | GAGCATTCAG CTAGTTTACG TAAACATTTA GAGGGTAACC CAAAAGTCGT TGATTTAGGT |
| 361 | CAACGTTTGC TTAACAAAAA CTGTTTTGAT CTTCTGAGTG AAGAAGGATT CCCTGACCCA |
| 421 | CATATTTGGA CGGATATGAG AGTATGGGGT GCTGCTGTAA AAGAGATGGC TGCGGCATTA |
| 481 | ATTCAACAAT TTCCTCAATA TGAAGAAGAT TTTCAAAAGA ATGCGGATCA GATCTTATCA |
| 541 | GAGATGGAGG AACTTGATCG TTGGGCAGCG CGTTCTCTCT CTACGATTCC TGAAAAAAAT |
| 601 | CGCTATTTAG TCACAGGCCA CAATGCGTTC AGTTACTTTA CTCGTCGGTA TCTATCCTCT |
| 661 | GATGCGGAGA GAGTGTCTGG GGAGTGGAGA TCGCGTTGCA TTTCTCCAGA AGGGTTGTCT |
| 721 | CCTGAGGCTC AGATTAGTAT CCGAGATATT ATGCGTGTAG TGGAGTATAT CTCTGCAAAC |
| 781 | GATGTAGAAG TTGTCTTTTT AGAGGATACC TTAAATCAAG ATGCTTTGAG AAAGATTGTT |
| 841 | TCTTGCTCTA AGAGCGGACA AAAGATTCGT CTCGCTAAGT CTCCTTTATA TAGCGATAAT |
| 901 | GTCTGTGATA ACTATTTTAG CACGTTCCAG CACAATGTTC GCACAATTAC AGAAGAATTG |
| 961 | GGAGGGACTG TTCTTGAATA G |

SEQ ID: 25 CT037 polypeptide (118 amino acids; GenBank AAC67627.1)
MESFFVLKIPFFLLNGVQDSPCLSLVLFYSFFPFTLNWFATLGGRPTAPRNSVLIQLKLKKILSTTLVIQESPNT
KKAPREYTVRGDFSKLLNFGIIEASEIRKVPMKSALHCTLRED
SEQ ID: 26 CT037 DNA

| | |
|---|---|
| 1 | TTAATCCTCT CTAAGAGTGC AATGCAACGC ACTTTTCATA GGGACTTTTC GTATTTCTGA |
| 61 | GGCCTCAATG ATGCCAAAAT TGAGGAGTTT AGAAAAGTCG CCTCGGACAG TATACTCCCT |
| 121 | TGGAGCTTTT TTAGTATTTG GGCTTTCCTG TATTACGAGA GTGGTCGATA GAATTTTTTT |
| 181 | TAATTTTAGC TGAATTAGAA CGCTATTTCG CGGTGCAGTT GGTCTACCAC CAAGAGTTGC |
| 241 | AAACCAATTG AGGGTGAACG GGAAAAATGA ATAAAAAAGG ACGAGAGAGA GACAGGGACT |
| 301 | ATCTTGAACT CCATTTAGCA GAAAAAAAGG TATTTTCAAA ACAAAAAAAG ACTCCAT |

SEQ ID: 27 CT252 polypeptide (272 amino acids; GenBank AAC67845.1)
MIHWDQSRTLLSFPRVGLHLSWYGILFSLGIFLSSFSGIKLATALCKDREEKKELRTSLENFALGALLAIIIGAR
LAYVLFYGGSFYFENPSEIIKIWKGGLSSHGAVISVVIWAAVFSRLHIRKLPMLSVTYICDLCGAVFGCAALLIR
VGNFMNQEILGTPTSMPWGVIFPNGGGQIPRHPVQLYEGLGYLVLSCILYRLCYRGVIRLGSGYSAAGALIGVAV
IRFCAEFFKTHQGAWLGEENILTIGQWLSIPMIFLGVGIIWIASKKK
SEQ ID: 28 CT252 DNA

| | |
|---|---|
| 1 | TCATTTTTTT TTACTAGCAA TCCAAATGAT TCCAACTCCT AGAAAAATCA TCGGAATAGA |
| 61 | CAACCATTGC CCAATTGTTA ATATGTTTTC TTCGCCAAGC CATGCTCCTT GGTGTGTTTT |
| 121 | GAAAAATTCA GCGCAAAAAC GAATTACTGC TACCCCAATT AAAGCGCCTG CTGCACTATA |
| 181 | GCCAGAACCC AAACGAATAA CACCACGATA GCAAAGCCTG TACAGAATAC AAGAAAGCAC |
| 241 | TAAATAACCA AGGCCTTCGT AAAGCTGAAC AGGATGTCTA GGGATTTGGC CTCCACCATT |
| 301 | CGGAAAAATC ACTCCCCAAG GCATGGATGT AGGGGTTCCT AGAATTTCCT GATTCATAAA |
| 361 | GTTCCCCACG CGAATCAGCA AAGCTGCACA ACCAAACACT GCTCCACAAA GATCGCAAAT |
| 421 | GTAGGTTACT GAAAGCATAG GCAACTTACG AATATGAAGT CGCGAAAATA CAGCTGCCCA |
| 481 | AATCACCACA GAGATCACAG CTCCATGACT AGAAAGCCCT CCTTTCCATA TTTTTATAAT |
| 541 | CTCAGAAGGA TTTTCAAAAT AAAAACTCCC TCCATAGAAA AGAACGTAAG CAAGCCTAGC |
| 601 | TCCAATGATG ATAGCTAAAA GAGCTCCTAA AGCAAAATTT TCCAGACTTG TTCGGAGTTC |
| 661 | TTTTTTCTCC TCCCTGTCTT TACACAATGC TGTTGCCAGC TTGATGCCCG AAAAAGATGA |
| 721 | TAAAAAAATT CCTAGAGAAA ATAAGATTCC GTACCACGAT AAATGAAGCC CAACTCGCGG |
| 781 | GAAAGATAAG AGAGTTCTAG ACTGGTCCCA ATGTATCAC |

SEQ ID: 29 CT064 polypeptide (602 amino acids; GenBank AAC67655.1)
MKPYKIENIRNFSIIAHIDHGKSTIADRLLESTSTIEQREMREQLLDSMDLERERGITIKAHPVTMTYEYEGETY
ELNLIDTPGHVDFSYEVSRSLAACEGALLIVDAAQGVQAQSLANVYLALERDLEIIPVLNKIDLPAAQPEAIKKQ
IEEFIGLDTSNTIACSAKTGQGIPEILESIIRLVPPPKPPQETELKALIFDSHYDPYVGIMVYVRVISGEIKKGD
RITFMATKGSSFEVLGIGAFLPEATLMEGSLRAGQVGYFIANLKKVKDVKIGDTVTTVKHPAKEPLEGFKEIKPV
VFAGIYPIDSSDFDTLKDALGRLQLNDSALTIEQENSHSLGFGFRCGFLGLLHLEIIFERISREFDLDIIATAPS
VIYKVVLKNGKTLFIDNPTAYPDPALIEHMEEPWVHVNIITPQEYLSNIMSLCMDKRGICLKTDMLDQHRLVLSY
ELPLNEIVSDFNDKLKSVTKGYGSFDYRLGDYKKGAIIKLEILINDEAVDAFSCLVHRDKAESKGRSICEKLVDV
IPPQLFKIPIQAAINKKIIARETIRALAKNVTAKCYGGDITRKRKLWDKQKKGKKRMKEFGKVSIPNTAFVEVLK
ME
SEQ ID: 30 CT064 DNA

| | |
|---|---|
| 1 | CTACTCCATT TTAAGGACTT CAACAAACGC CGTGTTCGGA ATGGATACTT TTCCGAATTC |
| 61 | TTTCATTCGT TTCTTCCCTT TTTTCTGTTT GTCCCACAAC TTGCGTTTTC TTGTGATATC |
| 121 | TCCACCATAG CACTTAGCAG TTACATTTTT CGCTAAAGCT CGAATCGTCT CTCTGGCAAT |
| 181 | AATCTTTTTA TTGATGGCCG CCTGAATAGG GATTTTAAAG AGCTGAGGAG GGATAACATC |
| 241 | TACGAGTTTC TCGCAGATGC TTCTGCCTTT TGATTCTGCT TTGTCTCTGT GTACAAGGCA |
| 301 | GGAAAAGGCA TCAACAGCCT CATCATTAAT TAGAATTTCC AGCTTAATGA TAGCACCCTT |
| 361 | TTTATAATCT CCTAACCGGT AATCAAAGGA GCCGTATCCT TTCGTCACAG ATTTGAGTTT |
| 421 | ATCATTGAAA TCAGAAACAA TCTCATTGAG AGGCAGCTCA TATGAAAGCA CCAGTCTGTG |
| 481 | TTGGTCAAGC ATATCTGTTT TTAGACAGAT CCCACGCTTA TCCATACAAA GGCTCATAAT |
| 541 | ATTGCTGAGA TACTCTTGAG GCGTAATGAT ATTAACATGG ACCCAAGGCT CCTCCATGTG |
| 601 | TTCAATAAGA GCTGGGTCAG GATATGCTGT TGGGTTATCA ATAAAAAGGG TTTTACCATT |
| 661 | TTTTAAGACG ACTTTGTAGA TAACGCTAGG AGCTGTAGCA ATAATATCGA GATCAAATTC |
| 721 | TCTAGAGATT CTCTCAAAGA TGATTTCTAA GTGCAGCAGT CCTAAAAATC CACAGCGGAA |
| 781 | CCCAAATCCG AGAGAATGAC TGTTCTCTTG TTCAATCGTA AGAGCTGAGT CGTTTAGCTG |
| 841 | CAACCGGCCT AGAGCATCTT TCAGGGTATC AAAGTCAGAA GAATCTATAG GATAGATACC |
| 901 | AGCAAACACT ACAGGTTTGA TTTCTTTAAA GCCTTCTAAA GGCTCTTTAG CAGGATGTTT |
| 961 | AACAGTAGTG ACTGTATCGC CAATTTTTAC ATCCTTTACT TTTTTTAGGT TGGCAATGAA |
| 1021 | GTATCCCACT TGTCCGGCTC GTAAGGATCC TTCCATGAGA GTAGCTTCCG GTAAGAAAGC |
| 1081 | TCCTATTCCT AAGACCTCAA AAGAGGAGCC TTTGGTTGCC ATGAAGGTAA TGCGATCTCC |
| 1141 | CTTTTTGATT TCTCCACTGA TCACGCGTAC ATAAACCATG ATTCCTACAT AAGGATCGTA |
| 1201 | GTGAGAATCA AAGATCAAAG CTTTAAGTTC TGTTTCCTGT GGAGGTTTTG GTGGGGGAAC |
| 1261 | GAGTCGTATA ATAGACTCTA AAATTTCAGG GATACCCTGA CCTGTTTTCG CTGAGCAAGC |
| 1321 | AATGGTGTTT GAAGTATCTA ATCCGATGAA CTCTTCGATT TGTTTTTTTA TAGCTTCTGG |
| 1381 | TTGAGCAGCA GGTAAGTCTA TTTTATTTAA AACAGGAATG ATTTCTAAAT CTCGTTCTAG |
| 1441 | AGCCAGATAT ACATTAGCTA AGCTTTGAGC TTGAACACCT TGGGCAGCAT CTACTATAAG |
| 1501 | CAGCGCTCCT TCACAAGCTG CTAGTGATCG GGATACTTCA TAAGAGAAAT CTACGTGTCC |
| 1561 | AGGAGTATCT ATTAGATTGA GTTCGTAAGT CTCCCCTTCG TATTCATAGG TCATAGTGAC |
| 1621 | CGGATGCGCT TTGATGGTAA TCCCGCGTTC TCTTTCTAGA TCCATAGAAT CTAAAAGTTG |
| 1681 | TTCGCGCATC TCTCTTTGTT CGATAGTACT AGTACTTTCT AACAAACGAT CTGCGATCGT |
| 1741 | AGATTTCCCG TGGTCGATAT GAGCAATGAT AGAAAAATTA CGAATGTTCT CAATTTTATA |
| 1801 | CGGTTTCAA |

SEQ ID: 31 CT137 polypeptide (281 amino acids; GenBank AAC67728.1)
MFSQQIEESIKAGQVFAFPTDTVYGLGVSFHILDADQRLFALKHRSSQKALSVYVSSLEELEAVAQQSLGASSRK
IIQKFLPGPLTLITKHNNPRFPQKTLGFRIVNHPIVQQIIQKVGPFLATSANLSGFPSAVSADEVKQDFPEEDIV
MISGECSIGLESTVIDPEERIVYRESAISIAEIETVLGAPCANLSKELGFREKIGIHVVKTPADLCSFLLSRPHF
KGVICHQPHPHTFYSVLRQALRSPTQEIIFVYDLCNTEYPILSRFLGVSYDSGYAL
SEQ ID: 32 CT137 DNA

| | |
|---|---|
| 1 | GTGTTTTCGC AACAGATTGA GGAGAGCATT AAGGCGGGGC AAGTTTTTGC CTTCCCTACA |
| 61 | GATACAGTAT ATGGTTTGGG AGTGTCTTTT CATATCCTTG ATGCTGATCA GCGATTATTT |
| 121 | GCTCTTAAGC ACAGATCTTC CCAAAAAGCT CTGTCCGTCT ATGTCTCATC TTTAGAAGAA |
| 181 | TTAGAGGCTG TTGCCCAACA GTCTTTAGGA GCATCTTCGA GAAAGATAAT TCAAAAGTTT |
| 241 | CTTCCTGGGC CTCTTACCTT GATTACAAAA CATAATAATC CGAGATTTCC TCAGAAAACA |
| 301 | TTGGGATTCA GGATTGTTAA TCATCCTATA GTGCAGCAGA TCATTCAAAA AGTAGGGCCG |
| 361 | TTTCTTGCTA CTTCAGCGAA TCTATCCGGC TTTCCTTCTG CAGTTTCTGC TGATGAGGTA |
| 421 | AAACAAGATT TCCCGGAAGA AGATATCGTA ATGATTTCAG GAGAATGTTC TATAGGGTTG |
| 481 | GAGTCTACAG TAATCGATCC TGAGGAGCGA ATTGTTTATC GTGAGAGTGC TATTTCTATT |
| 541 | GCAGAAATAG AAACTGTATT AGGGGCTCCA TGTGCTAATC TGTCTAAGGA ACTAGGGTTT |
| 601 | AGAGAAAAAA TAGGTATCCA TGTTGTAAAA ACCCCCGCAG ATTTATGTAG TTTTCTTTTG |
| 661 | TCTAGACCTC ATTTTAAGGG TGTTATTTGC CATCAGCCTC ATCCTCATAC TTTTTATTCT |
| 721 | GTTCTAAGGC AGGCTTTACG CTCTCCTACA CAAGAAATCA TTTTCGTTTA CGATTTGTGC |
| 781 | AATACAGAAT ATCCAATTCT TTCACGTTTT CTAGGAGTGA GTTATGATAG TGGATATGCA |
| 841 | TTGTGA |

SEQ ID: 33 CT204 polypeptide (471 amino acids; GenBank AAC67796.1)
MNKHKRFLSLVLLTFILLGIWFCPHSDLIDSKAWHLFAIFTTTIIGIIVQPAPMGAIVIMGISLLLVTKTLTLDQ
ALSGFHSPITWLVFLSFSIAKGVIKTGLGERVAYFFVKILGKSPLGLSYGLVLTDFLLAPAIPSLTARAGGILFP
VVMGLSESFGSSVEKGTEKLLGSFLIKVAYQSSVITSAMFLTAMAGNPIISALASHSGVTLTWAIWAKTAILPGI
ISLACMPFVLFKLFPPQITSCEEAVATAKTRLKEMGPLNQGERIILLIFSLLISLWTFGDSIGISATTTTFIGLS
LLILTNILDWQKDVLSNTTAWETFFWFGALIMMASFLSAFGFIHFVGDSVIGSVQGLSWKIGFPILFTVSISLGA
NPMFAALALAFASNLFGGLTHYGSGPAPLYFGSHFVSVQEWWRSGFILSIVNLTIWLGLGSWWWYCLGLIR
SEQ ID: 34 CT204 DNA

| | |
|---|---|
| 1 | ATGAATAAAC ACAAACGCTT CTTATCGCTC GTACTCTTAA CATTTATCCT TCTCGGAATT |
| 61 | TGGTTCTGCC CGCATTCTGA TCTCATCGAC TCCAAAGCGT GGCACTTATT TGCGATATTT |
| 121 | ACTACGACTA TTATCGGAAT CATTGTACAA CCCGCTCCTA TGGGAGCCAT TGTTATCATG |
| 181 | GGCATTTCTC TTCTGCTTGT GACCAAAACA TTAACTCTAG ATCAAGCTTT GTCCGGATTT |
| 241 | CATAGCCCTA TTACTTGGCT TGTATTTCTT TCGTTTTCCA TAGCAAAAGG CGTGATTAAA |
| 301 | ACAGGTCTTG GAGAGCGAGT TGCTTACTTC TTTGTAAAAA TATTGGGTAA AAGTCCTTTA |
| 361 | GGATTGAGCT ATGGCTTAGT TCTTACAGAC TTTTTATTAG CACCGGCAAT CCCTAGTTTG |
| 421 | ACAGCTCGCG CTGGAGGCAT TCTTTTCCCT GTTGTTATGG GATTATCAGA GTCTTTCGGT |
| 481 | AGTTCTGTAG AAAAAGGCAC GGAAAAACTT CTCGGATCTT TTTTAATCAA AGTAGCTTAT |
| 541 | CAAAGCTCTG TAATTACAAG TGCTATGTTT TTAACTGCTA TGGCTGGAAA CCCTATTATT |
| 601 | TCTGCCTTAG CAAGTCATTC TGGAGTAACG TTAACATGGG CAATTTGGGC TAAAACCGCA |
| 661 | ATCCTTCCAG GGATTATTAG CTTAGCCTGT ATGCCTTTTG TACTCTTTAA ACTATTCCCA |
| 721 | CCACAAATAA CTAGCTGTGA AGAAGCTGTA GCAACTGCCA AAACTCGCTT AAAAGAAATG |
| 781 | GGACCTTTAA ATCAAGGCGA ACGCATTATT CTTTTAATCT TTTCTCTTTT AATATCTTTA |
| 841 | TGGACTTTCG GAGATTCCAT CGGCATCTCA GCAACAACCA CAACATTTAT AGGACTATCC |
| 901 | CTACTCATTC TTACGAATAT TCTTGATTGG CAAAAAGATG TTCTTTCTAA CACTACTGCA |
| 961 | TGGGAAACCT TTTTCTGGTT CGGAGCTTTA ATTATGATGG CTTCCTTCCT AAGCGCTTTT |
| 1021 | GGGTTTATTC ATTTTGTAGG AGATTCTGTT ATTGGGAGCG TTCAAGGTCT ATCTTGGAAA |
| 1081 | ATAGGGTTCC CTATACTCTT TCTTATTTAT TTCTACTCTC ACTATCTATT TGCGAGTAAT |
| 1141 | ACAGCACATA TTGCAGCCAT GTACCCTATC TTTCTTACAG TATCCATCTC CTTAGGCGCG |
| 1201 | AATCCTATGT TTGCTGCCTT AGCCTTAGCT TTTGCTAGTA ATTTATTCGG AGGACTCACA |
| 1261 | CACTACGGAT CTGGTCCAGC TCCGTTATAC TTTGGATCCC ATTTCGTCTC CGTGCAAGAA |
| 1321 | TGGTGGCGCT CTGGCTTTAT TCTTAGCATA GTCAATCTAA CCATTTGGTT GGGATTAGGA |
| 1381 | AGTTGGTGGT GGTACTGTTT AGGATTAATT CGCTAA |

SEQ ID: 35 CT634 polypeptide (465 amino acids; GenBank AAC68238.1)
MKIVVSRGLDLSLKGAPKESGFCGKVDPTYVSVDLRPFAPLPLGVKVTPEDQVTAGSPLAEYKLFSGVFITSPVD
GEVVEIRRGNKRALLEIVIKKKPGISQTKFSYDLQSLTQKDLLEVFKKEGLFALFKQRPFDIPALPTQSPRDVFI
NLADNRPFTPSVEKHLSLFSSKEDGYYIFVVGVQAIAKLFGLKPHIISTDRLTLPTQDLVSIAHLHTIDGPFPSG
SPSTHIHHIARIRNERDVVFTISFQEVLSIGHLFLKGFVLGQQIVALAGSALPPSQRKYLITAKGASFSDLLPKD
IFSSDEITLISGDPLTGRLCKKEENPCLGMRDHTITLLPNPKTRESFSFLRLGWNKLTVTRTYLSGFFKRKRVFM
DMDTNMHGEKRPIIDAEIYERVSAIPVPVALIIKALETQNFEEACRLGLLEVAPEDFALPTFIDPSKTEMFSIVK
ESLLRYAKENVVTSS
SEQ ID: 36 CT634 DNA

| | |
|---|---|
| 1 | TTACGAGGAG GTTACCACAT TCTCTTTTGC GTAGCGTAAA AGAGATTCTT TGACGATAGA |
| 61 | GAACATCTCG GTCTTAGAAG GATCTATGAA TGTGGGGAGA GCAAAATCTT CTGGAGCAAC |
| 121 | TTCTAAGAGC CCTAGGCGAC ACGCTTCTTC AAAGTTTTGT GTTTCCAAAG CTTTAATAAT |
| 181 | AAGAGCTACA GGAACCGGGA TTGCTGAAAC ACGCTCATAG ATTTCAGCAT CAATAATGGG |
| 241 | CCGTTTTTCT CCATGCATGT TAGTATCCAT ATCCATGAAG ACCCGTTTTC TCTTGAAAAA |
| 301 | ACCAGATAGA TAGGTTCGTG TGACTGTAAG TTTATTCCAA CCTAAGCGCA AGAAACTGAA |
| 361 | AGATTCACGA GTTTTAGGAT TAGGAAGAAG TGTTATGGTA TGGTCTCTCA TACCTAAACA |
| 421 | AGGATTTTCT TCTTTTTTAC ATAATCTTCC TGTAAGAGGA TCTCCAGAAA TAAGGGTAAT |
| 481 | CTCATCGGAA GAGAAAATGT CTTTAGGAAG AAGATCAGAG AAACTAGCGC CTTTCGCAGT |
| 541 | AATGAGATAT TTTCTTTGAG AAGGAGGAAG AGCTGATCCT GCTAAGGCAA CGATTTGTTG |
| 601 | TCCTAAAACA AAGCCTTTTA AAAATAGATG CCCTATAGAT AACACCTCTT GGAAGCTAAT |
| 661 | AGTAAACACA ACATCTCTTT CGTTTCGAAT ACGAGCGATG TGATGAATGT GCGTTGAAGG |
| 721 | AGATCCTGAT GGGAAGGGGC CATCTATTGT GTGTAAGTGG GCTATGGATA CGAGATCCTG |
| 781 | GGTTGGGAGA GTTAGTCTGT CTGTAGAAAT GATATGAGGC TTCAGTCCAA ATAGTTTTGC |
| 841 | TATTGCCTGA ACTCCCACAA CAAAAATGTA ATAACCATCT TCTTTTGAAG AAAAAAGACT |
| 901 | GAGATGTTTT TCCACAGAAG GGGTGAAAGG GCGATTATCC GCTAAGTTAA TAAAAACATC |
| 961 | TCGAGGAGAT TGTGTTGGAA GAGCTGGGAT ATCAAAAGGT CTTTGTTTGA AAAGAGCGAA |
| 1021 | AAGACCTTCC TTTTTAAAAA CTTCTAAAAG ATCTTTTTGA GTCAAAGATT GAAGATCATA |
| 1081 | AGAAAACTTA GTTTGAGAAA TACCAGGCTT CTTCTTGATG ACGATCTCTA AAAGAGCACG |
| 1141 | TTTATTTCCT CTACGGATCT CTACAACCTC TCCATCAACA GGAGAGGTAA TAAACACTCC |
| 1201 | TGAAAAAAGC TTGTACTCAG CCAGGGGAGA ACCAGCAGTA ACTTGGTCTT CTGGAGTAAC |
| 1261 | CTTTACCCCT AAAGGAAGGG GAGCGAAAGG CCTCAAATCC ACGGAAACAT AGGTGGGGTC |
| 1321 | CACCTTACCG CAAAAACCCG ATTCCTTCGG AGCTCCCTTT AAAGACAGAT CTAATCCGCG |
| 1381 | AGAAACAACT ATTTTCAT |

SEQ ID: 37 CT635 polypeptide (144 amino acids; GenBank AAC68239.1)
MKNNSAQKIIDSIKQILSIYKIDFEPSFGATLTDDNDLDYQMLIEKTQEKIQELDKRSQEILQQTGMTREQMEVF
ANNPDNFSPEEWRALENIRSSCNEYKKETEELIKEVTNDIGHSSHKSPTPKKTKSSSQKKSKKKNWIPL
SEQ ID: 38 CT635 DNA

| | |
|---|---|
| 1 | TTATAAGGGA ATCCAATTTT TTTTCTTACT TTTTTTCTGA GAGGAGGATT TTGTCTTTTT |
| 61 | TGGCGTTGGA GATTTGTGGG ATGAGTGACC AATATCATTG GTTACTTCTT TGATAAGCTC |
| 121 | TTCAGTTTCT TTTTTGTATT CATTGCAAGA GGAACGAATG TTTTCTAGAG CTCGCCACTC |
| 181 | TTCAGGAGAA AAGTTATCTG GATTATTAGC AAAGACTTCC ATTTGTTCGC GAGTCATTCC |
| 241 | CGTCTGTTGG AGAATTTCCT GCGATCTTTT GTCTAATTCT TGGATTTTTT CCTGTGTTTT |
| 301 | TTCGATCAGC ATTTGGTAGT CCAGATCGTT GTCGTCAGTA AGAGTTGCTC CAAAGGAGGG |
| 361 | TTCGAAGTCT ATTTTATAAA TAGAGAGAAT TTGTTTTATA GAATCTATAA TTTTTTGAGC |
| 421 | GGAATTATTT TTCAT |

SEQ ID: 39 CT366 polypeptide (440 amino acids; GenBank AAC67962.1)
MPTFDTTKQIFLCGLPSVGKTSFGQHLSQFLSLPFFDTDHLLSDRFHGDSPKTIYQRYGEEGFCREEFLALTSVP
VIPSIVALGGCTPIIEPSYAHILGRNSALLVLLELPIATLCQRLQHRSIPERLAHAPSLEDTLSQRLDKLRSLTS
NAFSLRAETSSEAVMRDCQSFCLRFLSTKESSYA
SEQ ID: 40 CT366 DNA

| | |
|---|---|
| 1 | ATGGTCTCTT CGAACCAAGA CCTTCTTATT TCTCCCTCAA TTCCTTATGG AGAAATTGCT |
| 61 | GTTCCTCCGT CAAAATCACA TTCTCTACGC GCGATCCTTT TTGCCTCCTT ATCCAAAGGG |
| 121 | ACCTCTATCA TAGAAAACTG TCTCTTCTCT CCCGATTCCC AAGCTATGCT TACAGCCTGT |
| 181 | GAGAAAATGG GAGCTCACGT TAGAAGAATA GGAGACTCCT TACATATCCA GGGGAATCCC |
| 241 | GATCCCCATC ACTGTCACCC ACGCTATTTC CATATGGGGA ATTCTGGTAT CGCCCTTCGA |
| 301 | TTCCTAACCG CCCTTTCTAC TTTATCCCCC ACCCCCACTT TGATCACAGG ATCCCACACA |
| 361 | CTCAAACGAC GTCCTATAGC GCCTCTTCTA TCAAGCTTAA AACAGCTTGG TGCGCACATT |
| 421 | CGCCAAAAAA CATCTTCTTC TATTCCCTTT ACCATCCATG GTCCATTATC CCCTGGCCAT |
| 481 | GTTACTATCT CTGGACAAGA TTCCCAATAC GCATCAGCAT TAGCAATCAC TGCAGCTTTA |
| 541 | GCTCCATATC CCCTTTCTTT TTCTATCGAA AATCTTAAGG AACGTCCTTG GTTTGATCTG |
| 601 | ACCTTAGATT GGCTACACTC TTTAAACATC TCTTTCTTAA GAGACCAAGA TTCTTTAACT |
| 661 | TTCCCCGGAG GACAATCATT AGAAAGTTTT TCTTATTCTG TGCCTGGAGA CTATAGTTCT |
| 721 | GCTGCTTTTT TAGCTTCCTT TGGTCTACTC TCTTCTTCTT CTAAACCAAC TATTCTCCGT |
| 781 | AATCTTTCTT CTCAAGATTC TCAAGGGGAC AAGCTTCTCT TCTCTTTGTT AAAACAACTT |
| 841 | GGAGCCCATA TTCTTATTGG AAAACATCAT ATCGAAATGC ACCCCTCTTC TTTCTCCGGA |
| 901 | GGTGAAATTG ATATGGATCC ATTCATAGAT GCATTACCCA TCCTTGCTGT CCTCTGCTGC |
| 961 | TTTGCAAAAA ATCCATCGCG CTTGTATAAT GCGTTGGGAG CAAAGGACAA AGAAAGCAAT |
| 1021 | CGCATTGAAG CCATTGCCCA TGAATTGCAA AAAATGGGTG GTTCTGTCCA CCCTACTCGT |
| 1081 | GACGGTCTAT ATATAGAGCC CTCGCGGTTA CATGGTGCGG TTGTTGATTC TCATAATGAT |
| 1141 | CACCGTATTG CTATGGCTCT CGCTGTAGCT GGAGTTCATG CCTCGTCCGG ACAAACCCTC |
| 1201 | CTCTGTAACA CACAGTGTAT AAATAAGAGT TTTCCATATT TCGTGATTGC AGCGCAGACA |
| 1261 | CTACATGCCA ACGTTCGACA CTACCAAGCA GATTTTCCTT TGCGGTCTTC CTTCTGTAGG |
| 1321 | TAA |

SEQ ID: 41 CT140 polypeptide (228 amino acids; GenBank AAC67731.1)
MLNETLFVLQILVVIGFGAFFAARNLIMLAAWASLLSIIMNIFVLKQIVLFGFEVTAADVYVIGLFSCLNCAREF
WGKESTRKVIFVSWCSTLSFLILTQLH
LHLKPSPGDISQLHYEALFAPSLRIISASVITTMIVQFVDFKVFGWLKKHSQGRVFGLRSACSVALSQSIDTVIF
SFLGLYGLVANLPDVMMFSLLSKGTALLLASPCVALAKVFYNRLNKEEAHF
SEQ ID: 42 CT140 DNA

| | |
|---|---|
| 1 | ATGTTAAACG AGACATTATT TGTATTGCAA ATCCTTGTAG TTATTGGGTT CGGAGCTTTT |
| 61 | TTTGCTGCGC GTAATCTAAT TATGTTAGCG GCATGGGCCT CATTGCTTTC CATTATCATG |
| 121 | AACATTTTTG TATTAAAGCA AATCGTGTTA TTCGGATTCG AAGTAACTGC AGCGGATGTT |
| 181 | TACGTGATAG GGCTGTTTTC TTGCTTGAAT TGTGCGAGAG AATTCTGGGG GAAGGAGTCT |
| 241 | ACAAGAAAAG TGATTTTTGT TTCTTGGTGC AGCACGCTTT CTTTTCTAAT CCTGACACAA |
| 301 | CTCCATCTCC ATCTTAAGCC TTCTCCAGGA GATATCAGCC AACTGCACTA TGAAGCTCTA |
| 361 | TTCGCCCCTT CTCTTCGGAT TATTTCAGCA TCAGTGATCA CAACGATGAT TGTGCAGTTT |
| 421 | GTTGATTTTA AGGTGTTTGG TTGGCTGAAA AAACATTCGC AAGGACGGGT CTTTGGATTG |
| 481 | CGTTCCGCAT GCTCCGTTGC GCTTTCTCAA AGCATAGACA CCGTAATTTT TTCTTTTCTA |
| 541 | GGTTTGTATG GACTCGTTGC TAACTTACCA GATGTCATGA TGTTTTCTTT GTTATCCAAA |
| 601 | GGGACGGCTC TTTTGTTAGC TTCTCCTTGT GTGGCTCTAG CCAAGGTTTT TTATAATCGC |
| 661 | TTGAATAAAG AAGAAGCACA CTTTTAA |

SEQ ID: 43 CT142 polypeptide (285 amino acids; GenBank AAC67733.1)
MSDSDKIINDCRFDFNTTIHGDLLASNLTTEGDVTVKSISAKESFSVKRNVDVNENDIIVNGFTGAAGYDLTTQG
KISINLNGNRLSNVKRPEKDSQPVPANYIRTPEYYFCSLQDGARIEWKRGQKLPLIGPSRLVYQSSRIDEFIRFV
SFEEDKTKNQVKINLSGTTGLQMLAKGVYIINVGVGKRWGWNNGYGGDYCLAVPLGKEYSESSTFSRGGYYASTA
VGTAIHIRKESTNPDGPFSSSDTELMKTLLEVRYKGGDYVDKSALSTLYFGVLVYPEIGG
SEQ ID: 44 CT142 DNA

| | |
|---|---|
| 1 | ATGAGTGATT CTGACAAAAT TATTAATGAT TGTCGGTTCG ACTTTAATAC AACTATTCAT |
| 61 | GGAGATCTTT TAGCTTCAAA TCTGACTACG GAAGGGGACG TTACGGTAAA GAGTATTTCC |
| 121 | GCAAAAGAAT CCTTTTCTGT GAAAAGAAAT GTTGATGTGA ATGAGAACGA CATCATTGTT |
| 181 | AACGGTTTTA CCGGTGCCGC AGGATATGAT CTGACAACTC AAGGCAAAAT TTCAATCAAT |
| 241 | CTCAACGGTA ATCGACTTAG TAATGTCAAA CGCCCGGAGA AAGACTCCCA ACCAGTTCCT |
| 301 | GCTAACTATA TTCGTACTCC TGAATACTAT TTCTGCTCAT TGCAAGATGG AGCAAGAATC |
| 361 | GAATGGAAAC GGGGGCAGAA GCTTCCTCTA ATCGGGCCTT CGCGCTTGGT GTATCAATCG |
| 421 | TCTCGTATTG ATGAGTTCAT TCGTTTTGTA TCGTTTGAAG AAGATAAAAC TAAGAATCAG |
| 481 | GTGAAAATAA ATCTCTCAGG GACTACAGGC CTGCAAATGC TTGCGAAAGG TGTGTACATT |
| 541 | ATCAACGTAG GAGTTGGGAA GCGATGGGGG TGGAATAATG GATATGGAGG AGATTACTGT |
| 601 | TTAGCGGTCC CTTTAGGAAA GGAATACAGT GAGAGCTCTA CATTTAGTAG AGGAGGATAC |
| 661 | TATGCTTCTA CTGCTGTAGG AACAGCAATT CATATCAGAA AAGAGAGCAC AAATCCTGAC |
| 721 | GGACCTTTTT CTTCTTCAGA TACAGAACTT ATGAAGACAC TTTTAGAGGT GCGTTACAAG |
| 781 | GGCGGAGACT ATGTGGACAA GTCCGCCTTG TCCACTTTAT ATTTTGGAGT GCTCGTATAC |
| 841 | CCAGAGATAG GAGGATAA |

SEQ ID: 45 CT242 polypeptide (173 amino acids; GenBank AAC67835.1)
MKKFLLLSLMSLSSLPTFAANSTGTIGIVNLRRCLEESALGKKESAEFEKMKNQFSNSMGKMEEELSSIYSKLQD
DDYMEGLSETAAAELRKKFEDLSAEYNTAQGQYYQILNQSNLKRMQKIMEEVKKASETVRIQEGLSVLLNEDIVL
SIDSSADKTDAVIKVLDDSFQNN
SEQ ID: 46 CT242 DNA

| | |
|---|---|
| 1 | ATGAAAAAGT TCTTATTACT TAGCTTAATG TCTTTGTCAT CTCTACCTAC ATTTGCAGCT |
| 61 | AATTCTACAG GCACAATTGG AATCGTTAAT TTACGTCGCT GCCTAGAAGA GTCTGCTCTT |
| 121 | GGGAAAAAAG AATCTGCTGA ATTCGAAAAG ATGAAAAACC AATTCTCTAA CAGCATGGGG |
| 181 | AAGATGGAGG AAGAACTGTC TTCTATCTAT TCCAAGCTCC AAGACGACGA TTACATGGAA |
| 241 | GGTCTATCCG AGACCGCAGC TGCCGAATTA AGAAAAAAAT TCGAAGATCT ATCTGCAGAA |
| 301 | TACAACACAG CTCAAGGGCA GTATTACCAA ATATTAAACC AAAGTAATCT CAAGCGCATG |
| 361 | CAAAAGATTA TGGAAGAAGT GAAAAAAGCT TCTGAAACTG TGCGTATTCA AGAAGGCTTG |
| 421 | TCAGTCCTTC TTAACGAAGA TATTGTCTTA TCTATCGATA GTTCGGCAGA TAAAACCGAT |
| 481 | GCTGTTATTA AAGTTCTTGA TGATTCTTTT CAAAATAATT AA |

SEQ ID: 47 CT843 polypeptide (89 amino acids; GenBank AAC68440.2)
MSLDKGTKEEITKKFQLHEKDTGSADVQIAILTEHITELKEHLKRSPKDQNSRLALLKLVGQRRKLLEYLNSTDT
ERYKNLIARLNLRK
SEQ ID: 48 CT843 DNA

| | |
|---|---|
| 1 | CTATTTTCTC AAATTGAGGC GAGCAATTAA ATTTTTATAT CTTTCAGTAT CAGTAGAATT |
| 61 | TAAGTACTCT AGGAGCTTTC TTCTCTGCCC TACTAATTTT AGCAAAGCTA GACGAGAATT |
| 121 | TTGATCTTTA GGAGATCTTT TAAGGTGCTC CTTGAGTTCC GTTATGTGCT CAGTCAGAAT |
| 181 | AGCAATCTGC ACATCTGCCG AACCTGTGTC TTTTTCATGA AGTTGAAATT TTTTAGTAAT |
| 241 | TTCTTCTTTA GTGCCCTTAT CCAAAGACAT |

SEQ ID: 49 CT328 polypeptide (274 amino acids; GenBank AAC67921.1)
MFTDKETHRKPFPTWAHLLHSEPSKQFVFGNWKMNKTLTEAQTFLKSFISSDILSNPQIITGIIPPFTLLSACQQ
AVSDSPIFLGAQTTHEADSGAFTGEISAPMLKDIGVDFVLIGHSERRHIFHEQNPVLAEKAAAAIHSGMIPVLCI
GETLEEQESGATQDILLNQLTTGLSKLPEQASFILAYEPVWAIGTGKVAHPDLVQETHAFCRKTIASLFSKDIAE
RTPILYGGSVKADNARSLSLCPDVNGLLVGGASLSSENFLSIIQQIDIP
SEQ ID: 50 CT328 DNA

| | |
|---|---|
| 1 | ATGTTTACAG ACAAAGAAAC TCACAGAAAA CCATTTCCAA CTTGGGCCCA CCTTCTCCAC |
| 61 | TCTGAGCCAT CAAAGCAATT TGTTTTCGGT AATTGGAAAA TGAACAAAAC ACTTACTGAA |
| 121 | GCTCAGACCT TTTTAAAAAG TTTCATCTCT AGTGACATTC TGTCTAATCC CCAAATCATT |
| 181 | ACAGGAATCA TTCCTCCTTT CACACTGCTG TCAGCTTGTC AACAAGCTGT AAGCGATTCC |
| 241 | CCCATCTTTC TTGGAGCCCA AACCACTCAT GAAGCTGACT CAGGAGCTTT TACTGGTGAG |
| 301 | ATTTCAGCCC CAATGCTCAA AGATATCGGA GTCGATTTTG TTCTCATCGG ACATTCCGAA |
| 361 | AGACGTCATA TCTTTCATGA ACAAAATCCT GTACTTGCTG AAAAAGCTGC TGCAGCTATC |
| 421 | CATAGTGGAA TGATTCCAGT TCTGTGTATT GGAGAAACTC TAGAAGAACA AGAATCTGGA |
| 481 | GCAACTCAAG ATATTCTTTT AAATCAACTG ACTACAGGAT TATCTAAACT CCCTGAGCAA |
| 541 | GCCTCTTTCA TTCTAGCTTA TGAACCAGTC TGGGCTATAG GCACCGGAAA AGTAGCTCAT |
| 601 | CCTGATCTAG TTCAGGAAAC CCATGCTTTC TGTAGAAAAA CGATTGCTTC TCTCTTTTCC |
| 661 | AAAGATATTG CGGAACGCAC CCCCATTCTT TACGGAGGAT CTGTGAAAGC CGATAATGCT |
| 721 | CGCTCACTTT CCCTCTGCCC TGATGTTAAT GGTCTTTTAG TTGGAGGAGC CTCTTTATCT |
| 781 | TCAGAGAATT TCTTATCCAT TATACAACAA ATCGATATCC CATAA |

SEQ ID: 51 CT188 polypeptide (203 amino acids; GenBank AAC67780.1)
MFIVVEGGEGAGKTQFIQALSKRLIEEGREIVTTREPGGCSLGDSVRGLLLDPEQKISPYAELLLFLAARAQHIQ
EKIIPALKSGKTVISDRFHDSTIVYQGIAGGLGESFVTNLCYHVVGDKPFLPDITFLLDIPAREGLLRKARQKHL
DKFEQKPQIFHRSVREGFLALAEKAPDRYKVLDALLPTEASVDQALLQIRALI
SEQ ID: 52 CT188 DNA

| | |
|---|---|
| 1 | CTATATCAAT GCACGAATCT GTAAGAGAGC TTGGTCAACA GAAGCCTCTG TTGGCAAGAG |
| 61 | GGCATCTAAA ACCTTGTACC TATCTGGAGC TTTTTCTGCT AAAGCAAGAA ATCCTTCTCT |
| 121 | GACAGACCGG TGGAAAATTT GTGGTTTTTG CTCAAATTTA TCCAGATGTT TCTGACGAGC |
| 181 | CTTTCGTAGT AATCCTTCTC TTGCTGGGAT ATCCAATAAG AATGTGATGT CTGGCAAGAA |
| 241 | CGGCTTATCT CCCACAACAT GATAACATAA GTTCGTAACA AAACTCTCCC CTAAGCCTCC |
| 301 | AGCAATTCCT TGATATACAA TAGTAGAATC GTGAAAACGA TCGCTTATAA CCGTCTTCCC |
| 361 | AGACTTAAGA GCAGGTATGA TCTTTTCCTG AATGTGTTGT GCACGAGCTG CTAAAAACAA |
| 421 | CAACAATTCT GCATATGGAG ATATTTTTTG TTCTGGATCC AGAAGAAGGC CTCGAACACT |
| 481 | GTCTCCAAGA GAGCATCCCC CTGGCTCTCT CGTAGTGACA ATTTCTCTGC CTTCTTCTAT |
| 541 | TAAACGCTTA GAAAGTGCTT GTATAAACTG AGTTTTCCCA GCACCTTCTC CGCCTTCTAC |
| 601 | TACAATAAAC AC |

SEQ ID: 53 CT578 polypeptide (487 amino acids; GenBank AAC68180.1)
MSLSSSSSSDSSNLKNVLSQVIASTPQGVPNADKLTDNQVKQVQQTRQNRDDLSMESDVAVAGTAGKDRAASASQ
IEGQELIEQQGLAAGKETASADATSLTQSASKGASSQQCIEDTSKSLELSSLSSLSSVDATHLQEIQSIVSSAMG
ATNELSLTNLETPGLPKPSTTPRQEVMEISLALAKAITALGESTQAALENFQSTQSQSANMNKMSLESQGLKIDK
EREEFKKMQEIQQKSGTNSTMDTVNKVMIGVTVAITVISVVSALFTCGLGLIGTAAAGATAAAAGATAAATTATS
VATTVATQVTMQAVVQVVKQAIIQAVKQAIVQAIKQGIKQGIKQAIKQAVKAAVKTLAKNVGKIFSAGKNAVSKS
FPKLSKVINTLGSKWVTLGVGALTAVPQLVSGITSLQLSDMQKELAQIQKEVGALTAQSEMMKAFTLFWQQASKI
AAKQTESPSETQQQAAKTGAQIAKALSAISGALAAAA
SEQ ID: 54 CT578 DNA

| | |
|---|---|
| 1 | ATGTCCCTTT CATCTTCTTC GTCTTCCGAT AGTAGCAACC TTAAGAATGT CTTGTCGCAA |
| 61 | GTCATAGCTT CGACTCCTCA AGGCGTTCCT AATGCAGATA AATTAACCGA CAATCAGGTT |
| 121 | AAGCAAGTTC AACAGACGAG ACAAAATCGC GATGACCTAA GCATGGAAAG CGATGTCGCT |
| 181 | GTTGCCGGAA CTGCTGGAAA AGATCGCGCA GCTTCTGCTT CTCAAATAGA AGGACAAGAA |
| 241 | CTTATAGAGC AGCAAGGATT AGCTGCAGGG AAAGAAACTG CATCTGCCGA TGCGACATCC |
| 301 | CTAACCCAAA GCGCATCTAA AGGAGCTAGC TCGCAACAAT GCATAGAAGA TACTAGCAAA |
| 361 | TCTTTAGAGC TATCTTCTTT AAGTTCGTTG TCATCTGTAG ATGCCACGCA TCTACAAGAA |
| 421 | ATTCAAAGCA TCGTATCCTC TGCTATGGGT GCTACTAACG AGCTTTCCTT GACGAACTTA |
| 481 | GAAACTCCAG GACTACCCAA ACCTTCAACG ACACCTCGTC AAGAAGTAAT GGAAATTAGC |
| 541 | CTTGCATTAG CAAAAGCAAT TACCGCTCTT GGAGAGTCAA CGCAAGCAGC ATTGGAGAAC |
| 601 | TTCCAAAGTA CGCAGTCGCA ATCTGCGAAC ATGAACAAAA TGTCTCTAGA ATCTCAAGGC |
| 661 | CTTAAAATTG ATAAAGAGCG TGAAGAGTTC AAAAAAATGC AAGAGATCCA GCAAAAGTCT |
| 721 | GGAACCAACT CTACCATGGA TACCGTTAAC AAAGTGATGA TTGGGGTTAC CGTGGCTATT |
| 781 | ACTGTGATCT CTGTAGTATC CGCATTATTC ACTTGCGGTC TTGGCTTGAT CGGAACTGCT |
| 841 | GCTGCAGGAG CCACAGCAGC CGCGGCTGGA GCTACAGCAG CAGCAACGAC AGCAACTTCT |
| 901 | GTAGCTACAA CAGTCGCTAC ACAAGTGACT ATGCAAGCAG TCGTGCAAGT GGTTAAACAA |
| 961 | GCTATTATTC AAGCTGTTAA ACAGGCTATC GTCCAAGCTA TTAAACAAGG GATTAAACAA |
| 1021 | GGGATCAAAC AAGCCATTAA GCAAGCTGTT AAGGCGGCTG TGAAAACCCT TGCTAAAAAC |
| 1081 | GTGGGTAAAA TTTTCAGCGC AGGGAAAAAT GCTGTTAGCA AATCGTTCCC TAAACTCTCC |
| 1141 | AAAGTTATCA ACACTTTGGG AAGTAAATGG GTAACCTTAG GAGTAGGAGC TCTTACAGCA |
| 1201 | GTTCCTCAAC TCGTATCCGG GATTACTAGT CTGCAGCTGT CAGACATGCA GAAAGAACTG |
| 1261 | GCCCAAATTC AAAAAGAAGT CGGAGCTCTC ACAGCTCAAT CTGAAATGAT GAAAGCTTTC |
| 1321 | ACATTGTTCT GGCAACAAGC AAGTAAAATT GCAGCTAAAC AAACAGAAAG CCCTAGTGAA |
| 1381 | ACGCAACAGC AGGCGGCCAA AACCGGAGCT CAGATAGCGA AAGCTTTGTC CGCAATAAGT |
| 1441 | GGCGCCTTAG CCGCCGCAGC TTAA |

SEQ ID: 55 CT724 polypeptide (174 amino acids)
MLFWGIFSLCLGGLFGGYCRLRYTAKALLLSWRQLLRLALKKREVLQEIAALQTFPLLRLEEEIAFLKQGSFYSL
KEFLKASDADGVTFYEMERFFTLRLKQTLASLQESLHQEAVQHLMEELLAYENAFSFEAFAFEKAAETYATLHGH
PVIQFSGKLFRFPQISFPPLDEAI
SEQ ID: 56 CT724 DNA
ATGCTTTTTTGGGGCATTTTTAGTTTGTGCTTAGGAGGGTTATTCGGGGGTTATTGTCGC
TTGCGCTATACAGCAAAGGCTCTTTTGTTATCCTGGCGACAACTCCTTCGGCTTGCCTTA
AAAAAAAGAGAGGTTTTACAAGAGATCGCAGCGTTGCAAACATTCCCTCTCCTTCGTTTA
GAAGAGGAGATAGCCTTTTTAAAGCAAGGCTCCTTCTATTCTTTGAAAGAATTTCTTAAA
GCTAGTGATGCGGATGGAGTTACTTTCTATGAGATGGAACGATTTTTTACTCTCCGATTG
AAACAGACATTAGCATCGTTGCAAGAAAGTTTGCATCAAGAGGCTGTCCAGCATTTAATG
GAAGAACTACTTGCGTATGAGAATGCGTTTTCTTTTGAGGCCTTTGCTTTCGAAAAAGCC
GCGGAAACCTATGCGACTCTTCACGGTCATCCGGTAATCCAATTTTCTGGGAAACTTTTT
CGTTTTCCGCAAATCTCCTTTCCGCCTTTAGATGAAGCGATA
SEQ ID: 57 CT722 polypeptide (226 amino acids; GenBank AAC68317.1)
MTLLILLRHGQSVWNQKNLFTGWVDIPLSQQGIQEAIAAGESIKHLPIDCIFTSTLVRSLITALLAMTNHSSQKV
PYIVHEERPDMSRIHSQKEMEQMIPLFQSSALNERMYGELQGKNKQEVAAQFGEEQVKLWRRSYRIAPPQGESLF
DTGQRTLPYFQERIFPLLQQGKNIFISAHGNSLRSLIMDLEKLSEEQVLSLELPTGQPIVYEWTGQKFTKHAPSL
G
SEQ ID: 58 CT722 DNA

| | |
|---|---|
| 1 | TTAACCAAGA GAAGGAGCGT GTTTCGTGAA TTTTTGTCCC GTCCATTCGT ATACAATAGG |
| 61 | CTGTCCTGTT GGCAACTCCA AAGAGAGTAC TTGTTCTTCA GATAATTTTT CTAGGTCCAT |
| 121 | AATTAAGGAG CGCAAAGAAT TCCCGTGAGC AGAGATAAAA ATATTTTTCC CTTGCTGAAG |
| 181 | GAGAGGGAAA ATTCTCTCTT GAAAATAGGG GAGGGTTCGT TGCCCTGTAT CGAAAAGACT |
| 241 | TTCGCCCTGA GGAGGGGCAA TGCGGTAGCT TCGGCGCCAC AGTTTTACCT GTTCTTCTCC |
| 301 | GAATTGAGCA GCGACTTCTT GTTTATTTTT TCCTTGAAGT TCTCCGTACA TGCGTTCATT |
| 361 | GAGAGCGCTA GATTGAAAAA GAGGGATCAT CTGCTCCATT TCTTTTTGAC TATGAATCCG |
| 421 | GCTCATGTCG GGGCGCTCTT CATGAACGAT ATAAGGAACT TTTTGAGAGC TGTGGTTAGT |
| 481 | CATTGCTAAC AGGGCTGTTA TCAAACTTCT AACCAAGGTG GAAGTGAAGA TGCAATCAAT |
| 541 | AGGAAGATGT TTAATAGATT CTCCAGCGGC AATAGCCTCT TGAATTCCTT GTTGGCTAAG |
| 601 | AGGGATGTCT ACCCAGCCTG TAAACAGATT TTTTTGATTC CATACGGATT GGCCATGGCG |
| 661 | TAGCAAGATA AGAAGCGTCA T |

SEQ ID: 59 CT732 polypeptide (157 amino acids; GenBank AAC68327.1)
MKPLKGCPVAKDVRVAIVGSCFNSPIADRLVAGAQETFFDFGGDPSSLTIVRVPGAFEIPCAIKKLLSTSGQFHA
VVACGVLIQGETSHYEHIADSVAAGVSRLSLDFCLPITFSVITAPNMEAAWERAGIKGPNLGASGMKTALEMASL
FSLIGKE
SEQ ID: 60 CT732 DNA

| | |
|---|---|
| 1 | ATGAAACCGT TGAAAGGATG TCCTGTCGCT AAGGATGTGC GTGTAGCTAT TGTTGGGTCA |
| 61 | TGTTTCAATT CTCCTATCGC TGATAGGCTT GTTGCTGGGG CGCAAGAAAC CTTTTTCGAT |
| 121 | TTCGGAGGAG ATCCTTCTTC TTTAACAATT GTCCGAGTCC CTGGGGCGTT TGAGATTCCT |
| 181 | TGTGCGATTA AGAAATTACT TTCCACCTCA GGACAGTTTC ATGCTGTGGT TGCTTGCGGA |
| 241 | GTGTTGATTC AGGGCGAGAC ATCGCATTAT GAACATATAG CAGATAGTGT GGCTGCAGGT |
| 301 | GTTAGTCGCC TATCCTTAGA CTTCTGTCTT CCTATTACAT TTTCCGTGAT TACTGCTCCT |
| 361 | AATATGGAAG CGGCTTGGGA GCGTGCGGGT ATCAAAGGGC CCAATTTAGG CGCTTCAGGC |
| 421 | ATGAAAACAG CTTTAGAAAT GGCATCATTA TTCTCTCTGA TAGGGAAGGA ATAA |

SEQ ID: 61 CT788 polypeptide (166 amino acids; GenBank AAC68383.1)
MNSGMFPFTFFLLYICLGMLTAYLANKKNRNLIGWFLAGMFFGIFAIIFLLILPPLPSSTQDNRSMDQQDSEEFL
LQNTLEDSEIISIPDTMNQIAIDTEKWFYLNKDYTNVGPISIVQLTAFLKECKHSPEKGIDPQELWVWKKGMPNW
EKVKNIPELSGTVKDE
SEQ ID: 62 CT788 DNA
ATGAACTCCGGAATGTTCCCATTCACCTTTTTTTTACTGTACATCTGTCTGGGAATGCTTACGGCGTACCTAGCT
AATAAAAAAAATCGCAATCTAATAGGCTGGTTTTTGGCAGGAATGTTTTTTGGTATTTTTGCCATTATCTTCCTA
TTAATTCTCCCTCCTCTTCCTTCTTCTACACAAGATAATCGTTCCATGGACCAGCAAGATTCCGAAGAATTCCTT
TTACAGAATACTTTAGAGGACTCAGAAATTATTTCCATCCCAGATACAATGAATCAAATTGCGATTGATACAGAA
AAGTGGTTCTACTTAAATAAAGACTATACTAATGTCGGTCCTATTTCCATCGTACAGCTGACCGCATTCTTAAAA
GAATGCAAACACTCTCCTGAAAAAGGGATCGATCCCCAAGAATTATGGGTATGGAAGAAAGGAATGCCTAACTGG
GAAAAGGTGAAGAATATACCGGAACTTTCAGGAACAGTAAAAGACGAGTAA
SEQ ID: 63 CT476 polypeptide (321 amino acids; GenBank AAC68076.1)
MKRLFFICALALSPLAYGAVQKDPMLMKETFRNNYGIIVSKQEWNKRGCDGSITRVFKDGTTTLEVYAQGALHGE
VTRTFPHSTTLAVIETYDQGRLLSKKTFFPNALPAKEEVYHEDGSFSLTRWPDNNNSDTITDPCFVEKTYGGRVL
EGHYTSFNGKYSSTILNGEGVRSTFSSDSILLTEESFNDGVMVKKTTFYSTREPETVTHYVNGYPHGVRFTYLPG
GIPNTIEEWRYGHQDGLTILFKNGCKIAEVPFVRGAKNGIELRYNEQENIAEEISWQHNILHGVRKIHAAGVCKS
EWYYKGKPVSQIKFERLSAAR
SEQ ID: 64 CT476 DNA
ATGAAGCGTTTATTTTTTATCTGCGCCCTCGCCCTTTCTCCTCTAGCATATGGAGCTGTTCAAAAGGATCCTATG
TTAATGAAGGAGACTTTCCGTAATAACTACGGGATCATTGTCTCTAAGCAAGAATGGAACAAACGTGGATGCGAT
GGCTCCATCACTAGAGTATTCAAAGATGGAACTACAACCTTAGAAGTTTATGCGCAAGGTGCTTTACATGGGGAA
GTCACACGAACGTTTCCTCACTCTACTACCCTGGCCGTTATAGAAACTTATGATCAGGGAAGGCTTCTTTCTAAG
AAGACCTTCTTCCCAAATGCTTTGCCTGCTAAAGAAGAAGTTTACCACGAAGATGGGTCTTTCTCCCTAACACGT
TGGCCTGACAATAACAACTCTGACACAATCACAGACCCCTGCTTTGTAGAAAAAACTTATGGGGGAAGAGTATTG
GAAGGTCATTACACCTCTTTTAATGGAAAATACTCTTCAACAATCCTTAACGGCGAGGGAGTTCGCTCTACTTTT
TCTTCGGATAGTATCTTGTTGACAGAAGAGTCGTTTAATGATGGCGTAATGGTCAAAAAAACGACATTTTACTCG
ACTCGAGAACCCGAAACCGTCACTCATTATGTCAATGGGTACCCTCACGGAGTTCGGTTTACCTATCTTCCTGGT
GGGATTCCAAATACGATTGAAGAATGGCGATATGGACATCAAGACGGCCTTACAATCTTATTTAAAAATGGTTGT
AAGATTGCTGAAGTCCCATTTGTACGCGGAGCAAAAAATGGAATCGAACTCCGATACAATGAACAAGAGAATATC
GCTGAAGAGATTTCTTGGCAGCACAACATCTTGCATGGAGTCCGTAAAATCCATGCGGCGGGGGTATGCAAATCC
GAATGGTATTACAAAGGCAAACCTGTCTCGCAAATCAAGTTTGAACGACTCAGCGCTGCCAGATAA
SEQ ID: 65 p6 polypeptide (pGP4-D; 102 amino acids; GenBank AAA91572.1)
MQNKRKVRDDFIKIVKDVKKDFPELDLKIRVNKEKVTFLNSPLELYHKSVSLILGLLQQIENSLGLFPDSPVLEK
LEDNSLKLKKALIMLILSRKDMFSKAE
SEQ ID: 66 p6 DNA
ATGCAAAATAAAAGAAAAGTGAGGGACGATTTTATTAAAATTGTTAAAGATGTGAAAAAAGATTTCCCCGAATTA
GACCTAAAAATACGAGTAAACAAGGAAAAAGTAACTTTCTTAAATTCTCCCTTAGAACTCTACCATAAAAGTGTC
TCACTAATTCTAGGACTGCTTCAACAAATAGAAAACTCTTTAGGATTATTCCCAGACTCTCCTGTTCTTGAAAAA
TTAGAGGATAACAGTTTAAAGCTAAAAAAGGCTTTGATTATGCTTATCTTGTCTAGAAAAGACATGTTTTCCAAG
GCTGAA
SEQ ID: 67 CT310 polypeptide (208 amino acids; GenBank AAC67903.1)
MADLSAQDKLKQICDALREETLKPAEEEAGSIVHNAREQAKRIVEEAKEEAQRIIRSAEETADQTLKKGEAALVQ
AGKRSLENLKQAVETKIFRESLGEWLDHVATDPEVSAKLVQALVQAVDAQGISGNLSAYIGKHVSARAVNEALGK
EITSKLKEKGVSVGNFSGGAQLKVEERNWVLDMSSEVLLDLLTRFLQKDFREMIFQSC
SEQ ID: 68 CT310 DNA
ATGGCAGATCTCAGCGCTCAAGATAAATTAAAGCAAATATGTGATGCTTTGCGAGAGGAAACTTTAAAACCAGCT
GAAGAGGAAGCTGGTTCTATTGTTCATAATGCAAGAGAGCAAGCAAAACGTATTGTTGAGGAGGCCAAGGAAGAG
GCGCAAAGGATTATTCGTTCTGCGGAAGAGACAGCTGACCAAACTCTGAAAAAAGGAGAGGCGGCTTTGGTACAG
GCAGGAAAGCGTTCTTTGGAAAACTTGAAGCAGGCAGTAGAAACGAAGATCTTCAGAGAGTCTTTGGGTGAATGG
TTAGATCATGTGGCTACAGATCCAGAAGTCAGCGCTAAGCTCGTGCAAGCTTTAGTGCAGGCAGTTGATGCACAA
GGGATTTCTGGGAATCTTTCTGCCTATATAGGGAAACACGTGTCAGCTCGAGCTGTCAATGAGGCTTTAGGGAAA
GAGATAACTTCTAAGCTTAAAGAGAAAGGGGTATCTGTTGGCAATTTTTCTGGAGGTGCTCAGTTAAAAGTTGAA
GAGCGCAATTGGGTTTTAGATATGAGCTCAGAGGTTTTGCTAGATTTATTGACTAGATTTTTACAGAAAGATTTT
CGGGAAATGATCTTTCAGTCTTGCTAA
SEQ ID: 69 CT638 polypeptide (255 amino acids; GenBank AAC68242.1)
MNTLGPYHKRVRFITYLFVAFGIIVSWNLPRSAYESIQDTFVRVCSKFLPFRQGSDSLALVEETQCFLLKEKIRL
LEERILSMEEAKQSPPLFSEILSSYFQSPIMGRVIFRDPAHWGSSCWINIGKRQGVKKNSPVVCGKVVVGLVDFV
GEAQSRVRFITDVGIKPSVMAVRGEIQTWVVKDQLRTLARNVANLPASAFADSDKQEALHLLQALEDSLSLSEQN
DFALRGIVCGRGDPIWKPEASILSGTILVL
SEQ ID: 70 CT638 DNA
ATGAATACCCTCGGTCCGTATCATAAACGCGTTCGGTTCATTACGTATCTTTTTGTTGCCTTCGGGATTATTGTG
AGTTGGAATCTTCCTCGAAGTGCTTACGAGTCTATCCAGGATACATTCGTTCGGGTGTGTTCCAAATTTCTTCCA
TTTCGGCAAGGGTCTGATTCTCTGGCCCTTGTTGAAGAAACTCAATGCTTTTTATTGAAAGAAAAAATTCGTTTA
TTGGAAGAGCGTATTCTTTCTATGGAAGAGGCAAAACAGTCTCCGCCTTTGTTTTCAGAAATTCTATCCTCGTAT
TTTCAATCTCCCATTATGGGAAGAGTTATCTTTCGAGATCCAGCACACTGGGGTAGTTCTTGTTGGATTAATATA
GGAAAGCGACAGGGCGTTAAAAAGAATTCTCCTGTTGTTTGCGGTAAGGTTGTTGTGGGGTTGGTGGATTTTGTT
GGTGAAGCGCAGTCTCGTGTACGATTCATCACCGATGTGGGTATCAAACCTTCTGTTATGGCGGTTCGTGGTGAA
ATTCAAACTTGGGTTGTGAAAGATCAGCTACGTACATTAGCTAGGAACGTCGCTAATCTTCCGGCATCTGCTTTT
GCAGATAGTGATAAACAGGAAGCTTTACATCTCTTGCAGGCTCTAGAGGATTCTTTATCTCTATCAGAACAAAAT
GATTTTGCTCTTCGTGGAATTGTTTGTGGTCGTGGGGATCCTATTTGGAAACCGGAGGCTTCTATACTTAGCGGT
ACGATTTTGGTTTTGTAG
SEQ ID: 71 CT172 polypeptide (163 amino acids; GenBank AAC67763.1)
MNYHNTFVKTSMFFLAKRLVQLNKNPFLLKKFSETTVLFIFERQLKMWEGYSIDENNYISDYNMEFGRPLLQKLA
NPVCKALLQKQLEAEQAMTLSNQVTVGDIVLMRSPIFEKSVLLETLINEIIYQESLFLFKKPENVQCPKMSFEHG
AHEILLKIFLTVS
SEQ ID: 72 CT172 DNA
ATGAATTATCACAACACTTTTGTAAAAACCAGCATGTTTTTCTTGGCAAAAAGACTAGTTCAGTTAAATAAAAAT
CCTTTCTTACTCAAAAAGTTTTCAGAAACAACGGTTCTTTTTATATTCGAACGACAACTTAAAATGTGGGAAGGT
TATTCTATAGACGAGAATAATTATATATCTGATTATAACATGGAATTTGGGCGACCTTTATTACAAAAACTAGCA
AATCCAGTATGCAAAGCTTTGTTGCAAAAACAGCTCGAAGCCGAGCAAGCAATGACGTTATCCAATCAAGTCACT
GTTGGAGATATAGTGCTTATGCGTTCTCCAATTTTCGAAAAATCTGTATTATTAGAAACTTTAATCAACGAGATT
ATTTATCAAGAATCGTTATTTTTGTTTAAGAAACCAGAAAATGTTCAATGTCCGAAGATGAGTTTCGAGCACGGT
GCACACGAAATCTTGTTGAAGATCTTTTTGACGGTCTCA
SEQ ID: 73 CT443 polypeptide (553 amino acids; GenBank AAC68042.1)
MRIGDPMNKLIRRAVTIFAVTSVASLFASGVLETSMAESLSTNVISLADTKAKDNTSHKSKKARKNHSKETPVDR
KEVAPVHESKATGPKQDSCFGRMYTVKVNDDRNVEITQAVPEYATVGSPYPIEITATGKRDCVDVIITQQLPCEA
EFVRSDPATTPTADGKLVWKIDRLGQGEKSKITVWVKPLKEGCCFTAATVCACPEIRSVTKCGQPAICVKQEGPE
NACLRCPVVYKINIVNQGTATARNVVVENPVPDGYAHSSGQRVLTFTLGDMQPGEHRTITVEFCPLKRGRATNIA
TVSYCGGHKNTASVTTVINEPCVQVSIAGADWSYVCKPVEYVISVSNPGDLVLRDVVVEDTLSPGVTVLEAAGAQ
ISCNKVVWTVKELNPGESLQYKVLVRAQTPGQFTNNVVVKSCSDCGTCTSCAEATTYWKGVAATHMCVVDTCDPV
CVGENTVYRICVTNRGSAEDTNVSLMLKFSKELQPVSFSGPTKGTITGNTVVFDSLPRLGSKETVEFSVTLKAVS
AGDARGEAILSSDTLTVPVSDTENTHIY
SEQ ID: 74 CT443 DNA
ATGCGAATAGGAGATCCTATGAACAAACTCATCAGACGAGCAGTGACGATCTTCGCGGTGACTAGTGTGGCGAGT
TTATTTGCTAGCGGGGTGTTAGAGACCTCTATGGCAGAGTCTCTCTCTACAAACGTTATTAGCTTAGCTGACACC
AAAGCGAAAGACAACACTTCTCATAAAAGCAAAAAAGCAAGAAAAAACCACAGCAAAGAGACTCCCGTAGACCGT
AAAGAGGTTGCTCCGGTTCATGAGTCTAAAGCTACAGGACCTAAACAGGATTCTTGCTTTGGCAGAATGTATACA
GTCAAAGTTAATGATGATCGCAATGTTGAAATCACACAAGCTGTTCCTGAATATGCTACGGTAGGATCTCCCTAT
CCTATTGAAATTACTGCTACAGGTAAAAGGGATTGTGTTGATGTTATCATTACTCAGCAATTACCATGTGAAGCA
GAGTTCGTACGCAGTGATCCAGCGACAACTCCTACTGCTGATGGTAAGCTAGTTTGGAAAATTGACCGCTTAGGA
CAAGGCGAAAAGAGTAAAATTACTGTATGGGTAAAACCTCTTAAAGAAGGTTGCTGCTTTACAGCTGCAACAGTA
TGCGCTTGTCCAGAGATCCGTTCGGTTACAAAATGTGGACAACCTGCTATCTGTGTTAAACAAGAAGGCCCAGAG
AATGCTTGTTTGCGTTGCCCAGTAGTTTACAAAATTAATATAGTGAACCAAGGAACAGCAACAGCTCGTAACGTT
GTTGTTGAAAATCCTGTTCCAGATGGTTACGCTCATTCTTCTGGACAGCGTGTACTGACGTTTACTCTTGGAGAT
ATGCAACCTGGAGAGCACAGAACAATTACTGTAGAGTTTTGTCCGCTTAAACGTGGTCGTGCTACCAATATAGCA
ACGGTTTCTTACTGTGGAGGACATAAAAATACAGCAAGCGTAACAACTGTGATCAACGAGCCTTGCGTACAAGTA
AGTATTGCAGGAGCAGATTGGTCTTATGTTTGTAAGCCTGTAGAATATGTGATCTCCGTTTCCAATCCTGGAGAT
CTTGTGTTGCGAGATGTCGTCGTTGAAGACACTCTTTCTCCCGGAGTCACAGTTCTTGAAGCTGCAGGAGCTCAA
ATTTCTTGTAATAAAGTAGTTTGGACTGTGAAAGAACTGAATCCTGGAGAGTCTCTACAGTATAAAGTTCTAGTA
AGAGCACAAACTCCTGGACAATTCACAAATAATGTTGTTGTGAAGAGCTGCTCTGACTGTGGTACTTGTACTTCT
TGCGCAGAAGCGACAACTTACTGGAAAGGAGTTGCTGCTACTCATATGTGCGTAGTAGATACTTGTGACCCTGTT
TGTGTAGGAGAAAATACTGTTTACCGTATTTGTGTCACCAACAGAGGTTCTGCAGAAGATACAAATGTTTCTTTA
ATGCTTAAATTCTCTAAAGAACTGCAACCTGTATCCTTCTCTGGACCAACTAAAGGAACGATTACAGGCAATACA
GTAGTATTCGATTCGTTACCTAGATTAGGTTCTAAAGAAACTGTAGAGTTTTCTGTAACATTGAAAGCAGTATCA
GCTGGAGATGCTCGTGGGGAAGCGATTCTTTCTTCCGATACATTGACTGTTCCAGTTTCTGATACAGAGAATACA
CACATCTATTAA
SEQ ID: 75 CT525 polypeptide (284 amino acids; GenBank AAC68126.1)
MFKKFKPVTPGTRQLILPSFDELTTQGELKGSSSRRSVRPNKKLSFFKKSSGGRDNLGHISCRHRGGGVRRHYRV
IDFKRNKDGIEAKVASVEYDPNRSAYIALLNYVDGEKRYILAPKGIKRGDRVISGEGSPFKTGCCMTLKSIPLGL
SVHNVEMRPGSGGKLVRSAGLSAQIIAKTAGYVTLKMPSGEFRMLNEMCRATVGEVSNADHNLCVDGKAGRRRWK
GIRPTVRGTAMNPVDHPHGGGEGRHNGYISQTPWGKVTKGLKTRDKRKSNKWIVKDRRK
SEQ ID: 76 CT525 DNA
ATGTTTAAAAAGTTTAAGCCAGTAACTCCCGGGACGAGACAGTTAATTCTGCCTTCTTTTGATGAGCTTACTACT
CAAGGAGAGTTAAAGGGATCTAGTTCTAGAAGAAGTGTTCGTCCAAATAAAAAGCTTTCTTTTTTCAAAAAGAGC
TCTGGAGGACGAGATAATTTAGGACATATTTCCTGCCGCCATCGTGGAGGAGGAGTAAGACGTCATTATAGAGTG
ATCGACTTCAAACGTAATAAAGACGGTATTGAAGCGAAGGTTGCTTCTGTGGAGTATGATCCAAACCGTTCTGCT
TATATTGCTCTATTGAATTATGTAGATGGAGAAAAGCGTTATATTCTAGCTCCTAAAGGAATTAAGCGAGGCGAT
CGTGTGATTTCTGGAGAAGGAAGTCCTTTCAAAACTGGATGCTGCATGACTCTTAAGAGCATCCCTCTGGGACTT
TCTGTTCATAACGTGGAGATGAGACCTGGCTCCGGGGGTAAATTAGTCCGTTCTGCAGGACTTTCAGCCCAGATC
ATCGCTAAAACAGCTGGATACGTCACTTTGAAGATGCCTTCTGGCGAATTTCGTATGTTGAATGAAATGTGCCGA
GCTACTGTCGGAGAGGTCTCCAATGCAGATCACAATCTGTGTGTAGACGGTAAAGCTGGGCGTCGTCGATGGAAA
GGAATTCGGCCAACAGTTCGAGGAACAGCTATGAACCCTGTTGATCACCCACACGGAGGTGGTGAAGGGCGTCAT
AACGGATACATTTCCCAGACCCCTTGGGGTAAAGTCACGAAAGGATTGAAAACTCGTGATAAGCGTAAGAGTAAT
AAGTGGATAGTTAAGGATAGAAGGAAATAG
SEQ ID: 77 CT606 polypeptide (209 amino acids; GenBank AAC68209.1)
MKILIASSHGYKVRETKVFLKKLGEFDIFSLVDYPSYHPPKETGETPEENAIQKGLFAAQTFRCWTIADDSMLII
PALGGLPGKLSASFAGEQANDKDHRKKLLENMRLLENTIDRSAYFECCVALISPFGKIFKAHASCEGTIAFEERG
SSGFGYDPLFVKHDYKQTYAELPEAIKNQVSHRAKALVKLQPYVETVLANHLLAGKESL
SEQ ID: 78 CT606 DNA
ATGAAAATTCTTATAGCCAGTTCTCATGGATATAAGGTGCGCGAAACCAAGGTTTTTCTAAAAAAACTAGGAGAG
TTTGATATCTTCTCGCTTGTAGACTACCCATCCTACCACCCCCCTAAGGAAACTGGCGAAACCCCAGAAGAAAAT
GCTATTCAGAAAGGCTTATTTGCAGCTCAAACCTTTCGTTGTTGGACTATTGCTGATGATTCTATGCTTATCATT
CCAGCTTTAGGTGGACTCCCAGGAAAATTATCCGCTTCTTTTGCTGGAGAACAGGCAAACGATAAAGATCATCGC
AAAAAACTTCTTGAGAACATGCGTCTTTTAGAAAATACTATCGACCGATCGGCTTATTTTGAATGCTGCGTCGCT
TTAATTTCTCCTTTTGGAAAGATCTTCAAAGCTCACGCCTCTTGCGAAGGAACGATTGCGTTTGAGGAACGCGGT
TCCTCAGGGTTTGGATATGATCCTTTGTTTGTAAAACATGACTACAAGCAAACTTATGCCGAATTACCAGAGGCA
ATTAAAAACCAAGTTTCTCACAGAGCAAAAGCATTAGTCAAATTACAGCCCTATGTGGAAACGGTTCTCGCAAAT
CACTTACTCGCGGGGAAAGAGAGTCTCTAA
SEQ ID: 79 CT648 polypeptide (424 amino acids; GenBank AAC68825.1)
MCVSRSLRWCLCFLLLCGWVDAGVYDKLRLTGINIIDRNGLSETICSKEKLQKYTKIDFLSPQPYQKVMRTYKNA
AGESVACLTTYYPNGQIRQYLECLNNRAFGRYREWHSNGKIHIQAEVIGGIADLHPSAEAGWLFDGTTYAHDSEG
RLEAVIHYEKGLLEGISLYYHANGNVWKECPYHKGVAHGDFLVFTEEGSLLKKQTFCKGQLSGCVLRYEPGSQSL
LSEEEYKQGKLRSGKYYDPLTKEEIACVVNGKGKQVIYGKYAIIETRQIVHGVPHGEVLLFDEHGKSLLQAYSLI
NGQKEGEEVFFYPGGEGRKMLLTWSQGILQGAVKTWYPNGALESSKELVQNKKTGILMLYYPEGQVMATEEYVDD
LLIKGEYFRPNDRYPYAKVEKGCGTAVFFSATGGLLKKVLYEDGKPVIH
SEQ ID: 80 CT648 DNA
ATGTGTGTAAGTAGAAGCTTAAGATGGTGTTTATGTTTTCTTTTGCTGTGCGGATGGGTGGACGCTGGGGTTTAT
GATAAGCTCCGACTGACAGGCATTAACATTATCGATAGGAATGGTCTTTCTGAGACGATCTGTTCTAAAGAAAAA
TTACAAAAGTATACGAAAATCGATTTTCTCTCTCCTCAGCCTTACCAAAAAGTCATGCGTACATACAAAAACGCA
GCAGGCGAGTCGGTTGCTTGTTTAACGACGTACTATCCGAATGGCCAAATCCGACAATATCTCGAGTGTTTAAAT
AATCGTGCTTTTGGACGTTATCGTGAGTGGCATAGTAATGGCAAAATTCATATCCAGGCAGAAGTTATTGGAGGG
ATAGCAGATTTGCATCCTTCCGCAGAAGCCGGATGGTTGTTCGATGGAACAACGTATGCACATGATAGCGAAGGG
CGGTTAGAAGCTGTTATTCATTATGAAAAAGGCTTGCTGGAAGGGATTTCGCTGTATTACCACGCGAATGGGAAT
GTATGGAAGGAATGTCCTTACCATAAAGGTGTTGCTCATGGAGACTTTTTGGTCTTCACCGAAGAAGGAAGTTTG
TTAAAGAAACAAACTTTTTGTAAAGGGCAGTTGTCTGGATGTGTATTACGCTACGAGCCAGGTTCACAGTCATTG
TTGTCAGAAGAAGAATATAAACAAGGGAAACTGCGCAGTGGTAAATATTACGATCCTCTTACTAAGGAAGAAATC
GCGTGCGTAGTGAATGGCAAAGGTAAACAAGTAATTTATGGGAAATATGCGATTATAGAGACCCGACAGATTGTA
CATGGCGTTCCTCACGGGGAAGTCTTGTTATTTGATGAACATGGTAAATCTCTGTTGCAAGCATATTCTCTAATC
AATGGGCAGAAAGAGGGAGAAGAAGTATTTTTCTATCCAGGCGGAGAAGGTAGAAAAATGTTATTAACATGGTCC
CAAGGTATTCTACAAGGAGCTGTGAAAACTTGGTACCCAAATGGCGCTTTGGAAAGTAGCAAAGAACTTGTTCAA
AATAAAAAGACTGGGATTCTCATGCTATACTATCCCGAAGGACAAGTGATGGCTACCGAGGAATATGTAGACGAT
CTTCTCATAAAAGGAGAATATTTCCGGCCGAACGACCGATATCCATATGCTAAAGTGGAAAAAGGTTGTGGGACA
GCGGTCTTTTTCAGTGCTACAGGAGGACTGTTAAAGAAAGTCCTCTATGAAGATGGGAAGCCTGTTATTCATTAG
SEQ ID: 81 CT870 polypeptide (1034 amino acids; GenBank AAC68468.1)
MIKRTSLSFACLSFFYLSTISILQANETDTLQFRRFTFSDREIQFVLDPASLITAQNIVLSNLQSNGTGACTISG
NTQTQIFSNSVNTTADSGGAFDMVTTSFTASDNANLLFCNNYCTHNKGGGAIRSGGPIRFLNNQDVLFYNNISAG
AKYVGTGDHNEKNRGGALYATTITLTGNRTLAFINNMSGDCGGAISADTQISITDTVKGILFENNHTLNHIPYTQ
AENMARGGAICSRRDLCSISNNSGPIVFNYNQGGKGGAISATRCVIDNNKERIIFSNNSSLGWSQSSSASNGGAI
QTTQGFTLRNNKGSIYFDSNTATHAGGAINCGYIDIRDNGPVYFLNNSAAWGAAFNLSKPRSATNYIHTGTGDIV
FNNNVVFTLDGNLLGKRKLFHINNNEITPYTLSLGAKKDTRIYFYDLFQWERVKENTSNNPPSPTSRNTITVNPE
TEFSGAVVFSYNQMSSDIRTLMGKEHNYIKEAPTTLKFGTLAIEDDAELEIFNIPFTQNPTSLLALGSGATLTVG
KHGKLNITNLGVILPIILKEGKSPPCIRVNPQDMTQNTGTGQTPSSTSSISTPMIIFNGRLSIVDENYESVYDSM
DLSRGKAEQLILSIETTNDGQLDSNWQSSLNTSLLSPPHYGYQGLWTPNWITTTYTITLNNNSSAPTSATSIAEQ
KKTSETFTPSNTTTASIPNIKASAGSGSGSASNSGEVTITKHTLVVNWAPVGYIVDPIRRGDLIANSLVHSGRNM
TMGLRSLLPDNSWFALQGAATTLFTKQQKRLSYHGYSSASKGYTVSSQASGAHGHKFLLSFSQSSDKMKEKETNN
RLSSRYYLSALCFEHPMFDRIALIGAAACNYGTHNMRSFYGTKKSSKGKFHSTTLGASLRCELRDSMPLRSIMLT
PFAQALFSRTEPASIRESGDLARLFTLEQAHTAVVSPIGIKGAYSSDTWPTLSWEMELAYQPTLYWKRPLLNTLL
IQNNGSWVTTNTPLAKHSFYGRGSHSLKFSHLKLFANYQAEVATSTVSHYINAGGALVF
SEQ ID: 82 CT870 DNA
ATGATTAAAAGAACTTCTCTATCCTTTGCTTGCCTCAGTTTTTTTTATCTTTCAACTATATCCATTTTGCAAGCT
AATGAAACGGATACGCTACAGTTCCGGCGATTTACTTTTTCGGATAGAGAGATTCAGTTCGTCCTAGATCCCGCC
TCTTTAATTACCGCCCAAAACATCGTTTTATCTAATTTACAGTCAAACGGAACCGGAGCCTGTACCATTTCAGGC
AATACGCAAACTCAAATCTTTTCTAATTCCGTTAACACCACCGCAGATTCTGGTGGAGCCTTTGATATGGTTACT
ACCTCATTCACGGCCTCTGATAATGCTAATCTACTCTTCTGCAACAACTACTGCACACATAATAAAGGCGGAGGA
GCTATTCGTTCCGGAGGACCTATTCGATTCTTAAATAATCAAGACGTGCTTTTTTATAATAACATATCGGCAGGG
GCTAAATATGTTGGAACAGGAGATCACAACGAAAAAAATAGGGGCGGTGCGCTTTATGCAACTACTATCACTTTG
ACAGGGAATCGAACTCTTGCCTTTATTAACAATATGTCTGGAGACTGCGGTGGAGCCATCTCTGCTGACACTCAA
ATATCAATAACTGATACCGTTAAAGGAATTTTATTTGAAAACAATCACACGCTCAATCATATACCGTACACGCAA
GCTGAAAATATGGCACGAGGAGGAGCAATCTGTAGTAGAAGAGACTTGTGCTCAATCAGCAATAATTCTGGTCCC
ATAGTTTTTAACTATAACCAAGGCGGGAAAGGTGGAGCTATTAGCGCTACCCGATGTGTTATTGACAATAACAAA
GAAAGAATCATCTTTTCAAACAATAGTTCCCTGGGATGGAGCCAATCTTCTTCTGCAAGTAACGGAGGAGCCATT
CAAACGACACAAGGATTTACTTTACGAAATAATAAAGGCTCTATCTACTTCGACAGCAACACTGCTACACACGCC
GGGGGAGCCATTAACTGTGGTTACATTGACATCCGAGATAACGGACCCGTCTATTTTCTAAATAACTCTGCTGCC
TGGGGAGCGGCCTTTAATTTATCGAAACCACGTTCAGCGACAAATTATATCCATACAGGGACAGGCGATATTGTT
TTTAATAATAACGTTGTCTTTACTCTTGACGGTAATTTATTAGGGAAACGGAAACTTTTTCATATTAATAATAAT
GAGATAACACCATATACATTGTCTCTCGGCGCTAAAAAAGATACTCGTATCTATTTTTATGATCTTTTCCAATGG
GAGCGTGTTAAAGAAAATACTAGCAATAACCCACCATCTCCTACCAGTAGAAACACCATTACCGTTAACCCGGAA
ACAGAGTTTTCTGGAGCTGTTGTGTTCTCCTACAATCAAATGTCTAGTGACATACGAACTCTGATGGGTAAAGAA
CACAATTACATTAAAGAAGCCCCAACTACTTTAAAATTCGGAACGCTAGCCATAGAAGATGATGCAGAATTAGAA
ATCTTCAATATCCCGTTTACCCAAAATCCGACTAGCCTTCTTGCTTTAGGAAGCGGCGCTACGCTGACTGTTGGA
AAGCACGGTAAGCTCAATATTACAAATCTTGGTGTTATTTTACCCATTATTCTCAAAGAGGGGAAGAGTCCGCCT
TGTATTCGCGTCAACCCACAAGATATGACCCAAAATACTGGTACCGGCCAAACTCCATCAAGCACAAGTAGTATA
AGCACTCCAATGATTATCTTTAATGGGCGCCTCTCAATTGTAGACGAAAATTATGAATCAGTCTACGACAGTATG
GACCTCTCCAGAGGGAAAGCAGAACAACTAATTCTATCCATAGAAACCACTAATGATGGGCAATTAGACTCCAAT
TGGCAAAGTTCTCTGAATACTTCTCTACTCTCTCCTCCACACTATGGCTATCAAGGTCTATGGACTCCTAATTGG
ATAACAACAACCTATACCATCACGCTTAATAATAATTCTTCAGCTCCAACATCTGCTACCTCCATCGCTGAGCAG
AAAAAAACTAGTGAAACTTTTACTCCTAGTAACACAACTACAGCTAGTATCCCTAATATTAAAGCTTCCGCAGGA
TCAGGCTCTGGATCGGCTTCCAATTCAGGAGAAGTTACGATTACCAAACATACCCTTGTTGTAAACTGGGCACCA
GTCGGCTACATAGTAGATCCTATTCGTAGAGGAGATCTGATAGCCAATAGCTTAGTACATTCAGGAAGAAACATG
ACCATGGGCTTACGATCATTACTCCCGGATAACTCTTGGTTTGCTTTGCAAGGAGCTGCAACAACATTATTTACA
AAACAACAAAAACGTTTGAGTTATCATGGCTACTCTTCTGCATCAAAGGGGTATACCGTCTCTTCTCAAGCATCA
GGAGCTCATGGTCATAAGTTTCTTCTTTCCTTCTCCCAGTCATCTGATAAGATGAAAGAAAAAGAAACAAATAAC
CGCCTTTCTTCTCGTTACTATCTTTCTGCTTTATGTTTCGAACATCCTATGTTTGATCGCATTGCTCTTATCGGA
GCAGCAGCTTGCAATTATGGAACACATAACATGCGGAGTTTCTATGGAACTAAAAAATCTTCTAAAGGGAAATTT
CACTCTACAACCTTAGGAGCTTCTCTTCGCTGTGAACTACGCGATAGTATGCCTTTACGATCAATAATGCTCACC
CCATTTGCTCAGGCTTTATTCTCTCGAACAGAACCAGCTTCTATCCGAGAAAGCGGTGATCTAGCTAGATTATTT
ACATTAGAGCAAGCCCATACTGCCGTTGTCTCTCCAATAGGAATCAAAGGAGCTTATTCTTCTGATACATGGCCA
ACACTCTCTTGGGAAATGGAACTAGCTTACCAACCCACCCTCTACTGGAAACGTCCTCTACTCAACACACTATTA
ATCCAAAATAACGGTTCTTGGGTCACCACAAATACCCCATTAGCTAAACATTCCTTTTATGGGAGAGGTTCTCAC
TCCCTCAAATTTTCTCATCTGAAACTATTTGCTAACTATCAAGCAGAAGTGGCTACTTCCACTGTCTCACACTAC
ATCAATGCAGGAGGAGCTCTGGTCTTTTAA
SEQ ID NO: 83 E. coli RlpB signal sequence (lipidation sequence)
MRYLATLLLSLAVLITAG[C]

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antigen, any method of administration, any prophylactic and/or therapeutic application, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

What is claimed is:

1. An immunogenic composition comprising one or more isolated chlamydia antigens selected from the group consisting of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, a CT476 polypeptide antigen, and combinations thereof.

2.-10. (canceled)

10. The composition of claim 1, wherein the chlamydia antigen has an amino acid sequence selected from SEQ ID NO:1, SEQ ID NO:3, SEQ ID NO:5, SEQ ID NO:7, SEQ ID NO:9, SEQ ID NO:11, SEQ ID NO:23, SEQ ID NO:63, or a portion thereof.

11.-19. (canceled)

20. The composition of claim 1, wherein the composition comprises two or more chlamydia antigens.

21. The composition of claim 20, wherein the two or more isolated chlamydia antigens comprise two or more of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, or a CT476 polypeptide antigen.

22. The composition of claim 20, wherein the two or more isolated chlamydia antigens comprise three or more of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, or a CT476 polypeptide antigen.

23. The composition of claim 20, wherein the two or more isolated chlamydia antigens comprise four or more of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, or a CT476 polypeptide antigen.

24. The composition of claim 20, wherein the two or more isolated chlamydia antigens comprise five or more of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, or a CT476 polypeptide antigen.

25. The composition of claim 20, wherein the two or more isolated chlamydia antigens comprise six or more of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, or a CT476 polypeptide antigen.

26. The composition of claim 20, wherein the two or more isolated chlamydia antigens comprise seven or more of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, or a CT476 polypeptide antigen.

27. (canceled)

28. The composition of claim 20, wherein the two or more isolated chlamydia antigens comprise (a) a first chlamydia antigen selected from a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, and a CT476 polypeptide antigen; and (b) one or more additional chlamydia antigens.

29. The composition of claim 28, wherein the one or more additional chlamydia antigens comprise an antigen selected from the group consisting of a CT856 polypeptide antigen, a CT757 polypeptide antigen, a CT564 polypeptide antigen, a CT703 polypeptide antigen, a P1-ORF7 polypeptide antigen, a CT067 polypeptide antigen, a CT037 polypeptide antigen, a CT252 polypeptide antigen, a CT064 polypeptide antigen, a CT137 polypeptide antigen, a CT204 polypeptide antigen, a CT634 polypeptide antigen, a CT635 polypeptide antigen, a CT366 polypeptide antigen, a CT140 polypeptide antigen, a CT142 polypeptide antigen, a CT242 polypeptide antigen, a CT843 polypeptide antigen, a CT328 polypeptide antigen, a CT188 polypeptide antigen, a CT578 polypeptide antigen, a CT724 polypeptide antigen, a CT722 polypeptide antigen, a CT732 polypeptide antigen, a CT788 polypeptide antigen, and combinations thereof.

30. The composition of claim 28, wherein the one or more additional chlamydia antigens comprise an antigen selected from the group consisting of a p6 polypeptide antigen, a CT310 polypeptide antigen, a CT638 polypeptide antigen, a CT172 polypeptide antigen, a CT443 polypeptide antigen, a CT525 polypeptide antigen, a CT606 polypeptide antigen, a CT648 polypeptide antigen, a CT870 polypeptide antigen, and combinations thereof.

31. The composition of claim 28, wherein the one or more additional chlamydia antigens comprise (a) an antigen selected from the group consisting of a CT856 polypeptide antigen, a CT757 polypeptide antigen, a CT564 polypeptide antigen, a CT703 polypeptide antigen, a P1-ORF7 polypeptide antigen, a CT067 polypeptide antigen, a CT037 polypeptide antigen, a CT252 polypeptide antigen, a CT064 polypeptide antigen, a CT137 polypeptide antigen, a CT204 polypeptide antigen, a CT634 polypeptide antigen, a CT635 polypeptide antigen, a CT366 polypeptide antigen, a CT140 polypeptide antigen, a CT142 polypeptide antigen, a CT242 polypeptide antigen, a CT843 polypeptide antigen, a CT328 polypeptide antigen, a CT188 polypeptide antigen, a CT578 polypeptide antigen, a CT724 polypeptide antigen, a CT722 polypeptide antigen, a CT732 polypeptide antigen, a CT788 polypeptide antigen, and combinations thereof; and (b) an antigen selected from the group consisting of a p6 polypeptide antigen, a CT310 polypeptide antigen, a CT638 polypeptide antigen, a CT172 polypeptide antigen, a CT443 polypeptide antigen, a CT525 polypeptide antigen, a CT606 polypeptide antigen, a CT648 polypeptide antigen, a CT870 polypeptide antigen, and combinations thereof.

32. The composition of claim 21, wherein the composition further comprises one or more additional chlamydia antigens.

33. The composition of claim 32, wherein the one or more additional chlamydia antigens comprise an antigen selected from the group consisting of a CT856 polypeptide antigen, a CT757 polypeptide antigen, a CT564 polypeptide antigen, a CT703 polypeptide antigen, a P1-ORF7 polypeptide antigen, a CT067 polypeptide antigen, a CT037 polypeptide antigen, a CT252 polypeptide antigen, a CT064 polypeptide antigen, a CT137 polypeptide antigen, a CT204 polypeptide antigen, a CT634 polypeptide antigen, a CT635 polypeptide antigen, a CT366 polypeptide antigen, a CT140 polypeptide antigen, a CT142 polypeptide antigen, a CT242 polypeptide antigen, a CT843 polypeptide antigen, a CT328 polypeptide antigen, a CT188 polypeptide antigen, a CT578 polypeptide antigen, a CT724 polypeptide antigen, a CT722 polypeptide antigen, a CT732 polypeptide antigen, a CT788 polypeptide antigen, and combinations thereof.

34. The composition of claim 32, wherein the one or more additional chlamydia antigens comprise an antigen selected from the group consisting of a p6 polypeptide antigen, a CT310 polypeptide antigen, a CT638 polypeptide antigen, a CT172 polypeptide antigen, a CT443 polypeptide antigen, a CT525 polypeptide antigen, a CT606 polypeptide antigen, a CT648 polypeptide antigen, a CT870 polypeptide antigen, and combinations thereof.

35. The composition of claim 32, wherein the one or more additional chlamydia antigens comprise (a) an antigen selected from the group consisting of a CT856 polypeptide antigen, a CT757 polypeptide antigen, a CT564 polypeptide antigen, a CT703 polypeptide antigen, a P1-ORF7 polypeptide antigen, a CT067 polypeptide antigen, a CT037 polypeptide antigen, a CT252 polypeptide antigen, a CT064 polypeptide antigen, a CT137 polypeptide antigen, a CT204 polypeptide antigen, a CT634 polypeptide antigen, a CT635 polypeptide antigen, a CT366 polypeptide antigen, a CT140 polypeptide antigen, a CT142 polypeptide antigen, a CT242 polypeptide antigen, a CT843 polypeptide antigen, a CT328 polypeptide antigen, a CT188 polypeptide antigen, a CT578 polypeptide antigen, a CT724 polypeptide antigen, a CT722 polypeptide antigen, a CT732 polypeptide antigen, a CT788 polypeptide antigen, and combinations thereof; and (b) an antigen selected from the group consisting of a p6 polypeptide antigen, a CT310 polypeptide antigen, a CT638 polypeptide antigen, a CT172 polypeptide antigen, a CT443 polypeptide antigen, a CT525 polypeptide antigen, a CT606 polypeptide antigen, a CT648 polypeptide antigen, a CT870 polypeptide antigen, and combinations thereof.

36.-41. (canceled)

42. A method for eliciting an immune response against chlamydia in a mammal, the method comprising administering to the mammal an immunogenic composition comprising one or more isolated chlamydia antigens selected from the group consisting of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, a CT476 polypeptide antigen, and combinations thereof.

43.-89. (canceled)

89. An isolated nucleic acid comprising a nucleotide sequence encoding a chlamydia antigen selected from the group consisting of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, and a CT476 polypeptide antigen.

90.-94. (canceled)

95. A method for eliciting an immune response against chlamydia in a mammal, the method comprising administering to the mammal a composition comprising one or more nucleic acids encoding one or more chlamydia antigens selected from the group consisting of a CT062 polypeptide antigen, a CT572 polypeptide antigen, a CT043 polypeptide antigen, a CT570 polypeptide antigen, a CT177 polypeptide antigen, a CT725 polypeptide antigen, a CT067 polypeptide antigen, a CT476 polypeptide antigen, and combinations thereof.

96. A kit comprising one or more isolated chlamydia antigens selected from the group consisting of a CT062 polypeptide, a CT572 polypeptide antigen, a CT043 polypeptide antigen, a CT570 polypeptide antigen, a CT177 polypeptide antigen, a CT725 polypeptide antigen, a CT067 polypeptide antigen, a CT476 polypeptide antigen, and combinations thereof.


SEQUENCE LISTING (83 sequences)

SEQ ID NO: 1 - protein, 412 aa, Chlamydia trachomatis
MQQLIDNLKKRGILDNSSAGLESLTVPVSAYLGFDPTAPSLHIGHWIGICFLRRLAAYGITPVALVGGATGMIGDPSGKSVERSLLDQAQVLDNSKKIAAALASYLPGIRIVNNADWLGSLSMVDFLRDVGKHFRLGSMLAKDVVKQRVYSEEGISYTEFSYLLLQSYDFAHLFKEHNVVLQCGGSDQWGNITSGIDYIRRRGLGQAYGLTYPLLTDSKGKKIGKTESGTIWLDPALTPPYELFQYFLRLPDQEISKVMRTLTLLDNEEIFALDERLTSDPQAVKKYIAEVIVKDVHGSEGLAQAQAATESFFASKGKSITEAELVALVESGVGVKVARADLIGKRWLDIVVELGFCSSRGQARRLIQQRGLYINQEPLADEQSILDGTQLCFDRYVLLSQGKRKKQVIDLN

SEQ ID NO: 2 - DNA, 1239 bp, Chlamydia trachomatis
atgcaacagttaatcgataaccttaagaaacggggtattctagataattcttctgcaggattagaaagtttaacagttcctgtttctgcctatttagggttcgatccaactgcgccttctttacacataggacattggattggaatttgttttttgcgtcgattagcagcatatggaatcactcctgttgctcttgttggcggagctaccggaatgatcggagatccttctggtaaaagtgtggagcgttcattactagatcaggcacaggtgcttgataatagtaagaaaatagcggctgctcttgctagctatcttcctggtatccgtattgtgaataatgcggattggctaggatctttaagtatggtggattttttaagagatgttgggaagcattttcgtttaggttctatgttagctaaagacgtagtgaagcagcgagtctattctgaagagggaattagctacactgagttcagttatttattgctgcagtcttatgattttgcacatctctttaaagagcataatgttgtattacagtgtggagggagtgatcagtgggggaatattacttcggggattgattatatccgtcgaagaggactagggcaggcttatggtctaacctatcctttgctcactgatagcaaagggaagaaaatagggaagacggagtctggaactatctggctggatccagcgttaactcctccttatgaactattccaatatttcttacgcttgccagatcaagaaatctccaaagtaatgagaactcttactcttttggataacgaagaaatttttgctcttgatgagcgtttgactagtgatccacaagctgtgaagaaatacattgcggaagtgatcgttaaagatgttcatggttctgagggattagctcaggctcaagccgcaaccgaaagcttttttgctagtaagggaaagagtattacagaagcagaactagtagcgttagtagagtcaggtgttggcgttaaagtagctcgagcagatttaatagggaaacgctggttagatatcgttgtggaactaggcttttgttcctcaagaggacaagctagaagactcattcaacagcgaggtctgtacatcaatcaggagcctttggccgatgaacagagtatattagacgggactcagttgtgtttcgatcgttatgttttgttgtcccaagggaaaagaaaaaaacaagtgatagatcttaattag

SEQ ID NO: 3 - protein, 760 aa, Chlamydia trachomatis
MKNILGYGFLGTFCLGSLTVPSFSITITEKLASLEGKTESLAPFSHISSFNAELKEANDVLKSLYEEALSLRSRGETSQAVWDELRSRLIGAKQRIRSLEDLWSVEVAERGGDPEDYALWNHPETTIYNLVSDYGDEQSIYVIPQNVGAMRITAMSKLVVPKEGFEECLSLLLMRLGIGIRQVSPWIKELYLTNREESGVLGIFGSRQELDSLPMTAHIAFVLSSKNLDARADVQALRKFANSDTMLIDFIGGKVWLFGAVSEITELLKIYEFLQSDNIRQEHRIVSLSKIEPLEMLAILKAAFREDLAKEGEDSSGVGLKVVPLQNHGRSLFLSGALPIVQKAIDLIRELEEGIESPTDKTVFWYHVKHSDPQELAALLSQVHDIFSNGAFGASSSCDTGVVSSKAGSSSNGLAVHIDTSLGSSVKEGSAKYGSFIADSKTGTLIMVIEKEALPKIKMLLKKLDVPKKMVRIEVLLFERKLSNQRKSGLNLLRLGEEVCKQGTQAVSWASGGILEFLFKGGAKGIVPSYDFAYQFLMAQEDVRINASPSVVTMNQTPARIAIVEEMSIVVSSDKDKAQYNRAQYGIMIKILPVINIGEEDGKSFITLETDITFDSTGRNHADRPDVTRRNITNKVRIQDGETVIIGGLRCNQTMDSRDGIPFLGELPGIGKLFGMDSASDSQTEMFMFITPKILDNPSETEEKLECAFLAARPGENDDFLRALVAGQQAAKQAIERKESTVWGEESSGSRGRVEYDGRE

SEQ ID NO: 4 - DNA, 2283 bp, Chlamydia trachomatis
ttattcccgtccatcatactccacccttcctcgagagccggaggattcttctccccatacggtagactcttttctttctatagcctgtttagcagcctgctgtcctgctactaaagctctgaggaaatcatcgttctccccggggcgagcagccaggaaagcacattctaatttttcttctgtctcactaggattatccaaaatcttcggagtgataaacataaacatctctgtttgtgagtccgaagcagaatccataccaaataattttcctattcctggcaactctcctaaaaatggaatcccgtcacgagaatccatagtttgattacaacgaagccccccaataatgaccgtttcgccatcttgaatccgaaccttgttcgtaatatttctgcgtgtaacatcgggacgatccgcatgatttctcccagtcgaatcaaacgtgatgtcggtctctaaagtaataaagctcttcccatcctcttctccgatattaataacgggaagaatcttaatcataatcccgtattgagctcgattgtattgggctttatccttatcagaagaaactacaattgacatttcttccacaatcgcaattctcgccggggtttggttcatagtcacgacggaaggacttgcattaatacggacatcctcttgcgccatgagaaactgataagcaaagtcataactaggaacaatcccttttgctccacctttgaacaggaactccagaatgcccccacttgcccacgaaacggcttgcgttccctgcttacaaacctcttctcctaaacgcaataggttcaatccagatttacgttgattggatagttttctttcaaaaagcagaacctctatacgtaccatttttttgggcacatccagtttcttcaacaacatcttgatcttgggtaaagcttctttctcaataaccataatcaaggttccggtcttggaatctgcaataaaactcccatatttcgcagaaccttcttttacggagctccccagcgacgtatctatatgtaccgctaatccattcgaagaggatcccgctttacttgagactacgccagtatcacaactactagatgccccaaaagcaccatttgagaaaatatcatgtacttgagaaagaagcgctgcaagctcctgaggatctgagtgtttgacatgataccaaaataccgttttgtcggtagggctctctatcccctcttctagttcccgaataagatctattgccttctgaacgatgggaagagctccacttaagaaaagcgagcgtccatggttttgtaaagggaccacttttaatcccactccagaagaatcttctccctctttagctaaatcttctcggaaagctgctttcaaaatagccagcatttctaagggttctatttttgataaagaaacaatgcgatgctcttgtcgaatgttgtctgattgtaagaattcatagattttaaggagctcggtaatctcgctgacagctccaaataaccaaactttcccccctataaaatcaattaacatggtatcgctatttgcgaacttgcgcaaagcttgtacatccgctcgtgcatctaaatttttagaagaaagtacaaaagcaatatgtgccgtcataggcaagctatctagctcttgtctagatccaaagatacctaaaacaccagactcttccctattagttaaatacagctccttaatccaaggactaacctgtctgatcccaatacccagccgcattaaaagcaaagacaaacattcctcaaatccttctttagggaccactagcttagacatggctgtgatacgcatcgccccaacattttgaggaatcacatagatactctgttcatctccgtaatcactgaccagattataaatcgtagtttctggatgattccaaagggcatagtcttcgggatccccccccctttctgcaacctctactgaccataaatcttccaatgaacgtatccgttgtttagcgccgatcaatcggcttcgcaactcgtcccataccgcctgcgaagtctctcctcgagaacggagagacaaagcttcttcgtataaagatttgagaacatcatttgcctctttcaattcagcattaaaagatgaaatatgcgaaaaaggggctagcgattccgtttttccttctagagaagccaatttttctgtaatcgtgatggaaaaactaggaaccgtcaaacttcccaaacaaaaagtccctagaaacccatagcccaaaatatttttcac

SEQ ID NO: 5 - protein, 167 aa, Chlamydia trachomatis
MSRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRA

SEQ ID NO: 6 - DNA, 504 bp, Chlamydia trachomatis
ttatgcacggattcctgctggaggaggttgaattcctccgccacccccttgaggcatttgtggcatggtatcaacagtgggttctcgtccagcgctgatatcagaacaaacagttcgccatttcacaacggtttcaataaaaagctgtgcaaaagctttgagtaggttggtctctgcatacttcatgtctaacacgcagtgcattaagatcaactgttccttagtagcgactcctacccctccaccagccatttggcctccgagcatagagccttctaacaacttctcatatagagctaaccttctttgcggattgtctggcagtccgtcaagaagaggtgcgtaaacataaaggcgatcagagtgttcttcgtaggtcaggtgaagagaaaactctccatcaacaaacaaaatgcacgtattattctgatcgaaggccacgtcggggagtttaagctctttagcaaaattttttagattttcctcagcattctgcctggacat

SEQ ID NO: 7 - protein, 391 aa, Chlamydia trachomatis
MARFLCTYLDQSEKKRRSFVEAFHQREARELLAAQGAHILDIRKVRERNYRVTTTELVIFTKQLVLLLRSGISLYDALTSLRDQYQGRALAGVLTSLMEALRSGGVFSEALARFPHIFDSFYQNSVRSGESIGNLEGALMNIIKVLEEKEKLSKSLAAALSYPVILLVFSCAVVVFFLIGVIPTLKETFEDMEMTRLTKAVFSCSTWFCRYKFLVLLGGIGGAISLRIVWKKRIGKRTLEAIIKKIPILRSLVIKIGFCRFCSVTSAVLQGGGNLIEALTLGCEAVSQDFLREELQEVIQAVVRGGSLSRELSHRTWTPKLVIGMVALGEESGDLAVVFAHVAQIYNEDIQRVLTWVTAWCQPIVLVLLGGFIGLIMLSILLPLTSGIQTF

SEQ ID NO: 8 - DNA, 1176 bp, Chlamydia trachomatis
ttaaaacgtttgaataccgcttgttaacggaagaaggattgataacataatcaatccaataaaaccgcctagcaacacaagaactatgggctgacaccaggcagttacccaagtcaataccctttgaatatcctcgttataaatttgcgcgacatgcgcgaataccaccgcaagatccccggattcttctcctagagcaaccatcccaatcaccagttttggcgtccatgtacgatgagatagctcacgactcaaagatcctccacgaacaactgcttggatcacttcttgtagctcttcgcgcaaaaagtcttgtgatacggcctcgcatcctaatgtcagagcttcgatcaaattcccgcctccttgcaaaacagcagatgtgacggaacaaaatcgacaaaatcctattttaatcaccagactacgcaaaatagggatcttcttgataattgcctctagagtccttttccctatccgttttttccagactatgcgtagggatatcgctccacctattcctcccagcaaaacaagaaacttgtacctacaaaaccatgtactgcacgagaaaacagcttttgtgagccttgtcatctccatatcttcaaaagtttctttcaatgtaggaatgacccctattagaaagaacaccacaacagcacaagaaaataccaataagatcactggataactcaatgctgcagcaagacttttggatagtttttccttctcttccaacactttaataatattcattaaagcgccttctagattcccaatactctctccagaacgcacactattctgataaaaagaatcaaaaatatgcgggaacctcgctagagcttctgaaaagaccccaccggaacgtagagcttccatcaaagaagtgagaaccccagccagcgcacgtccctgatactgatctcgcaatgaagtcaaagcatcgtataaggagatccccgatcgtaataataacactaattgcttagtaaaaataaccagctctgtagttgtgacacggtagtttctctctcgcacctttcgaatgtccagaatgtgagctccttgagcagcaagaagctctcttgcctctcgctgatggaaagcctctacaaaagaacgtcgttttttctcggactgatcaagatatgtacaaagaaacctagccat

SEQ ID NO: 9 - protein, 238 aa, Chlamydia trachomatis
MDTRTPLRKKILIISTALGFVLCVGLMIHTKRSIMPPKTHIPTTAKYFPTIGDPYAPINITVFEEPSCSACEEFSSEVFPLIKKHFVDTGEASLTLVPVCFIRGSMPAAQALLCVYHHDPKRPDPEAYMEYFHRILTYKKTKGSHWATPEVLAKLAEKIPTHSGREINLKGLIQCINSQRFTEQLKKNNIYGSQIMGGQLATPTAVVGDYLIEDPTFDEIERVITQLRHLQAIEEEVR

SEQ ID NO: 10 - DNA, 717 bp, Chlamydia trachomatis
tcaccggacctcctcttctatcgcttgtagatgacgcagttgagtaatcactctctcgatctcatcaaaagtgggatcttcaataagataatctcctacgactgcagtaggtgttgcaagttgcccacccatgatttgagatccatagatattgttctttttaagctgctccgtaaatctttgagaatttatgcactgtattaaacctttgagattaatttctcttccggaatgcgtagggatcttttctgctaattttgcaagcacttcaggagttgcccagtgtgatcctttcgtttttttatatgtgagaattctgtggaaatattccatatatgcttctggatctggacgcttcggatcgtgatggtaaacgcacagtaatgcttgtgcagcaggcattgagccacgaataaaacatacaggaactaaagtcagagaagcttcaccagtgtcaacaaaatgttttttaatcaaaggaaatacttccgaagaaaactcttcacaggcagaacaagatggttcttcaaaaacggtgatattaataggtgcataaggatcccctatcgtagggaaatactttgctgtggttggaatatgcgtctttggtggcataatcgaacgcttagtgtgtatcattaatcctacacacaaaacaaatcctagtgccgtagaaataataaggatcttctttctcaagggagttctcgtatccat

SEQ ID NO: 11 - protein, 184 aa, Chlamydia trachomatis
MKEIYYEIARTESTNTTAKEGLSLWDPYALTVITTREQTAGRGKFGRVWHSTDQDLLASFCFFLSVNNVDSALLFRIGTEAVMRLGESLGIQEAVMKWPNDVLVQGKKLSGVLCETIPVKTGTCVIIGIGVNGNVGADELLGIDQPATSLQELIGRPVDMEEQLKRLTKEIKHLIQTLPLWGRE

SEQ ID NO: 12 - DNA, 555 bp, Chlamydia trachomatis
atgaaagaaatctattatgaaatagcacgtacggaatcaacgaatacgacagcaaaagaggggctttctttgtgggatccctatgctctcacagtgatcacgaccagagaacaaacggcgggaagagggaaatttggaagggtctggcactccacagatcaagatcttttggcttcgttttgtttctttttaagtgtgaataatgtggacagtgctttgttatttcgtatagggacagaagccgtgatgcgtctcggggaatcgttaggcattcaagaagctgtcatgaaatggcctaacgacgtgttagttcaggggaaaaaactttcaggagtgttgtgtgagaccatccctgttaagactggaacgtgtgtcattattggtatcggtgtgaatggtaatgtgggtgctgatgaattgctaggtattgatcagcctgcaacgtctctccaggaattgatagggaggcctgtagatatggaagaacagcttaagcggctcacgaaagaaatcaagcatcttatccagacgctaccgttatgggggcgagaataa

SEQ ID NO: 13 - protein, 567 aa, Chlamydia trachomatis
MVKVSLSFKHLVPKLVTCLKEGYSFNTLKKDFTAGITAGILAFPLAIAIAIGIGVSPLQGLLASIIGGFLASALGGSRVLISGPTSSFISILYCIGVKYGEDGLFTITLMAGIFLIIFGLAGLGTFIKYMPYPVVTGLTTGIAVIIFSSQIRDFLGLQMGDGVPLDFIGKWAAYWDYLWTWDSKTFAVGLFTLLLMIYFRNYKPRYPGVMISIIIASTLVWILKIDIPTIGSRYGTLPSSLPGPVFPHISITKMLQLMPDALTISVLSGIETLLAAVVADGMTGWRHQSNCQLIGQGIANIGTSLFAGMPVTGSLSRTTASIKCGASTPIAGIIHAICLSFILLLLAPLTIKIPLTCLAAVLILIAWNMSEIHHFIHLFTAPKKDVVVLLTVFILTVMTTITSAVQVGMMLAAFLFMKQMSDLSDVISTAKYFDESEQPQNDLLFSKNEVPPFTEIYEINGPFFFGIADRLKNLLNEIEKPPKIFILCMTRVPTIDASAMHALEEFFLECDRQGTLLLLAGVKKTPLSDLRRYHVDELIGVDHIFPNIKGALLFAKALIKLESKSSQ

SEQ ID NO: 14 - DNA, 1704 bp, Chlamydia trachomatis
ctattgagaagacttactctctaacttaataagggcttttgcaaacaataacgcacctttaatgtttgggaagatatggtctactccgatcaattcatctacatggtaccttctcaaatcactgagaggagtttttttcacgccagctaagagaagcaatgttccttgtcggtcgcattccaagaagaactcttctagagcgtgcatggcagatgcatctattgtaggcactcgagtcatgcaaaggataaatattttaggcggcttttctatttcatttaataagtttttcaaacgatctgcgatgccaaagaaaaacggtccgttgatttcataaatttccgtaaaaggtggtacttcatttttgctaaatagcaagtcattttgaggttgttcggattcatcaaaatattttgctgtggagataacatcagatagatcgctcatttgtttcatgaatagaaaggctgcaagcatcattcctacttgtactgcagaagtaatcgtagtcattactgtaagaatgaacacggttagcaggacaacaacgtcttttttaggagctgtgaatagatgaatgaaatggtgaatttcactcatattccaagcaattaaaattaaaacagctgctagacatgttagagggattttaatagttaagggagctaggagtagtaggataaaggaaagacagatggcatggattattcctgctataggagtactagcgccgcacttgatgctagccgttgttcttgaaagcgagcctgtaacaggcatgccagcaaataaagaggttccaatgttagcaattccttggccaattaattggcagttggattgatgtctccacccagtcattccatctgcaacgacagctgctaataaggtttctattccagaaagaacggaaatagttaaagcatctggcataagttgaagcattttagtaatgcttatgtgtgggaaaactggaccaggtaaagagcttggtaaggtaccataacggctaccgatggtagggatgtctattttaagaatccatactagagtcgatgcaatgataatagaaatcattacgccgggataacgaggtttgtaattgcgaaagtagatcattagaagcagggtaaataaacccacagcaaaggtcttgctatcccaggtccataggtaatcccaataggctgcccatttgccgatgaagtctaaaggaactccatctcccatttgaagcccaagaaaatctcggatttgggaagaaaaaatgatgaccgcaattcccgtagttagtccggtcaccacaggatacggcatatatttaataaaagtgcctagtccggcaagaccaaagataatgaggaagatcccagccatcaatgtgatagtaaacagtccgtcttcgccatatttgacaccgatacagtaaaggatggagataaaggaactggtagggccagagattaatacacgactgcctcctaaggcagaggctaaaaagcctccaataattgaggccaatagtccttgtaaaggagacactccaatcccgatcgcaatagcaatagctaaagggaaggctagaatccctgcagtgatccctgcggtaaagtcttttttgagcgtattaaaagaatacccttcttttaagcaggtaactaatttagggacaagatgtttgaaggatagggaaactttcaccaa

SEQ ID NO: 15 - protein, 336 aa, Chlamydia trachomatis
MLPLTYVVKAFSIGLFFSLFLMKPLISWLKKQGFQDHIHKDHCEKLEELHKDKAYIPTAGGIVFVFASVLAVLLLFPIQLWSTWFCIGTILLWGALGWCDDQIKNRRRVGHGLSAKHKFLIQNCLAAGVVLPIMFAYKESFLSFHLPFLGIVSLPHHWWSYLLSFAIATLAIVGTSNSVNLTDGLDGLAAGAMVIACLGMLVVACTNGAPWAFICCVLLATLAGSCLGFLRYNKSPARVFMGDTGSLFLGAMLGMCAVLLRAEFLLLFMGGIFVLESLSVIVQVGSYKLRKKRVFLCAPLHHHYEYKGLSEKAVVRNFLIVELICVVVGIIAVFVD

SEQ ID NO: 16 - DNA, 1011 bp, Chlamydia trachomatis
atgctgcccctaacgtatgttgtgaaagccttttctattggcttgttttttagcctttttttgatgaaacctttgatttcttggttaaaaaaacaaggttttcaagatcatattcacaaagatcactgcgaaaaattagaagagttacataaagacaaagcatatatccctacagctggagggatagtttttgtttttgcatctgtgttggcggttcttttattgttccccatacagctttggtctacatggttttgtattggaactattctattatggggagcattaggatggtgcgatgatcagattaaaaatcggcgtagagtagggcatgggttgtctgctaaacataagtttcttatacagaattgtttggctgcaggggtggttcttcctattatgttcgcatataaagaaagttttcttagttttcatcttccttttctaggaatcgtttctttgccacatcattggtggagctatctactcagttttgctattgcaacattggctattgttggaacgagcaattcagtcaatctcactgatggattggatggacttgcggcaggagctatggtgatagcctgcttagggatgcttgtcgttgcttgtactaatggagctccttgggccttcatttgttgtgttcttctagctaccttagctggaagttgtcttggatttttacgttacaacaagtctcctgcccgtgtctttatgggagatacaggatctttgtttttaggagccatgctcggtatgtgtgctgtattattacgagcagagtttcttctcttgtttatgggagggatttttgttctggaatcactatctgtgattgtacaagtcggaagttataaattaagaaagaaacgagtctttctttgtgcccctttacaccatcattatgagtataaggggttatcagaaaaggctgtagtgaggaatttcttaattgtcgagcttatttgtgtagtagttgggatcattgcagtatttgtggattag

SEQ ID NO: 17 - protein, 289 aa, Chlamydia trachomatis
MATLPEVLSGLGSSYIDYIFQKPADYVWTVFLLLAARILSMLSIIPFLGAKLFPSPIKIGIALSWMGLLLPQVIQDSTIVHYQDLDIFYILLIKEILIGVLIGFLFSFPFYAAQSAGSFITNQQGIQGLEGATSLVSIEQTSPHGIFYHYFVTIVFWLAGGHRIILSVLLQSLEIIPLHAVFPESMMSLRAPMWIAILKMCQLCLIMTIQLSAPAAVAMLMSDLFLGIINRMAPQVQVIYLLSALKAFMGLLFLTLAWWFIVKQIDYFTLAWFKEIPTMLFGAHPPKVL

SEQ ID NO: 18 - DNA, 870 bp, Chlamydia trachomatis
atggctacgcttcccgaggttctttcagggctcggctcttcctatatcgattatatattccaaaagccagccgattacgtttggactgtctttcttttgctagcggcacgcatattatctatgctgtcgatcatcccgttcttaggagctaaactattcccgtcaccaattaaaattgggatagcgctctcttggatgggattgctgctacctcaggtgatacaagactctacgatcgtccactaccaagacctagatattttctatatccttcttattaaggagattttgattggcgtactcatcggctttctgttctcttttcccttctatgctgcccagtctgcaggatcctttattaccaaccagcaagggatacaaggattagaaggtgctacctctctcgtatctatagaacaaacttctcctcacgggatcttttatcattattttgtgactatcgttttctggctcgcaggaggacatcgcattatcctttctgttcttttacaatcgcttgagatcatccctcttcatgctgttttccctgagagcatgatgtcgctacgagctcctatgtggatcgcgatattaaaaatgtgccaattgtgcttgattatgaccatacagttgagcgctccagcagcggtggctatgcttatgtcagatttattcctagggatcatcaaccgaatggctcctcaggtacaagtcatctacctactttctgcactgaaagcctttatgggattgttattcctaacactggcttggtggttcattgtgaaacaaattgattatttcactctggcatggttcaaagaaatccctactatgctcttcggagctcatcctcctaaagttttgtga

SEQ ID NO: 19 - protein, 490 aa, Chlamydia trachomatis
MRIAILGRPNVGKSSLFNRLCKRSLAIVNSQEGTTRDRLYGEIRAWDSIIHVIDTGGVDQESTDRFQKQIHQQALAAAEEASVLLLVVDIRCGITKQDEELAKRLLPLKKPLILVMNKADSQQDLQRIHEFYGLGISDMIATSASHDKHIDLLLERIRQIAQIPVPSVEEQDAVQEDELPSEEAAISLHAFADETLFENESLSQEEASFLEELVAQTATPAPVDRPLKVALIGHPNVGKSSIINALLKEERCITDNSPGTTRDNIDVAYTHNNKEYVFIDTAGLRKTKSIKNSVEWMSSSRTEKAISRTDICLLVIDATQQLSYQDKRILSMIARYKKPHVILVNKWDLMFGVRMEHYVQDLRKMDPYIGQARILCISAKQRRNLLQIFSAIDDIYTIATTKLSTSLVNKVLASAMQRHHPQVINGKRLRIYYAIHKTTTPFTFLLFINSNSLLTKPYELYLKNTLKAAFNLYRVPFDLEYKAKPARKSN

SEQ ID NO: 20 - DNA, 1473 bp, Chlamydia trachomatis
ttaatttgattttcttgcaggttttgctttgtattctaaatcaaatggaactctatataaattaaaagctgcttttaaagtgttttttaaatacaactcgtaaggtttcgtcagcagactattggaattgataaacagcaagaaagtaaatggtgtcgtcgtcttatgaatcgcatagtagatgcgtaaacgtttgccattaatgacctgcggatggtgtctttgcatagcagaagctaataccttgttaactaaggaagtcgagagttttgtcgttgcaatagtatagatatcatcaatagcagaaaagatttgtaacagattgcggcgttgcttggctgaaatacaaagtatgcgcgcttgacctatatagggatccatttttcgcaagtcttgaacataatgttccatgcgaacaccaaacattaagtcccatttatttacgagaatcacatgaggttttttatatctcgcaatcatagatagaatccgcttatcttgataggagagctgctgggtcgcatcgatcactaataggcaaatgtctgttctggaaatggctttttctgttcgagaagaagacatccattccacagagtttttaatgctcttagtttttcttaatccggcagtatctataaagacgtattctttattgttatgcgtataggcaacatcgatgttgtctcgtgtagtccctggagaattatccgttatacagcgctcctccttaagaagagcattgataatggaggatttccctacattgggatgcccaatcaacgctacctttaacgggcggtctacaggggctggcgtcgccgtctgcgcaacgagctcttcaaggaaagaagcttcttcttgcgatagggattcattttcaaaaagagtttcatcagcaaaggcatgcaaagatatagcagcctcttcagaggggagctcgtcttcttgtacagcatcttgttcttctacagaaggtacagggatctgcgcgatctgacggatgcgttccaagagtaaatcaatatgcttatcatggctagccgatgtggcaatcatatcagagattcccaatccataaaattcatgaatgcgctgtaaatcctgctgggaatccgctttattcataacaagaatcaaaggcttcttcaacggcaggagacgcttagccagctcttcatcttgtttggtgataccacatcggatatctactacaagcagcagaacagaggcttcctctgctgctgctaaagcctgttgatgaatttgcttttggaatcggtcggtagactcttggtctacgcccccagtatcgataacatggataatagaatcccaggctcgaatttctccatacaaacgatctcgcgtagttccttcttgagagttcacaatcgctaaagagcgtttacataagcggttgaagagagaagacttccctacattgggtcttcctaaaatagcaatacgcat

SEQ ID NO: 21 - protein, 305 aa, Artificial Sequence
  Synthetic P1 - ORF7 polypeptide
MGSMAFHKSRLFLTFGDASEIWLSTLSYLTRKNYASGINFLVSLEILDLSETLIKAISLDHSESLFKIKSLDVFNGKVVSEASKQARAACYISFTKFLYRLTKGYIKPAIPLKDFGNTTFFKIRDKIKTESISKQEWTVFFEALRIVNYRDYLIGKLIVQGIRKLDEILSLRTDDLFFASNQISFRIKKRQNKETKILITFPISLMEELQKYTCGRNGRVFVSKIGIPVTTSQVAHNFRLAEFHSAMKIKITPRVLRASALIHLKQIGLKDEEIMRISCLSSRQSVCSYCSGEEVIPLVQTPTIL

SEQ ID NO: 22 - DNA, 481 bp, Artificial Sequence
  Synthetic P1 - ORF7 DNA
atgggctcgatggctttccataaaagtagattgtttttaacttttggggacgcgtcggaaatttggttatctactttatcttatctaactagaaaaaattatgcgtctgggattaactttcttgtttctttagagattctggatttatcggaaaccttgataaaggctatttctcttgaccacagcgaatctttgtttaaaatcaagtctctagatgtttttaatggaaaagttgtttcagaggcatctaaacaggctagagcggcatgctacatatctttcacaaagtttttgtatagattgaccaagggatatattaaacccgctattccattgaaagattttggaaacactacattttttaaaatccgagacaaaatcaaaacagaatcgatttctaagcaggaatggacagttttttttgaagcgctccggatagtgaattatagagactatttaatcggtaaattgattgtacaag

SEQ ID NO: 23 - protein, 326 aa, Chlamydia trachomatis
MSFFHTRKYKLILRGLLCLAGCFLMNSCSSSRGNQPADESIYVLSMNRMICDCVSRITGDRVKNIVLIDGAIDPHSYEMVKGDEDRMAMSQLIFCNGLGLEHSASLRKHLEGNPKVVDLGQRLLNKNCFDLLSEEGFPDPHIWTDMRVWGAAVKEMAAALIQQFPQYEEDFQKNADQILSEMEELDRWAARSLSTIPEKNRYLVTGHNAFSYFTRRYLSSDAERVSGEWRSRCISPEGLSPEAQISIRDIMRVVEYISANDVEVVFLEDTLNQDALRKIVSCSKSGQKIRLAKSPLYSDNVCDNYFSTFQHNVRTITEELGGTVLE

SEQ ID NO: 24 - DNA, 981 bp, Chlamydia trachomatis
atgtctttttttcatactagaaaatataagcttatcctcagaggactcttgtgtttagcaggctgtttcttaatgaacagctgttcctctagtcgaggaaatcaacccgctgatgaaagcatctatgtcttgtctatgaatcgcatgatttgtgattgcgtgtctcgcataactggggatcgagtcaagaatattgttctgattgatggagcgattgatcctcattcatatgagatggtgaagggggatgaagaccgaatggctatgagccagctgattttttgcaatggtttaggtttagagcattcagctagtttacgtaaacatttagagggtaacccaaaagtcgttgatttaggtcaacgtttgcttaacaaaaactgttttgatcttctgagtgaagaaggattccctgacccacatatttggacggatatgagagtatggggtgctgctgtaaaagagatggctgcggcattaattcaacaatttcctcaatatgaagaagattttcaaaagaatgcggatcagatcttatcagagatggaggaacttgatcgttgggcagcgcgttctctctctacgattcctgaaaaaaatcgctatttagtcacaggccacaatgcgttcagttactttactcgtcggtatctatcctctgatgcggagagagtgtctggggagtggagatcgcgttgcatttctccagaagggttgtctcctgaggctcagattagtatccgagatattatgcgtgtagtggagtatatctctgcaaacgatgtagaagttgtctttttagaggataccttaaatcaagatgctttgagaaagattgtttcttgctctaagagcggacaaaagattcgtctcgctaagtctcctttatatagcgataatgtctgtgataactattttagcacgttccagcacaatgttcgcacaattacagaagaattgggagggactgttcttgaatag

SEQ ID NO: 25 - protein, 118 aa, Chlamydia trachomatis
MESFFVLKIPFFLLNGVQDSPCLSLVLFYSFFPFTLNWFATLGGRPTAPRNSVLIQLKLKKILSTTLVIQESPNTKKAPREYTVRGDFSKLLNFGIIEASEIRKVPMKSALHCTLRED

SEQ ID NO: 26 - DNA, 357 bp, Chlamydia trachomatis
ttaatcctctctaagagtgcaatgcaacgcacttttcatagggacttttcgtatttctgaggcctcaatgatgccaaaattgaggagtttagaaaagtcgcctcggacagtatactcccttggagcttttttagtatttgggctttcctgtattacgagagtggtcgatagaattttttttaattttagctgaattagaacgctatttcgcggtgcagttggtctaccaccaagagttgcaaaccaattgagggtgaacgggaaaaatgaataaaaaaggacgagagagagacagggactatcttgaactccatttagcagaaaaaaaggtattttcaaaacaaaaaaagactccat

SEQ ID NO: 27 - protein, 272 aa, Chlamydia trachomatis
MIHWDQSRTLLSFPRVGLHLSWYGILFSLGIFLSSFSGIKLATALCKDREEKKELRTSLENFALGALLAIIIGARLAYVLFYGGSFYFENPSEIIKIWKGGLSSHGAVISVVIWAAVFSRLHIRKLPMLSVTYICDLCGAVFGCAALLIRVGNFMNQEILGTPTSMPWGVIFPNGGGQIPRHPVQLYEGLGYLVLSCILYRLCYRGVIRLGSGYSAAGALIGVAVIRFCAEFFKTHQGAWLGEENILTIGQWLSIPMIFLGVGIIWIASKKK

SEQ ID NO: 28 - DNA, 819 bp, Chlamydia trachomatis
tcatttttttttactagcaatccaaatgattccaactcctagaaaaatcatcggaatagacaaccattgcccaattgttaatatgttttcttcgccaagccatgctccttggtgtgttttgaaaaattcagcgcaaaaacgaattactgctaccccaattaaagcgcctgctgcactatagccagaacccaaacgaataacaccacgatagcaaagcctgtacagaatacaagaaagcactaaataaccaaggccttcgtaaagctgaacaggatgtctagggatttggcctccaccattcggaaaaatcactccccaaggcatggatgtaggggttcctagaatttcctgattcataaagttccccacgcgaatcagcaaagctgcacaaccaaacactgctccacaaagatcgcaaatgtaggttactgaaagcataggcaacttacgaatatgaagtcgcgaaaatacagctgcccaaatcaccacagagatcacagctccatgactagaaagccctcctttccatatttttataatctcagaaggattttcaaaataaaaactccctccatagaaaagaacgtaagcaagcctagctccaatgatgatagctaaaagagctcctaaagcaaaattttccagacttgttcggagttcttttttctcctccctgtctttacacaatgctgttgccagcttgatgcccgaaaaagatgataaaaaaattcctagagaaaataagattccgtaccacgataaatgaagcccaactcgcgggaaagataagagagttctagactggtcccaatgtatcac

SEQ ID NO: 29 - protein, 602 aa, Chlamydia trachomatis
MKPYKIENIRNFSIIAHIDHGKSTIADRLLESTSTIEQREMREQLLDSMDLERERGITIKAHPVTMTYEYEGETYELNLIDTPGHVDFSYEVSRSLAACEGALLIVDAAQGVQAQSLANVYLALERDLEIIPVLNKIDLPAAQPEAIKKQIEEFIGLDTSNTIACSAKTGQGIPEILESIIRLVPPPKPPQETELKALIFDSHYDPYVGIMVYVRVISGEIKKGDRITFMATKGSSFEVLGIGAFLPEATLMEGSLRAGQVGYFIANLKKVKDVKIGDTVTTVKHPAKEPLEGFKEIKPVVFAGIYPIDSSDFDTLKDALGRLQLNDSALTIEQENSHSLGFGFRCGFLGLLHLEIIFERISREFDLDIIATAPSVIYKVVLKNGKTLFIDNPTAYPDPALIEHMEEPWVHVNIITPQEYLSNIMSLCMDKRGICLKTDMLDQHRLVLSYELPLNEIVSDFNDKLKSVTKGYGSFDYRLGDYKKGAIIKLEILINDEAVDAFSCLVHRDKAESKGRSICEKLVDVIPPQLFKIPIQAAINKKIIARETIRALAKNVTAKCYGGDITRKRKLWDKQKKGKKRMKEFGKVSIPNTAFVEVLKME

SEQ ID NO: 30 - DNA, 1809 bp, Chlamydia trachomatis
ctactccattttaaggacttcaacaaacgccgtgttcggaatggatacttttccgaattctttcattcgtttcttcccttttttctgtttgtcccacaacttgcgttttcttgtgatatctccaccatagcacttagcagttacatttttcgctaaagctcgaatcgtctctctggcaataatctttttattgatggccgcctgaatagggattttaaagagctgaggagggataacatctacgagtttctcgcagatgcttctgccttttgattctgctttgtctctgtgtacaaggcaggaaaaggcatcaacagcctcatcattaattagaatttccagcttaatgatagcaccctttttataatctcctaaccggtaatcaaaggagccgtatcctttcgtcacagatttgagtttatcattgaaatcagaaacaatctcattgagaggcagctcatatgaaagcaccagtctgtgttggtcaagcatatctgtttttagacagatcccacgcttatccatacaaaggctcataatattgctgagatactcttgaggcgtaatgatattaacatggacccaaggctcctccatgtgttcaataagagctgggtcaggatatgctgttgggttatcaataaaaagggttttaccattttttaagacgactttgtagataacgctaggagctgtagcaataatatcgagatcaaattctctagagattctctcaaagatgatttctaagtgcagcagtcctaaaaatccacagcggaacccaaatccgagagaatgactgttctcttgttcaatcgtaagagctgagtcgtttagctgcaaccggcctagagcatctttcagggtatcaaagtcagaagaatctataggatagataccagcaaacactacaggtttgatttctttaaagccttctaaaggctctttagcaggatgtttaacagtagtgactgtatcgccaatttttacatcctttactttttttaggttggcaatgaagtatcccacttgtccggctcgtaaggatccttccatgagagtagcttccggtaagaaagctcctattcctaagacctcaaaagaggagcctttggttgccatgaaggtaatgcgatctccctttttgatttctccactgatcacgcgtacataaaccatgattcctacataaggatcgtagtgagaatcaaagatcaaagctttaagttctgtttcctgtggaggttttggtgggggaacgagtcgtataatagactctaaaatttcagggataccctgacctgttttcgctgagcaagcaatggtgtttgaagtatctaatccgatgaactcttcgatttgtttttttatagcttctggttgagcagcaggtaagtctattttatttaaaacaggaatgatttctaaatctcgttctagagccagatatacattagctaagctttgagcttgaacaccttgggcagcatctactataagcagcgctccttcacaagctgctagtgatcgggatacttcataagagaaatctacgtgtccaggagtatctattagattgagttcgtaagtctccccttcgtattcataggtcatagtgaccggatgcgctttgatggtaatcccgcgttctctttctagatccatagaatctaaaagttgttcgcgcatctctctttgttcgatagtactagtactttctaacaaacgatctgcgatcgtagatttcccgtggtcgatatgagcaatgatagaaaaattacgaatgttctcaattttatacggtttcaa

SEQ ID NO: 31 - protein, 281 aa, Chlamydia trachomatis
MFSQQIEESIKAGQVFAFPTDTVYGLGVSFHILDADQRLFALKHRSSQKALSVYVSSLEELEAVAQQSLGASSRKIIQKFLPGPLTLITKHNNPRFPQKTLGFRIVNHPIVQQIIQKVGPFLATSANLSGFPSAVSADEVKQDFPEEDIVMISGECSIGLESTVIDPEERIVYRESAISIAEIETVLGAPCANLSKELGFREKIGIHVVKTPADLCSFLLSRPHFKGVICHQPHPHTFYSVLRQALRSPTQEIIFVYDLCNTEYPILSRFLGVSYDSGYAL

SEQ ID NO: 32 - DNA, 846 bp, Chlamydia trachomatis
gtgttttcgcaacagattgaggagagcattaaggcggggcaagtttttgccttccctacagatacagtatatggtttgggagtgtcttttcatatccttgatgctgatcagcgattatttgctcttaagcacagatcttcccaaaaagctctgtccgtctatgtctcatctttagaagaattagaggctgttgcccaacagtctttaggagcatcttcgagaaagataattcaaaagtttcttcctgggcctcttaccttgattacaaaacataataatccgagatttcctcagaaaacattgggattcaggattgttaatcatcctatagtgcagcagatcattcaaaaagtagggccgtttcttgctacttcagcgaatctatccggctttccttctgcagtttctgctgatgaggtaaaacaagatttcccggaagaagatatcgtaatgatttcaggagaatgttctatagggttggagtctacagtaatcgatcctgaggagcgaattgtttatcgtgagagtgctatttctattgcagaaatagaaactgtattaggggctccatgtgctaatctgtctaaggaactagggtttagagaaaaaataggtatccatgttgtaaaaacccccgcagatttatgtagttttcttttgtctagacctcattttaagggtgttatttgccatcagcctcatcctcatactttttattctgttctaaggcaggctttacgctctcctacacaagaaatcattttcgtttacgatttgtgcaatacagaatatccaattctttcacgttttctaggagtgagttatgatagtggatatgcattgtga

SEQ ID NO: 33 - protein, 446 aa, Chlamydia trachomatis
MNKHKRFLSLVLLTFILLGIWFCPHSDLIDSKAWHLFAIFTTTIIGIIVQPAPMGAIVIMGISLLLVTKTLTLDQALSGFHSPITWLVFLSFSIAKGVIKTGLGERVAYFFVKILGKSPLGLSYGLVLTDFLLAPAIPSLTARAGGILFPVVMGLSESFGSSVEKGTEKLLGSFLIKVAYQSSVITSAMFLTAMAGNPIISALASHSGVTLTWAIWAKTAILPGIISLACMPFVLFKLFPPQITSCEEAVATAKTRLKEMGPLNQGERIILLIFSLLISLWTFGDSIGISATTTTFIGLSLLILTNILDWQKDVLSNTTAWETFFWFGALIMMASFLSAFGFIHFVGDSVIGSVQGLSWKIGFPILFTVSISLGANPMFAALALAFASNLFGGLTHYGSGPAPLYFGSHFVSVQEWWRSGFILSIVNLTIWLGLGSWWWYCLGLIR

SEQ ID NO: 34 - DNA, 1416 bp, Chlamydia trachomatis
atgaataaacacaaacgcttcttatcgctcgtactcttaacatttatccttctcggaatttggttctgcccgcattctgatctcatcgactccaaagcgtggcacttatttgcgatatttactacgactattatcggaatcattgtacaacccgctcctatgggagccattgttatcatgggcatttctcttctgcttgtgaccaaaacattaactctagatcaagctttgtccggatttcatagccctattacttggcttgtatttctttcgttttccatagcaaaaggcgtgattaaaacaggtcttggagagcgagttgcttacttctttgtaaaaatattgggtaaaagtcctttaggattgagctatggcttagttcttacagactttttattagcaccggcaatccctagtttgacagctcgcgctggaggcattcttttccctgttgttatgggattatcagagtctttcggtagttctgtagaaaaaggcacggaaaaacttctcggatcttttttaatcaaagtagcttatcaaagctctgtaattacaagtgctatgtttttaactgctatggctggaaaccctattatttctgccttagcaagtcattctggagtaacgttaacatgggcaatttgggctaaaaccgcaatccttccagggattattagcttagcctgtatgccttttgtactctttaaactattcccaccacaaataactagctgtgaagaagctgtagcaactgccaaaactcgcttaaaagaaatgggacctttaaatcaaggcgaacgcattattcttttaatcttttctcttttaatatctttatggactttcggagattccatcggcatctcagcaacaaccacaacatttataggactatccctactcattcttacgaatattcttgattggcaaaaagatgttctttctaacactactgcatgggaaacctttttctggttcggagctttaattatgatggcttccttcctaagcgcttttgggtttattcattttgtaggagattctgttattgggagcgttcaaggtctatcttggaaaatagggttccctatactctttcttatttatttctactctcactatctatttgcgagtaatacagcacatattgcagccatgtaccctatctttcttacagtatccatctccttaggcgcgaatcctatgtttgctgccttagccttagcttttgctagtaatttattcggaggactcacacactacggatctggtccagctccgttatactttggatcccatttcgtctccgtgcaagaatggtggcgctctggctttattcttagcatagtcaatctaaccatttggttgggattaggaagttggtggtggtactgtttaggattaattcgctaa

SEQ ID NO: 35 - protein, 465 aa, Chlamydia trachomatis
MKIVVSRGLDLSLKGAPKESGFCGKVDPTYVSVDLRPFAPLPLGVKVTPEDQVTAGSPLAEYKLFSGVFITSPVDGEVVEIRRGNKRALLEIVIKKKPGISQTKFSYDLQSLTQKDLLEVFKKEGLFALFKQRPFDIPALPTQSPRDVFINLADNRPFTPSVEKHLSLFSSKEDGYYIFVVGVQAIAKLFGLKPHIISTDRLTLPTQDLVSIAHLHTIDGPFPSGSPSTHIHHIARIRNERDVVFTISFQEVLSIGHLFLKGFVLGQQIVALAGSALPPSQRKYLITAKGASFSDLLPKDIFSSDEITLISGDPLTGRLCKKEENPCLGMRDHTITLLPNPKTRESFSFLRLGWNKLTVTRTYLSGFFKRKRVFMDMDTNMHGEKRPIIDAEIYERVSAIPVPVALIIKALETQNFEEACRLGLLEVAPEDFALPTFIDPSKTEMFSIVKESLLRYAKENVVTSS

SEQ ID NO: 36 - DNA, 1398 bp, Chlamydia trachomatis
ttacgaggaggttaccacattctcttttgcgtagcgtaaaagagattctttgacgatagagaacatctcggtcttagaaggatctatgaatgtggggagagcaaaatcttctggagcaacttctaagagccctaggcgacacgcttcttcaaagttttgtgtttccaaagctttaataataagagctacaggaaccgggattgctgaaacacgctcatagatttcagcatcaataatgggccgtttttctccatgcatgttagtatccatatccatgaagacccgttttctcttgaaaaaaccagatagataggttcgtgtgactgtaagtttattccaacctaagcgcaagaaactgaaagattcacgagttttaggattaggaagaagtgttatggtatggtctctcatacctaaacaaggattttcttcttttttacataatcttcctgtaagaggatctccagaaataagggtaatctcatcggaagagaaaatgtctttaggaagaagatcagagaaactagcgcctttcgcagtaatgagatattttctttgagaaggaggaagagctgatcctgctaaggcaacgatttgttgtcctaaaacaaagccttttaaaaatagatgccctatagataacacctcttggaagctaatagtaaacacaacatctctttcgtttcgaatacgagcgatgtgatgaatgtgcgttgaaggagatcctgatgggaaggggccatctattgtgtgtaagtgggctatggatacgagatcctgggttgggagagttagtctgtctgtagaaatgatatgaggcttcagtccaaatagttttgctattgcctgaactcccacaacaaaaatgtaataaccatcttcttttgaagaaaaaagactgagatgtttttccacagaaggggtgaaagggcgattatccgctaagttaataaaaacatctcgaggagattgtgttggaagagctgggatatcaaaaggtctttgtttgaaaagagcgaaaagaccttcctttttaaaaacttctaaaagatctttttgagtcaaagattgaagatcataagaaaacttagtttgagaaataccaggcttcttcttgatgacgatctctaaaagagcacgtttatttcctctacggatctctacaacctctccatcaacaggagaggtaataaacactcctgaaaaaagcttgtactcagccaggggagaaccagcagtaacttggtcttctggagtaacctttacccctaaaggaaggggagcgaaaggcctcaaatccacggaaacataggtggggtccaccttaccgcaaaaacccgattccttcggagctccctttaaagacagatctaatccgcgagaaacaactattttcat

SEQ ID NO: 37 - protein, 144 aa, Chlamydia trachomatis
MKNNSAQKIIDSIKQILSIYKIDFEPSFGATLTDDNDLDYQMLIEKTQEKIQELDKRSQEILQQTGMTREQMEVFANNPDNFSPEEWRALENIRSSCNEYKKETEELIKEVTNDIGHSSHKSPTPKKTKSSSQKKSKKKNWIPL

SEQ ID NO: 38 - DNA, 435 bp, Chlamydia trachomatis
ttataagggaatccaattttttttcttactttttttctgagaggaggattttgtcttttttggcgttggagatttgtgggatgagtgaccaatatcattggttacttctttgataagctcttcagtttcttttttgtattcattgcaagaggaacgaatgttttctagagctcgccactcttcaggagaaaagttatctggattattagcaaagacttccatttgttcgcgagtcattcccgtctgttggagaatttcctgcgatcttttgtctaattcttggattttttcctgtgttttttcgatcagcatttggtagtccagatcgttgtcgtcagtaagagttgctccaaaggagggttcgaagtctattttataaatagagagaatttgttttatagaatctataattttttgagcggaattatttttcat

SEQ ID NO: 39 - protein, 184 aa, Chlamydia trachomatis
MPTFDTTKQIFLCGLPSVGKTSFGQHLSQFLSLPFFDTDHLLSDRFHGDSPKTIYQRYGEEGFCREEFLALTSVPVIPSIVALGGCTPIIEPSYAHILGRNSALLVLLELPIATLCQRLQHRSIPERLAHAPSLEDTLSQRLDKLRSLTSNAFSLRAETSSEAVMRDCQSFCLRFLSTKESSYA

SEQ ID NO: 40 - DNA, 1323 bp, Chlamydia trachomatis
atggtctcttcgaaccaagaccttcttatttctccctcaattccttatggagaaattgctgttcctccgtcaaaatcacattctctacgcgcgatcctttttgcctccttatccaaagggacctctatcatagaaaactgtctcttctctcccgattcccaagctatgcttacagcctgtgagaaaatgggagctcacgttagaagaataggagactccttacatatccaggggaatcccgatccccatcactgtcacccacgctatttccatatggggaattctggtatcgcccttcgattcctaaccgccctttctactttatcccccacccccactttgatcacaggatcccacacactcaaacgacgtcctatagcgcctcttctatcaagcttaaaacagcttggtgcgcacattcgccaaaaaacatcttcttctattccctttaccatccatggtccattatcccctggccatgttactatctctggacaagattcccaatacgcatcagcattagcaatcactgcagctttagctccatatcccctttctttttctatcgaaaatcttaaggaacgtccttggtttgatctgaccttagattggctacactctttaaacatctctttcttaagagaccaagattctttaactttccccggaggacaatcattagaaagtttttcttattctgtgcctggagactatagttctgctgcttttttagcttcctttggtctactctcttcttcttctaaaccaactattctccgtaatctttcttctcaagattctcaaggggacaagcttctcttctctttgttaaaacaacttggagcccatattcttattggaaaacatcatatcgaaatgcacccctcttctttctccggaggtgaaattgatatggatccattcatagatgcattacccatccttgctgtcctctgctgctttgcaaaaaatccatcgcgcttgtataatgcgttgggagcaaaggacaaagaaagcaatcgcattgaagccattgcccatgaattgcaaaaaatgggtggttctgtccaccctactcgtgacggtctatatatagagccctcgcggttacatggtgcggttgttgattctcataatgatcaccgtattgctatggctctcgctgtagctggagttcatgcctcgtccggacaaaccctcctctgtaacacacagtgtataaataagagttttccatatttcgtgattgcagcgcagacactacatgccaacgttcgacactaccaagcagattttcctttgcggtcttccttctgtaggtaa

SEQ ID NO: 41 - protein, 228 aa, Chlamydia trachomatis
MLNETLFVLQILVVIGFGAFFAARNLIMLAAWASLLSIIMNIFVLKQIVLFGFEVTAADVYVIGLFSCLNCAREFWGKESTRKVIFVSWCSTLSFLILTQLHLHLKPSPGDISQLHYEALFAPSLRIISASVITTMIVQFVDFKVFGWLKKHSQGRVFGLRSACSVALSQSIDTVIFSFLGLYGLVANLPDVMMFSLLSKGTALLLASPCVALAKVFYNRLNKEEAHF

SEQ ID NO: 42 - DNA, 687 bp, Chlamydia trachomatis
atgttaaacgagacattatttgtattgcaaatccttgtagttattgggttcggagctttttttgctgcgcgtaatctaattatgttagcggcatgggcctcattgctttccattatcatgaacatttttgtattaaagcaaatcgtgttattcggattcgaagtaactgcagcggatgtttacgtgatagggctgttttcttgcttgaattgtgcgagagaattctgggggaaggagtctacaagaaaagtgatttttgtttcttggtgcagcacgctttcttttctaatcctgacacaactccatctccatcttaagccttctccaggagatatcagccaactgcactatgaagctctattcgccccttctcttcggattatttcagcatcagtgatcacaacgatgattgtgcagtttgttgattttaaggtgtttggttggctgaaaaaacattcgcaaggacgggtctttggattgcgttccgcatgctccgttgcgctttctcaaagcatagacaccgtaattttttcttttctaggtttgtatggactcgttgctaacttaccagatgtcatgatgttttctttgttatccaaagggacggctcttttgttagcttctccttgtgtggctctagccaaggttttttataatcgcttgaataaagaagaagcacacttttaa

SEQ ID NO: 43 - protein, 285 aa, Chlamydia trachomatis
MSDSDKIINDCRFDFNTTIHGDLLASNLTTEGDVTVKSISAKESFSVKRNVDVNENDIIVNGFTGAAGYDLTTQGKISINLNGNRLSNVKRPEKDSQPVPANYIRTPEYYFCSLQDGARIEWKRGQKLPLIGPSRLVYQSSRIDEFIRFVSFEEDKTKNQVKINLSGTTGLQMLAKGVYIINVGVGKRWGWNNGYGGDYCLAVPLGKEYSESSTFSRGGYYASTAVGTAIHIRKESTNPDGPFSSSDTELMKTLLEVRYKGGDYVDKSALSTLYFGVLVYPEIGG

SEQ ID NO: 44 - DNA, 858 bp, Chlamydia trachomatis
atgagtgattctgacaaaattattaatgattgtcggttcgactttaatacaactattcatggagatcttttagcttcaaatctgactacggaaggggacgttacggtaaagagtatttccgcaaaagaatccttttctgtgaaaagaaatgttgatgtgaatgagaacgacatcattgttaacggttttaccggtgccgcaggatatgatctgacaactcaaggcaaaatttcaatcaatctcaacggtaatcgacttagtaatgtcaaacgcccggagaaagactcccaaccagttcctgctaactatattcgtactcctgaatactatttctgctcattgcaagatggagcaagaatcgaatggaaacgggggcagaagcttcctctaatcgggccttcgcgcttggtgtatcaatcgtctcgtattgatgagttcattcgttttgtatcgtttgaagaagataaaactaagaatcaggtgaaaataaatctctcagggactacaggcctgcaaatgcttgcgaaaggtgtgtacattatcaacgtaggagttgggaagcgatgggggtggaataatggatatggaggagattactgtttagcggtccctttaggaaaggaatacagtgagagctctacatttagtagaggaggatactatgcttctactgctgtaggaacagcaattcatatcagaaaagagagcacaaatcctgacggacctttttcttcttcagatacagaacttatgaagacacttttagaggtgcgttacaagggcggagactatgtggacaagtccgccttgtccactttatattttggagtgctcgtatacccagagataggaggataa

SEQ ID NO: 45 - protein, 173 aa, Chlamydia trachomatis
MKKFLLLSLMSLSSLPTFAANSTGTIGIVNLRRCLEESALGKKESAEFEKMKNQFSNSMGKMEEELSSIYSKLQDDDYMEGLSETAAAELRKKFEDLSAEYNTAQGQYYQILNQSNLKRMQKIMEEVKKASETVRIQEGLSVLLNEDIVLSIDSSADKTDAVIKVLDDSFQNN

SEQ ID NO: 46 - DNA, 522 bp, Chlamydia trachomatis
atgaaaaagttcttattacttagcttaatgtctttgtcatctctacctacatttgcagctaattctacaggcacaattggaatcgttaatttacgtcgctgcctagaagagtctgctcttgggaaaaaagaatctgctgaattcgaaaagatgaaaaaccaattctctaacagcatggggaagatggaggaagaactgtcttctatctattccaagctccaagacgacgattacatggaaggtctatccgagaccgcagctgccgaattaagaaaaaaattcgaagatctatctgcagaatacaacacagctcaagggcagtattaccaaatattaaaccaaagtaatctcaagcgcatgcaaaagattatggaagaagtgaaaaaagcttctgaaactgtgcgtattcaagaaggcttgtcagtccttcttaacgaagatattgtcttatctatcgatagttcggcagataaaaccgatgctgttattaaagttcttgatgattcttttcaaaataattaa

SEQ ID NO: 47 - protein, 89 aa, Chlamydia trachomatis
MSLDKGTKEEITKKFQLHEKDTGSADVQIAILTEHITELKEHLKRSPKDQNSRLALLKLVGQRRKLLEYLNSTDTERYKNLIARLNLRK

SEQ ID NO: 48 - DNA, 270 bp, Chlamydia trachomatis
ctattttctcaaattgaggcgagcaattaaatttttatatctttcagtatcagtagaatttaagtactctaggagctttcttctctgccctactaattttagcaaagctagacgagaattttgatctttaggagatcttttaaggtgctccttgagttccgttatgtgctcagtcagaatagcaatctgcacatctgccgaacctgtgtctttttcatgaagttgaaattttttagtaatttcttctttagtgcccttatccaaagacat

SEQ ID NO: 49 - protein, 274 aa, Chlamydia trachomatis
MFTDKETHRKPFPTWAHLLHSEPSKQFVFGNWKMNKTLTEAQTFLKSFISSDILSNPQIITGIIPPFTLLSACQQAVSDSPIFLGAQTTHEADSGAFTGEISAPMLKDIGVDFVLIGHSERRHIFHEQNPVLAEKAAAAIHSGMIPVLCIGETLEEQESGATQDILLNQLTTGLSKLPEQASFILAYEPVWAIGTGKVAHPDLVQETHAFCRKTIASLFSKDIAERTPILYGGSVKADNARSLSLCPDVNGLLVGGASLSSENFLSIIQQIDIP

SEQ ID NO: 50 - DNA, 825 bp, Chlamydia trachomatis
atgtttacagacaaagaaactcacagaaaaccatttccaacttgggcccaccttctccactctgagccatcaaagcaatttgttttcggtaattggaaaatgaacaaaacacttactgaagctcagacctttttaaaaagtttcatctctagtgacattctgtctaatccccaaatcattacaggaatcattcctcctttcacactgctgtcagcttgtcaacaagctgtaagcgattcccccatctttcttggagcccaaaccactcatgaagctgactcaggagcttttactggtgagatttcagccccaatgctcaaagatatcggagtcgattttgttctcatcggacattccgaaagacgtcatatctttcatgaacaaaatcctgtacttgctgaaaaagctgctgcagctatccatagtggaatgattccagttctgtgtattggagaaactctagaagaacaagaatctggagcaactcaagatattcttttaaatcaactgactacaggattatctaaactccctgagcaagcctctttcattctagcttatgaaccagtctgggctataggcaccggaaaagtagctcatcctgatctagttcaggaaacccatgctttctgtagaaaaacgattgcttctctcttttccaaagatattgcggaacgcacccccattctttacggaggatctgtgaaagccgataatgctcgctcactttccctctgccctgatgttaatggtcttttagttggaggagcctctttatcttcagagaatttcttatccattatacaacaaatcgatatcccataa

SEQ ID NO: 51 - protein, 203 aa, Chlamydia trachomatis [sequence omitted]

SEQ ID NO: 52 - DNA, 612 bp, Chlamydia trachomatis
ctatatcaatgcacgaatctgtaagagagcttggtcaacagaagcctctgttggcaagagggcatctaaaaccttgtacctatctggagctttttctgctaaagcaagaaatccttctctgacagaccggtggaaaatttgtggtttttgctcaaatttatccagatgtttctgacgagcctttcgtagtaatccttctcttgctgggatatccaataagaatgtgatgtctggcaagaacggcttatctcccacaacatgataacataagttcgtaacaaaactctcccctaagcctccagcaattccttgatatacaatagtagaatcgtgaaaacgatcgcttataaccgtcttcccagacttaagagcaggtatgatcttttcctgaatgtgttgtgcacgagctgctaaaaacaacaacaattctgcatatggagatattttttgttctggatccagaagaaggcctcgaacactgtctccaagagagcatccccctggctctctcgtagtgacaatttctctgccttcttctattaaacgcttagaaagtgcttgtataaactgagttttcccagcaccttctccgccttctactacaataaacac

SEQ ID NO: 53 - protein, 487 aa, Chlamydia trachomatis
MSLSSSSSSDSSNLKNVLSQVIASTPQGVPNADKLTDNQVKQVQQTRQNRDDLSMESDVAVAGTAGKDRAASASQIEGQELIEQQGLAAGKETASADATSLTQSASKGASSQQCIEDTSKSLELSSLSSLSSVDATHLQEIQSIVSSAMGATNELSLTNLETPGLPKPSTTPRQEVMEISLALAKAITALGESTQAALENFQSTQSQSANMNKMSLESQGLKIDKEREEFKKMQEIQQKSGTNSTMDTVNKVMIGVTVAITVISVVSALFTCGLGLIGTAAAGATAAAAGATAAATTATSVATTVATQVTMQAVVQVVKQAIIQAVKQAIVQAIKQGIKQGIKQAIKQAVKAAVKTLAKNVGKIFSAGKNAVSKSFPKLSKVINTLGSKWVTLGVGALTAVPQLVSGITSLQLSDMQKELAQIQKEVGALTAQSEMMKAFTLFWQQASKIAAKQTESPSETQQQAAKTGAQIAKALSAISGALAAAA

SEQ ID NO: 54 - DNA, 1464 bp, Chlamydia trachomatis
atgtccctttcatcttcttcgtcttccgatagtagcaaccttaagaatgtcttgtcgcaagtcatagcttcgactcctcaaggcgttcctaatgcagataaattaaccgacaatcaggttaagcaagttcaacagacgagacaaaatcgcgatgacctaagcatggaaagcgatgtcgctgttgccggaactgctggaaaagatcgcgcagcttctgcttctcaaatagaaggacaagaacttatagagcagcaaggattagctgcagggaaagaaactgcatctgccgatgcgacatccctaacccaaagcgcatctaaaggagctagctcgcaacaatgcatagaagatactagcaaatctttagagctatcttctttaagttcgttgtcatctgtagatgccacgcatctacaagaaattcaaagcatcgtatcctctgctatgggtgctactaacgagctttccttgacgaacttagaaactccaggactacccaaaccttcaacgacacctcgtcaagaagtaatggaaattagccttgcattagcaaaagcaattaccgctcttggagagtcaacgcaagcagcattggagaacttccaaagtacgcagtcgcaatctgcgaacatgaacaaaatgtctctagaatctcaaggccttaaaattgataaagagcgtgaagagttcaaaaaaatgcaagagatccagcaaaagtctggaaccaactctaccatggataccgttaacaaagtgatgattggggttaccgtggctattactgtgatctctgtagtatccgcattattcacttgcggtcttggcttgatcggaactgctgctgcaggagccacagcagccgcggctggagctacagcagcagcaacgacagcaacttctgtagctacaacagtcgctacacaagtgactatgcaagcagtcgtgcaagtggttaaacaagctattattcaagctgttaaacaggctatcgtccaagctattaaacaagggattaaacaagggatcaaacaagccattaagcaagctgttaaggcggctgtgaaaacccttgctaaaaacgtgggtaaaattttcagcgcagggaaaaatgctgttagcaaatcgttccctaaactctccaaagttatcaacactttgggaagtaaatgggtaaccttaggagtaggagctcttacagcagttcctcaactcgtatccgggattactagtctgcagctgtcagacatgcagaaagaactggcccaaattcaaaaagaagtcggagctctcacagctcaatctgaaatgatgaaagctttcacattgttctggcaacaagcaagtaaaattgcagctaaacaaacagaaagccctagtgaaacgcaacagcaggcggccaaaaccggagctcagatagcgaaagctttgtccgcaataagtggcgccttagccgccgcagcttaa

SEQ ID NO: 55 - protein, 174 aa, Chlamydia trachomatis
MLFWGIFSLCLGGLFGGYCRLRYTAKALLLSWRQLLRLALKKREVLQEIAALQTFPLLRLEEEIAFLKQGSFYSLKEFLKASDADGVTFYEMERFFTLRLKQTLASLQESLHQEAVQHLMEELLAYENAFSFEAFAFEKAAETYATLHGHPVIQFSGKLFRFPQISFPPLDEAI

SEQ ID NO: 56 - DNA, 522 bp, Chlamydia trachomatis
atgcttttttggggcatttttagtttgtgcttaggagggttattcgggggttattgtcgcttgcgctatacagcaaaggctcttttgttatcctggcgacaactccttcggcttgccttaaaaaaaagagaggttttacaagagatcgcagcgttgcaaacattccctctccttcgtttagaagaggagatagcctttttaaagcaaggctccttctattctttgaaagaatttcttaaagctagtgatgcggatggagttactttctatgagatggaacgattttttactctccgattgaaacagacattagcatcgttgcaagaaagtttgcatcaagaggctgtccagcatttaatggaagaactacttgcgtatgagaatgcgttttcttttgaggcctttgctttcgaaaaagccgcggaaacctatgcgactcttcacggtcatccggtaatccaattttctgggaaactttttcgttttccgcaaatctcctttccgcctttagatgaagcgata

SEQ ID NO: 57 - protein, 226 aa, Chlamydia trachomatis
MTLLILLRHGQSVWNQKNLFTGWVDIPLSQQGIQEAIAAGESIKHLPIDCIFTSTLVRSLITALLAMTNHSSQKVPYIVHEERPDMSRIHSQKEMEQMIPLFQSSALNERMYGELQGKNKQEVAAQFGEEQVKLWRRSYRIAPPQGESLFDTGQRTLPYFQERIFPLLQQGKNIFISAHGNSLRSLIMDLEKLSEEQVLSLELPTGQPIVYEWTGQKFTKHAPSLG

SEQ ID NO: 58 - DNA, 681 bp, Chlamydia trachomatis
ttaaccaagagaaggagcgtgtttcgtgaatttttgtcccgtccattcgtatacaataggctgtcctgttggcaactccaaagagagtacttgttcttcagataatttttctaggtccataattaaggagcgcaaagaattcccgtgagcagagataaaaatatttttcccttgctgaaggagagggaaaattctctcttgaaaataggggagggttcgttgccctgtatcgaaaagactttcgccctgaggaggggcaatgcggtagcttcggcgccacagttttacctgttcttctccgaattgagcagcgacttcttgtttattttttccttgaagttctccgtacatgcgttcattgagagcgctagattgaaaaagagggatcatctgctccatttctttttgactatgaatccggctcatgtcggggcgctcttcatgaacgatataaggaactttttgagagctgtggttagtcattgctaacagggctgttatcaaacttctaaccaaggtggaagtgaagatgcaatcaataggaagatgtttaatagattctccagcggcaatagcctcttgaattccttgttggctaagagggatgtctacccagcctgtaaacagatttttttgattccatacggattggccatggcgtagcaagataagaagcgtcat

SEQ ID NO: 59 - protein, 157 aa, Chlamydia trachomatis
MKPLKGCPVAKDVRVAIVGSCFNSPIADRLVAGAQETFFDFGGDPSSLTIVRVPGAFEIPCAIKKLLSTSGQFHAVVACGVLIQGETSHYEHIADSVAAGVSRLSLDFCLPITFSVITAPNMEAAWERAGIKGPNLGASGMKTALEMASLFSLIGKE

SEQ ID NO: 60 - DNA, 474 bp, Chlamydia trachomatis
atgaaaccgttgaaaggatgtcctgtcgctaaggatgtgcgtgtagctattgttgggtcatgtttcaattctcctatcgctgataggcttgttgctggggcgcaagaaacctttttcgatttcggaggagatccttcttctttaacaattgtccgagtccctggggcgtttgagattccttgtgcgattaagaaattactttccacctcaggacagtttcatgctgtggttgcttgcggagtgttgattcagggcgagacatcgcattatgaacatatagcagatagtgtggctgcaggtgttagtcgcctatccttagacttctgtcttcctattacattttccgtgattactgctcctaatatggaagcggcttgggagcgtgcgggtatcaaagggcccaatttaggcgcttcaggcatgaaaacagctttagaaatggcatcattattctctctgatagggaaggaataa

SEQ ID NO: 61 - protein, 166 aa, Chlamydia trachomatis
MNSGMFPFTFFLLYICLGMLTAYLANKKNRNLIGWFLAGMFFGIFAIIFLLILPPLPSSTQDNRSMDQQDSEEFLLQNTLEDSEIISIPDTMNQIAIDTEKWFYLNKDYTNVGPISIVQLTAFLKECKHSPEKGIDPQELWVWKKGMPNWEKVKNIPELSGTVKDE

SEQ ID NO: 62 - DNA, 501 bp, Chlamydia trachomatis
atgaactccggaatgttcccattcaccttttttttactgtacatctgtctgggaatgcttacggcgtacctagctaataaaaaaaatcgcaatctaataggctggtttttggcaggaatgttttttggtatttttgccattatcttcctattaattctccctcctcttccttcttctacacaagataatcgttccatggaccagcaagattccgaagaattccttttacagaatactttagaggactcagaaattatttccatcccagatacaatgaatcaaattgcgattgatacagaaaagtggttctacttaaataaagactatactaatgtcggtcctatttccatcgtacagctgaccgcattcttaaaagaatgcaaacactctcctgaaaaagggatcgatccccaagaattatgggtatggaagaaaggaatgcctaactgggaaaaggtgaagaatataccggaactttcaggaacagtaaaagacgagtaa

SEQ ID NO: 63 - protein, 321 aa, Chlamydia trachomatis
MKRLFFICALALSPLAYGAVQKDPMLMKETFRNNYGIIVSKQEWNKRGCDGSITRVFKDGTTTLEVYAQGALHGEVTRTFPHSTTLAVIETYDQGRLLSKKTFFPNALPAKEEVYHEDGSFSLTRWPDNNNSDTITDPCFVEKTYGGRVLEGHYTSFNGKYSSTILNGEGVRSTFSSDSILLTEESFNDGVMVKKTTFYSTREPETVTHYVNGYPHGVRFTYLPGGIPNTIEEWRYGHQDGLTILFKNGCKIAEVPFVRGAKNGIELRYNEQENIAEEISWQHNILHGVRKIHAAGVCKSEWYYKGKPVSQIKFERLSAAR

SEQ ID NO: 64 - DNA, 966 bp, Chlamydia trachomatis
atgaagcgtttattttttatctgcgccctcgccctttctcctctagcatatggagctgttcaaaaggatcctatgttaatgaaggagactttccgtaataactacgggatcattgtctctaagcaagaatggaacaaacgtggatgcgatggctccatcactagagtattcaaagatggaactacaaccttagaagtttatgcgcaaggtgctttacatggggaagtcacacgaacgtttcctcactctactaccctggccgttatagaaacttatgatcagggaaggcttctttctaagaagaccttcttcccaaatgctttgcctgctaaagaagaagtttaccacgaagatgggtctttctccctaacacgttggcctgacaataacaactctgacacaatcacagacccctgctttgtagaaaaaacttatgggggaagagtattggaaggtcattacacctcttttaatggaaaatactcttcaacaatccttaacggcgagggagttcgctctactttttcttcggatagtatcttgttgacagaagagtcgtttaatgatggcgtaatggtcaaaaaaacgacattttactcgactcgagaacccgaaaccgtcactcattatgtcaatgggtaccctcacggagttcggtttacctatcttcctggtgggattccaaatacgattgaagaatggcgatatggacatcaagacggccttacaatcttatttaaaaatggttgtaagattgctgaagtcccatttgtacgcggagcaaaaaatggaatcgaactccgatacaatgaacaagagaatatcgctgaagagatttcttggcagcacaacatcttgcatggagtccgtaaaatccatgcggcgggggtatgcaaatccgaatggtattacaaaggcaaacctgtctcgcaaatcaagtttgaacgactcagcgctgccagataa

SEQ ID NO: 65 - protein, 102 aa, Chlamydia trachomatis
MQNKRKVRDDFIKIVKDVKKDFPELDLKIRVNKEKVTFLNSPLELYHKSVSLILGLLQQIENSLGLFPDSPVLEKLEDNSLKLKKALIMLILSRKDMFSKAE

SEQ ID NO: 66 - DNA, 306 bp, Chlamydia trachomatis
atgcaaaataaaagaaaagtgagggacgattttattaaaattgttaaagatgtgaaaaaagatttccccgaattagacctaaaaatacgagtaaacaaggaaaaagtaactttcttaaattctcccttagaactctaccataaaagtgtctcactaattctaggactgcttcaacaaatagaaaactctttaggattattcccagactctcctgttcttgaaaaattagaggataacagtttaaagctaaaaaaggctttgattatgcttatcttgtctagaaaagacatgttttccaaggctgaa

SEQ ID NO: 67 - protein, 208 aa, Chlamydia trachomatis
MADLSAQDKLKQICDALREETLKPAEEEAGSIVHNAREQAKRIVEEAKEEAQRIIRSAEETADQTLKKGEAALVQAGKRSLENLKQAVETKIFRESLGEWLDHVATDPEVSAKLVQALVQAVDAQGISGNLSAYIGKHVSARAVNEALGKEITSKLKEKGVSVGNFSGGAQLKVEERNWVLDMSSEVLLDLLTRFLQKDFREMIFQSC

SEQ ID NO: 68 - DNA, 627 bp, Chlamydia trachomatis
atggcagatctcagcgctcaagataaattaaagcaaatatgtgatgctttgcgagaggaaactttaaaaccagctgaagaggaagctggttctattgttcataatgcaagagagcaagcaaaacgtattgttgaggaggccaaggaagaggcgcaaaggattattcgttctgcggaagagacagctgaccaaactctgaaaaaaggagaggcggctttggtacaggcaggaaagcgttctttggaaaacttgaagcaggcagtagaaacgaagatcttcagagagtctttgggtgaatggttagatcatgtggctacagatccagaagtcagcgctaagctcgtgcaagctttagtgcaggcagttgatgcacaagggatttctgggaatctttctgcctatatagggaaacacgtgtcagctcgagctgtcaatgaggctttagggaaagagataacttctaagcttaaagagaaaggggtatctgttggcaatttttctggaggtgctcagttaaaagttgaagagcgcaattgggttttagatatgagctcagaggttttgctagatttattgactagatttttacagaaagattttcgggaaatgatctttcagtcttgctaa

SEQ ID NO: 69 - protein, 255 aa, Chlamydia trachomatis
MNTLGPYHKRVRFITYLFVAFGIIVSWNLPRSAYESIQDTFVRVCSKFLPFRQGSDSLALVEETQCFLLKEKIRLLEERILSMEEAKQSPPLFSEILSSYFQSPIMGRVIFRDPAHWGSSCWINIGKRQGVKKNSPVVCGKVVVGLVDFVGEAQSRVRFITDVGIKPSVMAVRGEIQTWVVKDQLRTLARNVANLPASAFADSDKQEALHLLQALEDSLSLSEQNDFALRGIVCGRGDPIWKPEASILSGTILVL

SEQ ID NO: 70 - DNA, 768 bp, Chlamydia trachomatis
atgaataccctcggtccgtatcataaacgcgttcggttcattacgtatctttttgttgccttcgggattattgtgagttggaatcttcctcgaagtgcttacgagtctatccaggatacattcgttcgggtgtgttccaaatttcttccatttcggcaagggtctgattctctggcccttgttgaagaaactcaatgctttttattgaaagaaaaaattcgtttattggaagagcgtattctttctatggaagaggcaaaacagtctccgcctttgttttcagaaattctatcctcgtattttcaatctcccattatgggaagagttatctttcgagatccagcacactggggtagttcttgttggattaatataggaaagcgacagggcgttaaaaagaattctcctgttgtttgcggtaaggttgttgtggggttggtggattttgttggtgaagcgcagtctcgtgtacgattcatcaccgatgtgggtatcaaaccttctgttatggcggttcgtggtgaaattcaaacttgggttgtgaaagatcagctacgtacattagctaggaacgtcgctaatcttccggcatctgcttttgcagatagtgataaacaggaagctttacatctcttgcaggctctagaggattctttatctctatcagaacaaaatgattttgctcttcgtggaattgtttgtggtcgtggggatcctatttggaaaccggaggcttctatacttagcggtacgattttggttttgtag

SEQ ID NO: 71 - protein, 163 aa, Chlamydia trachomatis
MNYHNTFVKTSMFFLAKRLVQLNKNPFLLKKFSETTVLFIFERQLKMWEGYSIDENNYISDYNMEFGRPLLQKLANPVCKALLQKQLEAEQAMTLSNQVTVGDIVLMRSPIFEKSVLLETLINEIIYQESLFLFKKPENVQCPKMSFEHGAHEILLKIFLTVS

SEQ ID NO: 72 - DNA, 489 bp, Chlamydia trachomatis
atgaattatcacaacacttttgtaaaaaccagcatgtttttcttggcaaaaagactagttcagttaaataaaaatcctttcttactcaaaaagttttcagaaacaacggttctttttatattcgaacgacaacttaaaatgtgggaaggttattctatagacgagaataattatatatctgattataacatggaatttgggcgacctttattacaaaaactagcaaatccagtatgcaaagctttgttgcaaaaacagctcgaagccgagcaagcaatgacgttatccaatcaagtcactgttggagatatagtgcttatgcgttctccaattttcgaaaaatctgtattattagaaactttaatcaacgagattatttatcaagaatcgttatttttgtttaagaaaccagaaaatgttcaatgtccgaagatgagtttcgagcacggtgcacacgaaatcttgttgaagatctttttgacggtctca

SEQ ID NO: 73 - protein, 553 aa, Chlamydia trachomatis
MRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 74 - DNA, 1662 bp, Chlamydia trachomatis
atgcgaataggagatcctatgaacaaactcatcagacgagcagtgacgatcttcgcggtgactagtgtggcgagtttatttgctagcggggtgttagagacctctatggcagagtctctctctacaaacgttattagcttagctgacaccaaagcgaaagacaacacttctcataaaagcaaaaaagcaagaaaaaaccacagcaaagagactcccgtagaccgtaaagaggttgctccggttcatgagtctaaagctacaggacctaaacaggattcttgctttggcagaatgtatacagtcaaagttaatgatgatcgcaatgttgaaatcacacaagctgttcctgaatatgctacggtaggatctccctatcctattgaaattactgctacaggtaaaagggattgtgttgatgttatcattactcagcaattaccatgtgaagcagagttcgtacgcagtgatccagcgacaactcctactgctgatggtaagctagtttggaaaattgaccgcttaggacaaggcgaaaagagtaaaattactgtatgggtaaaacctcttaaagaaggttgctgctttacagctgcaacagtatgcgcttgtccagagatccgttcggttacaaaatgtggacaacctgctatctgtgttaaacaagaaggcccagagaatgcttgtttgcgttgcccagtagtttacaaaattaatatagtgaaccaaggaacagcaacagctcgtaacgttgttgttgaaaatcctgttccagatggttacgctcattcttctggacagcgtgtactgacgtttactcttggagatatgcaacctggagagcacagaacaattactgtagagttttgtccgcttaaacgtggtcgtgctaccaatatagcaacggtttcttactgtggaggacataaaaatacagcaagcgtaacaactgtgatcaacgagccttgcgtacaagtaagtattgcaggagcagattggtcttatgtttgtaagcctgtagaatatgtgatctccgtttccaatcctggagatcttgtgttgcgagatgtcgtcgttgaagacactctttctcccggagtcacagttcttgaagctgcaggagctcaaatttcttgtaataaagtagtttggactgtgaaagaactgaatcctggagagtctctacagtataaagttctagtaagagcacaaactcctggacaattcacaaataatgttgttgtgaagagctgctctgactgtggtacttgtacttcttgcgcagaagcgacaacttactggaaaggagttgctgctactcatatgtgcgtagtagatacttgtgaccctgtttgtgtaggagaaaatactgtttaccgtatttgtgtcaccaacagaggttctgcagaagatacaaatgtttctttaatgcttaaattctctaaagaactgcaacctgtatccttctctggaccaactaaaggaacgattacaggcaatacagtagtattcgattcgttacctagattaggttctaaagaaactgtagagttttctgtaacattgaaagcagtatcagctggagatgctcgtggggaagcgattctttcttccgatacattgactgttccagtttctgatacagagaatacacacatctattaa

SEQ ID NO: 75 - protein, 284 aa, Chlamydia trachomatis
MFKKFKPVTPGTRQLILPSFDELTTQGELKGSSSRRSVRPNKKLSFFKKSSGGRDNLGHISCRHRGGGVRRHYRVIDFKRNKDGIEAKVASVEYDPNRSAYIALLNYVDGEKRYILAPKGIKRGDRVISGEGSPFKTGCCMTLKSIPLGLSVHNVEMRPGSGGKLVRSAGLSAQIIAKTAGYVTLKMPSGEFRMLNEMCRATVGEVSNADHNLCVDGKAGRRRWKGIRPTVRGTAMNPVDHPHGGGEGRHNGYISQTPWGKVTKGLKTRDKRKSNKWIVKDRRK

SEQ ID NO: 76 - DNA, 855 bp, Chlamydia trachomatis
atgtttaaaaagtttaagccagtaactcccgggacgagacagttaattctgccttcttttgatgagcttactactcaaggagagttaaagggatctagttctagaagaagtgttcgtccaaataaaaagctttcttttttcaaaaagagctctggaggacgagataatttaggacatatttcctgccgccatcgtggaggaggagtaagacgtcattatagagtgatcgacttcaaacgtaataaagacggtattgaagcgaaggttgcttctgtggagtatgatccaaaccgttctgcttatattgctctattgaattatgtagatggagaaaagcgttatattctagctcctaaaggaattaagcgaggcgatcgtgtgatttctggagaaggaagtcctttcaaaactggatgctgcatgactcttaagagcatccctctgggactttctgttcataacgtggagatgagacctggctccgggggtaaattagtccgttctgcaggactttcagcccagatcatcgctaaaacagctggatacgtcactttgaagatgccttctggcgaatttcgtatgttgaatgaaatgtgccgagctactgtcggagaggtctccaatgcagatcacaatctgtgtgtagacggtaaagctgggcgtcgtcgatggaaaggaattcggccaacagttcgaggaacagctatgaaccctgttgatcacccacacggaggtggtgaagggcgtcataacggatacatttcccagaccccttggggtaaagtcacgaaaggattgaaaactcgtgataagcgtaagagtaataagtggatagttaaggatagaaggaaatag

SEQ ID NO: 77 - protein, 209 aa, Chlamydia trachomatis
MKILIASSHGYKVRETKVFLKKLGEFDIFSLVDYPSYHPPKETGETPEENAIQKGLFAAQTFRCWTIADDSMLIIPALGGLPGKLSASFAGEQANDKDHRKKLLENMRLLENTIDRSAYFECCVALISPFGKIFKAHASCEGTIAFEERGSSGFGYDPLFVKHDYKQTYAELPEAIKNQVSHRAKALVKLQPYVETVLANHLLAGKESL

SEQ ID NO: 78 - DNA, 630 bp, Chlamydia trachomatis
atgaaaattcttatagccagttctcatggatataaggtgcgcgaaaccaaggtttttctaaaaaaactaggagagtttgatatcttctcgcttgtagactacccatcctaccacccccctaaggaaactggcgaaaccccagaagaaaatgctattcagaaaggcttatttgcagctcaaacctttcgttgttggactattgctgatgattctatgcttatcattccagctttaggtggactcccaggaaaattatccgcttcttttgctggagaacaggcaaacgataaagatcatcgcaaaaaacttcttgagaacatgcgtcttttagaaaatactatcgaccgatcggcttattttgaatgctgcgtcgctttaatttctccttttggaaagatcttcaaagctcacgcctcttgcgaaggaacgattgcgtttgaggaacgcggttcctcagggtttggatatgatcctttgtttgtaaaacatgactacaagcaaacttatgccgaattaccagaggcaattaaaaaccaagtttctcacagagcaaaagcattagtcaaattacagccctatgtggaaacggttctcgcaaatcacttactcgcggggaaagagagtctctaa

SEQ ID NO: 79 - protein, 424 aa, Chlamydia trachomatis
MCVSRSLRWCLCFLLLCGWVDAGVYDKLRLTGINIIDRNGLSETICSKEKLQKYTKIDFLSPQPYQKVMRTYKNAAGESVACLTTYYPNGQIRQYLECLNNRAFGRYREWHSNGKIHIQAEVIGGIADLHPSAEAGWLFDGTTYAHDSEGRLEAVIHYEKGLLEGISLYYHANGNVWKECPYHKGVAHGDFLVFTEEGSLLKKQTFCKGQLSGCVLRYEPGSQSLLSEEEYKQGKLRSGKYYDPLTKEEIACVVNGKGKQVIYGKYAIIETRQIVHGVPHGEVLLFDEHGKSLLQAYSLINGQKEGEEVFFYPGGEGRKMLLTWSQGILQGAVKTWYPNGALESSKELVQNKKTGILMLYYPEGQVMATEEYVDDLLIKGEYFRPNDRYPYAKVEKGCGTAVFFSATGGLLKKVLYEDGKPVIH

SEQ ID NO: 80 - DNA, 1275 bp, Chlamydia trachomatis
atgtgtgtaagtagaagcttaagatggtgtttatgttttcttttgctgtgcggatgggtggacgctggggtttatgataagctccgactgacaggcattaacattatcgataggaatggtctttctgagacgatctgttctaaagaaaaattacaaaagtatacgaaaatcgattttctctctcctcagccttaccaaaaagtcatgcgtacatacaaaaacgcagcaggcgagtcggttgcttgtttaacgacgtactatccgaatggccaaatccgacaatatctcgagtgtttaaataatcgtgcttttggacgttatcgtgagtggcatagtaatggcaaaattcatatccaggcagaagttattggagggatagcagatttgcatccttccgcagaagccggatggttgttcgatggaacaacgtatgcacatgatagcgaagggcggttagaagctgttattcattatgaaaaaggcttgctggaagggatttcgctgtattaccacgcgaatgggaatgtatggaaggaatgtccttaccataaaggtgttgctcatggagactttttggtcttcaccgaagaaggaagtttgttaaagaaacaaactttttgtaaagggcagttgtctggatgtgtattacgctacgagccaggttcacagtcattgttgtcagaagaagaatataaacaagggaaactgcgcagtggtaaatattacgatcctcttactaaggaagaaatcgcgtgcgtagtgaatggcaaaggtaaacaagtaatttatgggaaatatgcgattatagagacccgacagattgtacatggcgttcctcacggggaagtcttgttatttgatgaacatggtaaatctctgttgcaagcatattctctaatcaatgggcagaaagagggagaagaagtatttttctatccaggcggagaaggtagaaaaatgttattaacatggtcccaaggtattctacaaggagctgtgaaaacttggtacccaaatggcgctttggaaagtagcaaagaacttgttcaaaataaaaagactgggattctcatgctatactatcccgaaggacaagtgatggctaccgaggaatatgtagacgatcttctcataaaaggagaatatttccggccgaacgaccgatatccatatgctaaagtggaaaaaggttgtgggacagcggtctttttcagtgctacaggaggactgttaaagaaagtcctctatgaagatgggaagcctgttattcattag

SEQ ID NO: 81 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 82 - DNA, 3105 bp, Chlamydia trachomatis
atgattaaaagaacttctctatcctttgcttgcctcagttttttttatctttcaactatatccattttgcaagctaatgaaacggatacgctacagttccggcgatttactttttcggatagagagattcagttcgtcctagatcccgcctctttaattaccgcccaaaacatcgttttatctaatttacagtcaaacggaaccggagcctgtaccatttcaggcaatacgcaaactcaaatcttttctaattccgttaacaccaccgcagattctggtggagcctttgatatggttactacctcattcacggcctctgataatgctaatctactcttctgcaacaactactgcacacataataaaggcggaggagctattcgttccggaggacctattcgattcttaaataatcaagacgtgcttttttataataacatatcggcaggggctaaatatgttggaacaggagatcacaacgaaaaaaataggggcggtgcgctttatgcaactactatcactttgacagggaatcgaactcttgcctttattaacaatatgtctggagactgcggtggagccatctctgctgacactcaaatatcaataactgataccgttaaaggaattttatttgaaaacaatcacacgctcaatcatataccgtacacgcaagctgaaaatatggcacgaggaggagcaatctgtagtagaagagacttgtgctcaatcagcaataattctggtcccatagtttttaactataaccaaggcgggaaaggtggagctattagcgctacccgatgtgttattgacaataacaaagaaagaatcatcttttcaaacaatagttccctgggatggagccaatcttcttctgcaagtaacggaggagccattcaaacgacacaaggatttactttacgaaataataaaggctctatctacttcgacagcaacactgctacacacgccgggggagccattaactgtggttacattgacatccgagataacggacccgtctattttctaaataactctgctgcctggggagcggcctttaatttatcgaaaccacgttcagcgacaaattatatccatacagggacaggcgatattgtttttaataataacgttgtctttactcttgacggtaatttattagggaaacggaaactttttcatattaataataatgagataacaccatatacattgtctctcggcgctaaaaaagatactcgtatctatttttatgatcttttccaatgggagcgtgttaaagaaaatactagcaataacccaccatctcctaccagtagaaacaccattaccgttaacccggaaacagagttttctggagctgttgtgttctcctacaatcaaatgtctagtgacatacgaactctgatgggtaaagaacacaattacattaaagaagccccaactactttaaaattcggaacgctagccatagaagatgatgcagaattagaaatcttcaatatcccgtttacccaaaatccgactagccttcttgctttaggaagcggcgctacgctgactgttggaaagcacggtaagctcaatattacaaatcttggtgttattttacccattattctcaaagaggggaagagtccgccttgtattcgcgtcaacccacaagatatgacccaaaatactggtaccggccaaactccatcaagcacaagtagtataagcactccaatgattatctttaatgggcgcctctcaattgtagacgaaaattatgaatcagtctacgacagtatggacctctccagagggaaagcagaacaactaattctatccatagaaaccactaatgatgggcaattagactccaattggcaaagttctctgaatacttctctactctctcctccacactatggctatcaaggtctatggactcctaattggataacaacaacctataccatcacgcttaataataattcttcagctccaacatctgctacctccatcgctgagcagaaaaaaactagtgaaacttttactcctagtaacacaactacagctagtatccctaatattaaagcttccgcaggatcaggctctggatcggcttccaattcaggagaagttacgattaccaaacatacccttgttgtaaactgggcaccagtcggctacatagtagatcctattcgtagaggagatctgatagccaatagcttagtacattcaggaagaaacatgaccatgggcttacgatcattactcccggataactcttggtttgctttgcaaggagctgcaacaacattatttacaaaacaacaaaaacgtttgagttatcatggctactcttctgcatcaaaggggtataccgtctcttctcaagcatcaggagctcatggtcataagtttcttctttccttctcccagtcatctgataagatgaaagaaaaagaaacaaataaccgcctttcttctcgttactatctttctgctttatgtttcgaacatcctatgtttgatcgcattgctcttatcggagcagcagcttgcaattatggaacacataacatgcggagtttctatggaactaaaaaatcttctaaagggaaatttcactctacaaccttaggagcttctcttcgctgtgaactacgcgatagtatgcctttacgatcaataatgctcaccccatttgctcaggctttattctctcgaacagaaccagcttctatccgagaaagcggtgatctagctagattatttacattagagcaagcccatactgccgttgtctctccaataggaatcaaaggagcttattcttctgatacatggccaacactctcttgggaaatggaactagcttaccaacccaccctctactggaaacgtcctctactcaacacactattaatccaaaataacggttcttgggtcaccacaaataccccattagctaaacattccttttatgggagaggttctcactccctcaaattttctcatctgaaactatttgctaactatcaagcagaagtggctacttccactgtctcacactacatcaatgcaggaggagctctggtcttttaa

SEQ ID NO: 83 - protein, 19 aa, Escherichia coli
MRYLATLLLSLAVLITAGC